(19) United States
(12) Patent Application Publication
(10) Pub. No.: US 2007/0231304 A1
(43) Pub. Date: Oct. 4, 2007

(54) PROGNOSTIC FACTORS FOR ANTI-HYPERPROLIFERATIVE DISEASE GENE THERAPY

(75) Inventors: Robert E. Sobol, Rancho Sante Fe, CA (US); Sunil Chada, Missouri City, TX (US); Louis Zumstein, Del Mar, CA (US); Esteban Cvitkovic, Le Kremlin-Bicetre Cedex (FR); Kerstin Menander, Bellaire, TX (US)

Correspondence Address: FULBRIGHT & JAWORSKI L.L.P., 600 CONGRESS AVE., SUITE 2400, AUSTIN, TX, 78701, US

(73) Assignee: INTROGEN THERAPEUTICS, INC.

(21) Appl. No.: 11/668,981

(22) Filed: Jan. 30, 2007

Related U.S. Application Data
(60) Provisional application No. 60/799,471, filed on May 10, 2006.
(60) Provisional application No. 60/763,680, filed on Jan. 30, 2006.

(51) Int. Cl. A61K 48/00; A61K 31/7105; A61P 43/00; C12Q 1/68; A61K 31/711

(52) U.S. Cl. 424/93.2; 435/6; 514/44

(57) ABSTRACT

The present invention relates to the identification of various prognostic factors that predict response in patients with hyperproliferative disease such as cancer to gene therapy, and their use in methods of treating such patients with an anti-hyperproliferative disease gene therapy. Also described are methods of treatment for Li Fraumeni syndrome, and for assessing anti-cancer gene therapy using PET scans.

This application claims benefit of priority to U.S. Provisional Application Ser. No. 60/799,471, filed May 10, 2006, and U.S. Provisional Application Ser. No. 60/763,680, filed Jan. 30, 2006, the entire contents of both applications being hereby incorporated by reference.

### BACKGROUND OF THE INVENTION

I. Field of the Invention

The present invention relates generally to the fields of oncology and gene therapy. More particularly, it concerns the assessment of various patient factors to predict the efficacy of an anti-hyperproliferative disease gene therapy.

II. Description of Related Art

Cancer is a leading cause of death in most countries, and the result of billions of dollars in healthcare expense around the world. It is now well established that a variety of cancers are caused, at least in part, by genetic abnormalities that result in either the overexpression of cancer causing genes, called “oncogenes,” or from loss of function mutations in protective genes, often called “tumor suppressor” genes. An example of the latter category is p53 —a 53 kD nuclear phosphoprotein that controls cell proliferation. Mutations to the p53 gene and allele loss on chromosome 17p, where this gene is located, are among the most frequent alterations identified in human malignancies. The p53 protein is highly conserved through evolution and is expressed, albeit at low levels, in most normal tissues. Wild-type p53 has been shown to be involved in control of the cell cycle (Mercer, 1992), transcriptional regulation (Fields and Jang, 1990; Mietz et al., 1992), DNA replication (Wilcock and Lane, 1991; Bargonetti et al., 1991), and induction of apoptosis (Yonish-Rouach et al., 1991; Shaw et al., 1992).

Various mutant p53 alleles are known in which a single base substitution results in the synthesis of proteins that have quite different growth regulatory properties and, ultimately, lead to malignancies (Hollstein et al., 1991). In fact, the p53 gene has been found to be the most frequently mutated gene in common human cancers (Hollstein et al., 1991; Weinberg, 1991), and mutation of p53 is particularly associated with those cancers linked to cigarette smoke (Hollstein et al., 1991; Zakut-Houri et al., 1985). The overexpression of p53 in breast tumors has also been documented (Casey et al., 1991). Interestingly, however, the beneficial effects of p53 are not limited to cancers that contain mutated p53 molecules. In a series of papers, Clayman et al. (1995) demonstrated that growth of cancer cells expressing wild-type p53 molecules was nonetheless inhibited by expression of p53 from a viral vector.

As a result of these findings, considerable effort has been placed into p53 gene therapy. Retroviral delivery of p53 to humans was reported some time ago (Roth et al., 1996). There, a retroviral vector containing the wild-type p53 gene under control of a beta-actin promoter was used to mediate transfer of wild-type p53 into 9 human patients with non-small cell lung cancers by direct injection. No clinically significant vector-related toxic effects were noted up to five months after treatment. In situ hybridization and DNA polymerase chain reaction showed vector-p53 sequences in post-treatment biopsies. Apoptosis (programmed cell death) was more frequent in post-treatment biopsies than in pretreatment biopsies. Tumor regression was noted in three patients, and tumor growth stabilized in three other patients. Similar studies have been conducted using adenovirus to deliver p53 to human patients with squamous cell carcinoma of the head and neck (SCCHN) (Clayman et al., 1998). Surgical and gene transfer-related morbidities were minimal, and the overall results provided preliminary support for the use of Ad-p53 gene transfer as a surgical adjuvant in patients with advanced SCCHN.

Several clinical prognostic factors influencing response to therapy and survival have been identified in patients with recurrent SCCHN (Argiris et al., 2004; Pivot et al., 2001; Recondo et al., 1991). Molecular biomarkers have more recently been used to predict prognosis. Specifically, dysfunction of p53 tumor suppressor pathways has been shown to correlate with poor prognosis in a variety of malignancies including SCCHN (Recondo et al., 1991; Gallo et al., 1995; Mulder et al., 1995; Sarkis et al., 1995; Sauter et al., 1995; Stenmark-Askmalm et al., 1995; Matsumura et al., 1996; McKaig et al., 1998; Nemunaitis et al., 1991). Advances in the understanding of the critical role of abnormal p53 function in tumor proliferation and treatment resistance provided the rationale for developing p53 gene therapies for SCCHN and other cancers (Hartwell and Kastan, 1994; Kastan et al., 1995; Edelman and Nemunaitis, 2003; Ahomadegbe et al., 1995; Ganly et al., 2000; Zhang et al., 1995; Clayman et al., 1995; Clayman et al., 1998; Clayman et al., 1999; Swisher et al., 1999; Nemunaitis et al., 2000; Peng, 2005). Thus, despite gene therapy successes, it is presently unclear why some patients respond to p53 and other gene therapies while others do not. There remains a need to identify specific patient subsets that will most benefit from this treatment.

### SUMMARY OF THE INVENTION

Thus, in accordance with the present invention, there is provided a method of providing a clinical benefit to a subject suffering from a tumor comprising: (a) assessing a gene therapy treatment outcome indicator in said subject, wherein the presence of the gene therapy treatment outcome indicator correlates with clinical benefit following gene therapy; (b) making a treatment decision based on step (a); and (c) treating the subject with said gene therapy if the subject exhibits the gene therapy outcome indicator. Tumor supporessor gene therapy may be a p53 therapy (e.g., Advexin).

The present inventors have determined that disruption of the p53 pathway in a patient predicts response to a gene therapy (e.g., a p53 gene therapy or Advexin), and further have determined that increased levels of p53 or detectable p53 may be used as a prognostic indicator to predict response to gene therapy (e.g., a p53 gene therapy or Advexin). While not being limited to any theory, the inventors believe that increased levels of p53 or detectable p53 indicates an abnormality in the p53 pathway. Regardless, increased levels of p53 or detectable p53 may be used to predict response to a gene therapy.

The gene therapy treatment outcome indicator may be detectable p53 protein expression in a tumor cell from said subject, wherein detectable p53 correlates with clinical benefit following gene therapy. Evaluation of increased levels of p53 may be performed using a variety of techniques, including measuring levels of p53 protein in a cell (e.g., detectable using an immunoassay such as immunohistochemistry (IHC)). Alternatively, p53 transcripts may be measured in a cell to evaluate overexpression of or increased levels of p53 using, for example, PCR. However, it is anticipated that virtually any test for analysis of p53 may be calibrated, by comparison to p53 detection in a statistically sufficient number of non-cancerous cells, for use with the present invention. Tissues (e.g., a cancerous tumor) containing greater than about 20% cells with increased p53 levels or detectable p53 (e.g., using IHC) may indicate the increased probability of response to a gene therapy (e.g., p53 or other tumor suppressors).

In another embodiment, a defect or abnormality in the p53 pathway is detected by examining a gene or gene product upstream or downstream of p53. For example, the gene therapy treatment outcome indicator may include assessing p14ARF and/or hdm-2 expression in a tumor cell from said subject, as these gene products regulate p53. A normal or higher expression of p14ARF and/or normal or lower expression of hdm-2, as compared to a control tissue, correlate with clinical benefit following gene therapy. Other genes that are regulated by p53 could similarly be examined to provide information on the integrity of p53 signaling.

In certain embodiments, the gene therapy treatment outcome indicator also may be one or more of the following factors for the subject: (i) interval from end of first treatment after diagnosis to relapse (PFI), (ii) tumor diameter, (iii) tumor-associated pain, (iv) tumor necrosis of target lesions, (v) localization of the primary tumor, (vi) prior chemotherapy or radiotherapy, (vii) Karnofsky performance scale (KPS), (viii) weight loss, (ix) low serum albumin level; and/or (x) assessing target lesions in a prior irradiated field, wherein a PFI of greater than about 12 months, a tumor diameter of less than about 50 mm, minimal or absence of pain, absence of tumor necrosis of target lesions, absence of non-localized disease, prior exposure to chemotherapy or radiotherapy, a KPS of greater than about 90%, minimal or no prior weight loss, normal or near normal serum albumin, and the presence of target lesions in a prior irradiated field, predict clinical benefit following gene therapy.

The tumor may be a benign tumor growth (e.g., benign prostatic hyperplasia, oral leukoplakia; a colon polyp, an esophageal pre-cancerous growth, or a benign lesion.) The tumor may be cancer, such as oral cancer, oropharyngeal cancer, nasopharyngeal cancer, respiratory cancer, a urogenital cancer, a gastrointestinal cancer, a central or peripheral nervous system tissue cancer, an endocrine or neuroendocrine cancer, a hematopoietic cancer, a glioma, a sarcoma, a carcinoma, a lymphoma, a melanoma, a fibroma, a meningioma, brain cancer, oropharyngeal cancer, nasopharyngeal cancer, renal cancer, biliary cancer, prostatic cancer, pheochromocytoma, pancreatic islet cell cancer, a Li-Fraumeni tumor, thyroid cancer, parathyroid cancer, pituitary tumors, adrenal gland tumors, osteogenic sarcoma tumors, multiple neuroendrcine type I and type II tumors, breast cancer, lung cancer, head & neck cancer, prostate cancer, esophageal cancer, tracheal cancer, skin cancer brain cancer, liver cancer, bladder cancer, stomach cancer, pancreatic cancer, ovarian cancer, uterine cancer, cervical cancer, testicular cancer, colon cancer, rectal cancer or skin cancer. Clinical benefit may comprise reduction in tumor size or burden, blocking of tumor growth, reduction in tumor-associated pain, long-term non-progression, induction of remission, reduction of metastasis, or increased patient survival.

The gene therapy may be tumor suppressor gene therapy, a cell death protein gene therapy, a cell cycle regulator gene therapy, a cytokine gene therapy, a toxin gene therapy, an immunogene therapy, a suicide gene therapy, a prodrug gene therapy, an anti-cellular proliferation gene therapy, an enzyme gene therapy, or an anti-angiogenic factor gene therapy. The tumor suppressor therapy may be APC, CYLD, HIN-1, KRAS2b, p16, p19, p21, p27, p27mt, p53, p57, p73, PTEN, FHIT, Rb, Uteroglobin, Skp2, BRCA-1, BRCA-2, CHK2, CDKN2A, DCC, DPC4, MADR2/JV18, MEN1, MEN2, MTS1, NF1, NF2, VHL, WRN, WT1, CFTR, C-CAM, CTS-1, zacl, ras, MMAC1, FCC, MCC, FUS1, Gene 26 (CACNA2D2), PL6, Beta* (BLU), Luca-1 (HYAL1), Luca-2 (HYAL2), 123F2 (RASSF1), 101F6, or Gene 21 (NPRL2). The pro-apoptotic protein therapy may be mda7, CD95, caspase-3, Bax, Bag-1, CRADD, TSSC3, bax, hid, Bak, MKP-7, PARP, bad, bcl-2, MST1, bbc3, Sax, BIK, or BID. The cell cycle regulator therapy may be an antisense oncogene, an oncogene siRNA, an oncogene single-chain antibody, or an oncogene ribozyme. The cytokine therapy may be GM-CSF, G-CSF, IL-1α, IL-1β, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32 IFN-α, IFN-β, IFN-γ, MIP-1α, MIP-1β, TGF-β, TNF-α, TNF-β, or PDGF. The anti-angiogenic therapy may be angiostain, endostain, avastin or an antisense, siRNA, single-chain antibody, or a ribozyme against a pro-angiogenic factor.

The cancer cell may have a normal p53 gene and/or protein structure or an abnormal p53 gene and/or protein structure. For example, the p53 gene may produce a p53 protein which is identical to a wild-type p53 protein. In other embodiments, a mutation may exist in the p53 protein (e.g., a truncation, deletion, substitution, trans-dominant mutation, etc.). The p53 gene may be a wild-type p53 gene (i.e., the proper promoter, introns, exons, and orientation is present) or the p53 gene may have a mutation (e.g., a missense, deletion, substitution, rearrangement, etc.).

In certain embodiments, the gene therapy may be delivered by a non-viral vector. The non-viral vector may be entrapped in a lipid vehicle (e.g., a liposome). The vehicle may be a nanoparticle. The gene therapy may be delivered by a viral vector (e.g., retroviral vector, an adenoviral vector, an adeno-associated viral vector, a pox viral vector, a polyoma viral vector, a lentiviral vector, or a herpesviral vector).

In certain embodiments, the subject exhibits a higher expression of p53; for example, the subject may exhibit a higher expression in a cancerous cellrelative to a healthy cell. The subject may further exhibit 2 or more, 4 or more, 6 or more, 8 or all of the factors (i)-(x) above that correlate with clinical benefit. In certain embodiments, an additional factor is (i) or (ii) from above. In certain embodiments, p53 expression is assessed.

The gene therapy may be a loco-regional gene therapy. The loco-regional gene therapy may comprise a localized gene therapy. The localized gene therapy may comprise direct injection of the tumor, injection of tumor vasculature, regional gene therapy, or administration into a tumor-associated lymph vessel or duct. The administration may comprise intraperitoneal, intrapleural, intravesicular, or intrathecal administration. The regional gene therapy may comprise administration into the vasculature system of a limb associated with the tumor.

The assessing may comprise immunohistochemistry of a tumor sample. The assessing may comprise an ELISA, an immunoassay, a radioimmunoassay (RIA), an immunoradiometric assay, a fluoroimmunoassay, an immunoassay, a chemiluminescent assay, a bioluminescent assay, a gel electrophoresis, a Western blot analysis or an in situ hybridization assay of a tumor sample. The assessing may comprise antibody detection of p53 in a tumor cell lysate. The assessing may comprise amplification of a p53 transcript. The assessing may comprise antibody detection of p14ARF and/or hdm-2 in a tumor cell lysate. The assessing may comprise amplification (e.g., RT-PCR, in situ hybridization, Northern blotting or nuclease protection) of a p14ARF and/or an hdm-2 transcript.

In yet another embodiment, there is provided a method of treating a subject with Li Fraumeni Syndrome comprising administering to said subject an expression construct encoding p53 under the control of a promoter active in a cancer cell of said subject. The method may further comprise assessing a p53 gene in a cancer cell from said subject for mutations. The method may further comprising assessing p53 expression (e.g., using immunohistochemistry or real-time PCR) in a cancer cell from said subject for mutations. The expression construct may be a non-viral expression construct, such as a liposome or nanoparticle. The expression construct may be a viral expression construct, such as an adenoviral, a retroviral, a herpesviral, a vaccinia viral or an adeno-associated viral construct. The promoter may be a CMV IE promoter, an RSV LTR promoter, or a β actin promoter, or a telomerase promoter.

The method may further comprise administering to said subject a second anti-cancer therapy or a third anti-cancer therapy. The second and third anti-cancer therapies may be one or two or more of chemotherapy, radiotherapy, hormonal therapy, cytokine therapy, immunotherapy, and a non-p53 gene therapy or surgery. The second anti-cancer therapy may be administered to said subject prior to said p53 expression construct, administered to said subject after said p53 expression construct, or administered to said subject concurrent with p53 expression construct. The method may further comprise assessing efficacy of treatment with said p53 expression construct, such as by performing a PET scan on said subject. The p53 expression construct may be administered to said subject by intratumoral injection. Alternatively, the Li Fraumeni patient may be treated with a p53 vaccine composition, such as a dendritic cell vaccine.

In still yet another embodiment, there is provided a method of assessing the efficacy of an anti-cancer gene therapy comprising subjecting a patient that has received an anti-cancer gene therapy to a PET scan. The method may further comprise administering to said patient an anti-cancer therapy prior to said PET scan, may further comprise administering to said patient an anti-cancer therapy after said PET scan; may further comprise subjecting said patient a pretreatment PET scan; and/or may further comprise making a treatment decision for said patient based on the assessing.

“p53” as used herein, refers to a wild-type or mutant (e.g., trans-dominant, missense, etc.) p53 protein. “Detectable p53”, as used herein, refers to p53 protein that is present in a cell at a concentration sufficient for detection via immunohistochemistry or other antibody based assays (Western blot, FIA, a radioimmunoassay (RIA), RIP, ELISA, immunoassay, immunoradiometric assay, a fluoroimmunoassay, an immunoassay, a chemiluminescent assay, a bioluminescent assay, a gel electrophoresis assay). Preferably immunohistochemistry is used to detect the p53 protein. As such, detetable p53 is a way of measuring p53 overexpression relative to “normal” p53 levels, i.e., p53 levels observed in normal (non-cancerous) cells.

A “gene therapy treatment outcome indicator” refers to an indicator (e.g., the expression level of a protein such as p53, interval from diagnosis to first relapse (PFI), tumor diameter, p14ARF and/or hdm-2 expression, etc.) which may be used to predict a beneficial clinical response to the gene therapy (e.g., increased duration of survival, etc.) to a gene therapy (e.g., Advexin). Gene therapy treatment outcome indicators must be clinically, experimentally, or physically analyzed. Gene therapy treatment outcome indicators do not refer to a purely mental process.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more” or “at least one.” The term “about” means, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will be apparent to those skilled in the art from this detailed description.

### BRIEF DESCRIPTION OF THE DRAWINGS

For a more complete understanding of the present invention, reference is now made to the following description taken in conjunction with the accompanying drawing:

FIG. 1—Pet Scan of Pelvic Tumor from Li Fraumeni Patient. Left panel is the pretreatment Pet Scan of an pelvic tumor (indicated by arrow). The right panel is a Pet Scan of the same patient 59 days later, and the arrow indicates the prior location of the tumor.

FIG. 2—Pathways of p53. To assess the value of p53 pathway abnormalities in predicting Advexin® efficacy, immunohistochemical analyses of p53 protein expression and several other proteins in the p53 pathway were performed. The proteins selected for analysis are linked to regulation of p53 regulation and function, (for example, p14ARF, HDM2, bc1-2 survivin and phosphor-ser 15-p53).

FIG. 3.—Abnormal p53 Molecular Biomarker Identifies Patients with Statistically Significant Increased Survival Following Advexin Therapy in Recurrent Head and Neck Cancer. Advexin treatment of recurrent SCCHN patients with p53 abnormalities had statistically significant increased median survival compared to those whose pre-treatment tumors did not over-express p53 protein (median survival 11.6 vs. 3.5 months p<0.0007; Log Rank Test).

### DESCRIPTION OF THE ILLUSTRATIVE EMBODIMENTS

I. The Present Invention

As discussed herein, gene therapy at the clinical level has been under study for a over a decade, including a number of cancer therapy trials. Overall, the success of this approach has been promising with increased benefits over those seen with traditional therapeutic approaches. However, as with most anti-cancer treatments, there still remains a substantial need to improve the identification of patient populations that may benefit most from the efficacy of gene therapy or other medicaments.

Here, the inventors provide an analysis of a long term follow-up of a series clinical trials using p53 gene delivery. The results are applicable to other tumor suppressor gene therapies. This analysis, looking at recurrent SCCHN patients treated intralesionally with adenoviral vectors delivering the p53 gene, revealed a number of prognostic factors that identify subset populations of patients that will achieve the most benefit from intralesional gene therapy. To the inventors' knowledge, this is the first study of general prognostic factors impacting the success of a tumor suppressor gene therapy in human subjects. The present invention is exemplified in part through studies involving the use of p53 gene therapy to treat Li Fraumeni Syndrome, a cancerous condition linked to mutations in p53. Finally, the present invention provides for the use of PET scans to evaluate tumor gene therapy.

A. Assessment and Prognostic Factors

Various patient parameters, including patient/disease history/characteristics as well as molecular characteristics (e.g., overexpression of p53 in a cancerous tumor), may be used as prognostic factors to predict the response or degree of benefit to a patient from a cancer gene therapy (e.g., adenoviral p53 or other tumor suppressor therapy). These patient parameters include, in addition to overexpression of p53 in a cell or tissue as compared to non-cancerous cells, (i) interval from diagnosis to first relapse (PFI); (ii) tumor diameter; (iii) tumor-associated pain; (iv) tumor necrosis of target lesions; (v) localization of the primary tumor; (vi) prior chemotherapy or radiotherapy; (vii) Kamofsky performance scale (KPS); (viii) weight loss; (ix) serum albumin; (x) target lesions in prior radiation field; and (xi) molecular markers. Many of these factors will be assessed by merely taking a patient history, whereas others will require a physical examination, laboratory tests of urine or blood, radiographic scans and perhaps biopsy and/or pathohistology and/or molecular biology assays.

Therapy involving the use of tumor suppressor genes such as p53 can be affected by the molecular environment of the treated neoplasm. Molecular profiles of genes that positively and negatively regulate the activity of the therapeutic gene or gene protein product can predict the response of the treated subject. The degree of expression of positive and negative regulators can predict response and absence of response respectively. In addition, the degree of receptor expression by the neoplasm for the vectors utilized for gene delivery can also predict response. For example, when an adenoviral vector will be utilized for therapy, levels of tumor expression of Coxsackie-adenovirus receptors can identify high, intermediate and low responders to treatment in vitro (Tango et al., 2004). In general, for positive gene regulators and vector receptors, high or normal intermediate levels of expression identify responders while low levels are associated with poor responders. For negative therapeutic gene regulators, low or normal intermediate levels identify responders while high levels predict for a poor response to treatment.

Assessments of expression of molecular markers (e.g., increased p53 protein levels) may be direct, as in the use of quantitative immunohistochemistry (IHC) or other antibody based assays (Western blot, FIA, a radioimmunoassay (RIA), RIP, ELISA, immunoassay, immunoradiometric assay, a fluoroimmunoassay, an immunoassay, a chemiluminescent assay, a bioluminescent assay, a gel electrophoresis), or indirectly by quantitating the transcripts for these genes (in situ hybridization, nuclease protection, Northern blot or PCR, including RT-PCR).

### 1. Elevated Levels of p53 as a Prognostic Indicator of Gene Therapy Response

Elevated levels of p53 are known to signify abnormalities of the p53 tumor suppressor pathway and are associated with a poor prognosis in SCCHN cancers (Geisler et al., 2002); however, the present invention demonstrates that this subset of patients with increased p53 protein levels responds unusually well to p53 therapy. In a preferred embodiment, immunohistochemical detection of elevated levels of p53 compared to normal tissues provides an integrated measurement of several aberrant expression and/or degradation defects reflecting abnormalities in p53 pathway function. Indeed, it is contemplated that such a correlation will be evident as well in the case of gene therapy or other medicaments involving other tumor suppressor genes, particularly those that function or operate through modulation of the p53 tumor suppressor pathway. It will be known to those skilled in the art which target proteins in a clinically relevant pathway may have levels different from normal tissues indicating a defect in the pathway.

While not intending to be bound by any particular theory, the inventors propose that when a tumor cell exhibits elevated levels of p53 protein (whether mutant or normal) at a level higher than is typically seen in normal somatic cells, such an elevated level, is indicative of a dysfunction in the p53 tumor suppressor pathway, the principal pathway that regulates the cells apoptotic response to genetic mutation. It is postulated by the inventors that when there is a defect at some juncture in the pathway, that such a defect reveals itself in the elevated p53 levels. For example, it is known that when there is a defect in the p53 protein itself (i.e., resulting in a “mutant” p53), and such a defect results in a dysfunctional p53 protein, the cell overexpresses the dysfuctional protein relative to that seen in normal cells in a vain attempt to achieve a “normal” level of p53 protein function. In addition, in some instances, the mutated p53 protein may be less amenable to degradation or clearance from the cell than wild-type p53, contributing to the apparent increased p53 content of the cell.

However, the present inventors propose that even when defects occur elsewhere in the pathway (for example, in genes or genetic elements upstream or downstream of p53 protein in the pathway) or occur in, for example, non-coding regions or control elements of the p53 gene itself, that such defect(s) also can result in a disruption in the pathway, and thus lead to p53 protein elevation, again presumably due to the cell's attempt to compensate for loss or reduction of proper p53 pathway activity. Indeed, while virtually all normal somatic cells express p53 protein at near undetectable levels (e.g., detectable only by extremely sensitive techniques, such as RT-PCR), it has been found that a definable subset of tumors have elevated p53 protein, even though such protein is “normal” in terms of its primary amino acid sequence. More surprisingly, the clinical studies reviewed by the present inventors demonstrate that such “wild type” p53 protein appearing at elevated levels correlates very favorably to clinical response to tumor suppressor therapy.

The most common and convenient way of detecting such “elevated levels” of a tumor suppressor such as p53 is to select a technique that is sensitive enough to reflect or detect the protein levels commonly seen in cancer cells, yet not sufficiently sensitive to detect those levels common to normal somatic cells. Immunohistochemistry (“IHC”) techniques include a family of exemplary detection technologies applicable that can be employed to detect the “elevated level” of p53, and thus are particularly applicable to the present invention (see, e.g., Ladner et al., 2000). Conveniently, IHC techniques are not generally sensitive enough to detect the small amounts of p53 protein produced, e.g., in normal somatic cells, and for that reason are now typically employed to detect elevated levels of p53 protein. A particular advantage for practice in connection with the present invention is that IHC detection of p53 protein will not generally discriminate between wild-type and mutant or aberrant p53 protein (since the underlying antibody can be selected , preferably in the case of the present invention, to detect most p53 proteins whether mutant or normal).

Nevertheless, the present invention is of course in no way limited to the use of IHC techniques to identify and select patients having tumors with elevated levels of p53 protein, or other measurable defects in the p53 pathway, in that the invention contemplates the use of any technique that will discriminate between cells exhibiting normal and abnormal expression of p53. Examples would include detection techniques that have been appropriately calibrated to distinguish between normal and abnormal levels of p53 mRNA expression and/or p53 protein translation levels, or to detect particular mutations or defects associated with other defects in the p53 pathway that result in p53 protein elevation. Such methods will include, in addition to immunological detection of p53 proteins, nucleic acid hybridization techniques such as gene arrays and chips, that are used to detect differences in mRNA levels, and thus may be employed to discriminate p53 mRNA levels. Exemplary normal and tumor cells (in the form of cell lines) that are known to typically have normal and elevated levels of p53 protein include cells such as WI-38, CCD 16 and MRC-9, and cell lines such as SCC61, SCC173 and SCC179, obtainable from common providers such as the ATCC and others.

The present invention, with respect to detecting a defect in the p53 pathway, is in no way limited to detecting p53 protein or associated mRNA expression. Detecting such defects would include detecting defects in mRNA or protein expression for any gene in the relevant pathway, such as a mutation or dysfunction in regulation of p53 turnover, such as proteosomal regulation, ubiquitination, or in the gene expression, regulation or product turnover of HDM2, HDM4, Cop1, Pirh2, NQo1, etc., for example, that result in or correspond to an elevated level of p53 protein in cancer cells.

An important aspect of the present invention is that it is particularly directed to the application of pharmaceutical tumor suppressor therapies, such as p53, to patients that have been found to have a negative or unfavorable prognosis, as it has been reported that tumors having p53 that is measurable via immunohistochemistry correlate with decreased patient survival (Zhao et al., 2005 and Geisler et al, Clinical Cancer Research 8:3445 2002). Yet, exemplary clinical results, as set forth below, indicate that p53 therapy, and in particular, adenoviral p53 therapy, works best in the subgroup of cancer patients with elevated p53 levels, as exemplified by clinical studies carried out in head and neck SCCHN patients. In addition, patients who do not respond to other therapies and/or who have a very poor prognosis may particularly benefit from p53 or other tumor suppressor therapy based on the fact that this patient group responds to p53. Thus, it is contemplated that the present invention can provide clinical benefit to a population of patients who are considered “low responders” and “at-risk” with respect to conventional therapies.

The examples provided below demonstrate that elevated p53 level is a statistically significant marker for p53 response to such therapy. The fact that statistical significance was observed with such a small number of cases is remarkable and indicates the importance and reliability of this marker.

In certain aspects, cells that have elevated levels of p53 will thus be those cells having p53 missense mutations, null mutations, trans-dominant mutations and gain of function mutations (e.g., de Vries et al., 2002) that lead to overexpression or decreased degradation. The inventors contemplate that, particularly in patients with a p53 gain of function mutation or a transdominant mutation, a given p53 overexpressing cell, when transduced with even one particle of adenoviral p53, will produce enough wild-type p53, made by the adenovirus, to swamp out any effects of an endogenous p53 gain of function or trans-dominant allele. Furthermore, it is contemplated that such an approach will be improved by selecting a heterologous promoter, such as CMV, that is stronger that the native p53 promoter and hence capable of producing “high” levels of p53 expression, i.e., in excess of that seen in normal cells, and optionally in excess of that observed in cancer cells that “overexpress” p53.

### 2. Molecular Markers that Affect the p53 Pathway

The present invention also contemplates using molecular markers that affect the p53 pathway as prognostic indicators for response to a gene therapy (e.g., adenoviral p53). p14ARF and hdm-2 are positive and negative regulators respectively of p53 activity. A correlation between p14ARF (positive) and hdm-2 (normal or low) has been shown (Sano et al., 2000). In addition, a correlation between p14ARF (positive) and PFI has been demonstrated (Kwong et al., 2005). p14 is a positive regulator of p53 and wild-type p53 can negatively regulate p14. However, to date there have been no comparisons of clinical results using gene therapy with the existence of specific molecular and clinical markers.

As demonstrated in the examples below, the present inventors have established a correlation between PFI and clinical efficacy of gene therapy. With this information, and the previously reported association of PFI and p14ARF, the present inventors anticipate that it may be possible to provide a direct positive correlation between a molecular marker—p14ARF—and benefit from cancer gene therapy. The inventors anticipate that, given the inverse correlation between p14ARF and hdm-2, a further direct correlation (negative) between hdm-2 and gene therapy clinical benefit may exist. Thus, by looking directly at the expression of one or both of these molecules in the neoplasm of a subject, it may be possible to make a prediction about the response of that subject to p53 gene therapy.

Similarly, other regulators of p53 activity are known, and they may be similarly employed to define patients who are more likely to be potential responders or non-responders. Further examples of negative regulators or agents with antagonist p53 activity include hdm4 (Lozano and Zambetti, 2005), Cop1 (Duan et al. 2004), pirh2, (Doman et al., 2004) and inhibitors of apoptosis like BCL2 (Gallo et al., 1999). Survivin is negatively regulated by p53 (Jung et al., 2005), and the inventors anticipate that survivin may be used as molecular marker for response to a gene therapy (e.g., a p53 gene therapy, Advexin).

The inventors also anticipate that expression of FHIT (Lee et al., 2001) may be a marker predicting a positive response to gene therapy (e.g., p53 therapy). The function of the FHIT tumor suppressor inhibits HDM2 which correlates with an increased PFI>12 months in SCCHN patients (Nishizaki et al., 2004). Additionally, p14ARF, HDM2, BCL2 and/or CAR may also be used to predict response to a gene therapy, wherein increased expression of (p14ARF and/or CAR) and/or decreased expression of (HDM2 and/or BCL2) may indicate an improved response to a gene therapy (e.g., p53 therapy).

In certain embodiments, certain polymorphisms (e.g., in mdm2) may be used to predict response to gene therapy. The mdm2 polymorphism (SNP309) may serve as an important prognostic marker for p53 therapeutic activity. It has been shown that a single nucleotide polymorphism in the MDM2 promoter attenuates the p53 tumor suppressor pathway and accelerates tumor formation in humans (Bond et al., 2004). This polymorphism in mdm2 can serve as a rate limiting factor in carcinogenesis and can increase levels of the negative regulator mdm2, thereby decreasing p53 pathway function. In patients with detectable p53 in cancerous cells, p53 therapy yields improved clinical results; therefore using this polymorphism to identify those patients with the appropriate SNP309 profile will identify p53 therapy responders.

### 3. Karnofsky Performance Scale

The Karnofsky performance scale (KPS) allows patients to be classified as to their functional impairment. This can be used to compare effectiveness of different therapies and to assess the prognosis in individual patients. The lower the Karnofsky score, the worse the survival for most serious illnesses:

100 Normal, no complaints, no evidence of disease

90 Able to carry on normal activity; minor symptoms of disease

80 Normal activity with effort; some symptoms of disease

70 Cares for self; unable to carry on normal activity or active work

60 Requires occasional assistance but is able to care for needs

50 Requires considerable assistance and frequent medical care

40 Disabled; requires special care and assistance

30 Severely disabled; hospitalization is indicated, death not imminent

20 Very sick, hospitalization necessary; active treatment necessary

10 Moribund, fatal processes progressing rapidly

0 Dead

For each of the above-listed factors, the following are considered to be positive indicators for gene therapy: (i) PFI of 12 months or greater; (ii) tumor diameter of less than about 25 mm or 50 mm; (iii) absence of tumor-associated pain; (iv) absence of tumor necrosis or target lesions; (v) localization of the primary tumor; (vi) existence of prior exposure to chemotherapy or radiotherapy; (vii) KPS of 90-100; (viii) no appreciable weight loss; (ix) normal serum albumin (about 35 to about 50 g/L); (x) presence of target lesions in a prior radiation field; and (xi) presence of positive-correlating molecular markers and absence of negative-correlating molecular markers (e.g., p14ARF expression (higher)/hdm-2 expression (lower)).

B. Therapeutic Intervention

1. Therapies

In accordance with the present invention, applicants also provide methods for treating cancer in a subset of patients identified according to the methods described above. More particularly, the invention relates to treating hyperproliferative diseases. A hyperproliferative disease is a disease associated with the abnormal growth or multiplication of cells. The hyperproliferative disease may be a disease that manifests as lesions in a subject.

The hyperproliferative disease may be treated by a therapeutic nucleic acid. A “therapeutic nucleic acid” is defined herein to refer to a nucleic acid which can be administered to a subject for the purpose of treating or preventing a disease. The nucleic acid is one which is known or suspected to be of benefit in the treatment of a hyperproliferative disease. Therapeutic benefit may arise, for example, as a result of alteration of expression of a particular gene or genes by the nucleic acid. Alteration of expression of a particular gene or genes may be inhibition or augmentation of expression of a particular gene.

In particular embodiments the therapeutic nucleic acid is in the form of a nucleic acid “expression construct”. Throughout this application, the term “expression construct” is meant to include any type of nucleic acid in which all or part of the nucleic acid is capable of being transcribed. The transcribed portion may encode a therapeutic gene capable of being translated into a therapeutic gene product such as a protein, but it need not be. In other embodiments the transcribed portion may simply act to inhibit or augment expression of a particular gene.

In certain embodiments of the present invention, the therapeutic nucleic acid encodes a “therapeutic gene”. As will be understood by those in the art, the term “therapeutic gene” includes genomic sequences, cDNA sequences, and smaller engineered gene segments that express, or may be adapted to express, proteins, polypeptides, domains, peptides, fusion proteins, and mutants, all of which are capable of providing a clinical benefit to a patient suffering from a hyperproliferative disease. The therapeutic nucleic acid encoding a therapeutic gene may comprise a contiguous nucleic acid sequence of about 5 to about 20,000 or more nucleotides, nucleosides, or base pairs.

In one embodiment, p53 gene therapy is contemplated. Human p53 gene therapy has been described in the literature since the mid-1990's. Roth et al. (1996) reported on retroviral-based therapy, and Clayman et al. (1998) described adenoviral delivery. U.S. Pat. Nos. 5,747,469, 6,017,524; 6,143,290; 6,410,010; and 6,511,847, U.S. Application 2002/0077313 and U.S. Application 2002/0006914 each describe methods of treating patients with p53, and are hereby incorporated by reference.

2. Therapeutic Methods and Assessment of Efficacy

Local, regional (together loco-regional) or systemic delivery of expression constructs to patients is contemplated. It is proposed that this approach will provide clinical benefit, defined broadly as any of the following: reducing primary tumor size, reducing occurrence or size of metastasis, reducing or stopping tumor growth, inducing remission, increasing the duration before recurrence, reducing tumor-associated pain, inhibiting tumor cell division, killing a tumor cell, inducing apoptosis in a tumor cell, reducing or eliminating tumor recurrence, and/or increasing patient survival.

Patients with unresectable tumors may be treated according to the present invention. As a consequence, the tumor may reduce in size, or the tumor vasculature may change such that the tumor becomes resectable. If so, standard surgical resection may be permitted. Another particular mode of administration that can be used in conjunction with surgery is treatment of an operative tumor bed. Thus, in either the primary gene therapy treatment, or in a subsequent treatment, one may perfuse the resected tumor bed with the vector during surgery, and following surgery, optionally by inserting a catheter into the surgery site.

A cancer recurrence may be defined as the reappearance or rediagnosis of a patent as having any cancer following one or more of surgery, radiotherapy or chemotherapy. The patient need not have been reported as disease free, but merely that the patient has exhibited renewed cancer growth following some degree of clinical response by the first therapy. The clinical response may be, but is not limited to, stable disease, tumor regression, tumor necrosis, or absence of demonstrable cancer.

In certain embodiments it may be advantageous to assess the efficacy of therapeutic treatment via diagnostic imaging. For example in some embodiments of the present invention a patient may be examined via diagnostic imaging before therapeutic treatment, during therapeutic treatment and after therapeutic treatment. In certain other instances the efficacy of therapeutic treatment may be measured by examining a patient via diagnostic imaging before and after such treatment. Other times the efficacy of therapeutic treatment may be measured during and after such treatment, or simply after the treatment. Of course, the number of times the efficacy of a therapeutic treatment is evaluated via diagnostic imaging may depend on the type of therapy or therapies used in treatment, the duration of the treatment, the patient's overall health as assessed by a clinician, the type of hyperproliferative disease or condition being treated or some combination thereof.

In particular embodiments, one such advantageous form of diagnostic imaging which may be used to assess the effacy of a particular therapy when treating a patient is positron emission tomography, also called PET imaging or a PET scan. A PET scan is a diagnostic examination that involves the acquisition of physiologic images based on the detection of radiation from the emission of positrons. Positrons are tiny particles emitted from a radioactive substance administered to the patient. The subsequent images of the human body developed with this technique are used to evaluate a variety of diseases. PET scans are used to detect cancer and to examine the effects of cancer therapy by characterizing biochemical changes in the cancer. These scans can be performed on the whole body or of discrete regions of the body. The present inventors have discovered that use of PET scans provides a more accurate assessment of tumor response to gene therapy than a traditional CAT scan.

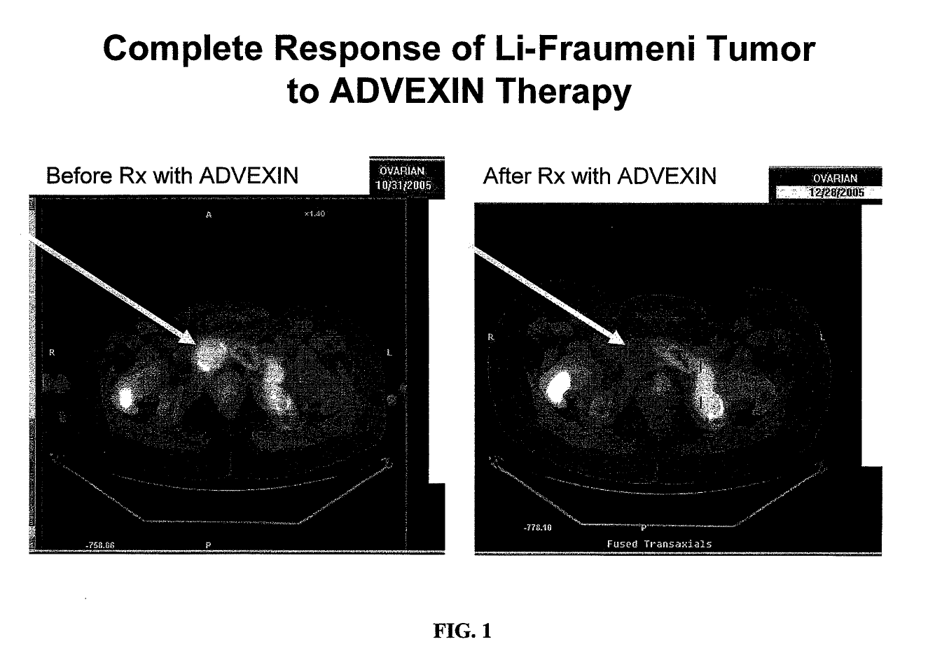

Before the examination begins, a radioactive substance is produced in a machine called a cyclotron and attached, or tagged, to a natural body compound, most commonly glucose, but sometimes water or ammonia. Once this substance is administered to the patient, the radioactivity localizes in the appropriate areas of the body and is detected by the PET scanner. Different colors or degrees of brightness on a PET image represent different levels of tissue or organ function. For example, because healthy tissue uses glucose for energy, it accumulates some of the tagged glucose, which will show up on the PET images. However, cancerous tissue, which uses more glucose than normal tissue, will accumulate more of the substance and appear brighter than normal tissue on the PET images. A nurse or technologist administers the radioactive substance via an intravenous injection (although in some cases, it will be given through an existing intravenous line or inhaled as a gas). It will then take approximately 30 to 90 minutes for the substance to travel through the body and accumulate in the tissue under study. FIG. 1 shows before and after treatment PET scans.

3. Hyperproliferative Diseases and Conditions to be Treated

Exemplary hyperproliferative lesions for which treatment is contemplated in the present invention include the following: Squamous cell carcinoma, basal cell carcinoma, adenoma, adenocarcinoma, linitis plastica, insulinoma, glucagonoma, gastrinoma, vipoma, cholangiocarcinoma, hepatocellular carcinoma, adenoid cystic carcinoma, carcinoid tumor, prolactinoma, oncocytoma, hurthle cell adenoma, renal cell carcinoma, endometrioid adenoma, cystadenoma, pseudomyxoma peritonei, Warthin's tumor, thymoma, thecoma, granulosa cell tumor, arrhenoblastoma, Sertoli-Leydig cell tumor, paraganglioma, pheochromocytoma, glomus tumor, melanoma, soft tissue sarcoma, desmoplastic small round cell tumor, fibroma, fibrosarcoma, myxoma, lipoma, liposarcoma, leiomyoma, leiomyosarcoma, myoma, myosarcoma, rhabdomyoma, rhabdomyosarcoma, pleomorphic adenoma, nephroblastoma, brenner tumor, synovial sarcoma, mesothelioma, dysgerminoma, germ cell tumors, embryonal carcinoma, yolk sac tumor, teratomas, dermoid cysts, choriocarcinoma, mesonephromas, hemangioma, angioma, hemangiosarcoma, angiosarcoma, hemangioendothelioma, hemangioendothelioma, Kaposi's sarcoma, hemangiopericytoma, lymphangioma, cystic lymphangioma, osteoma, osteosarcoma, osteochondroma, cartilaginous exostosis, chondroma, chondrosarcoma, giant cell tumors, Ewing's sarcoma, odontogenic tumors, cementoblastoma, ameloblastoma, craniopharyngioma gliomas mixed oligoastrocytomas, ependymoma, astrocytomas, glioblastomas, oligodendrogliomas, neuroepitheliomatous neoplasms, neuroblastoma, retinoblastoma, meningiomas, neurofibroma, neurofibromatosis, schwannoma, neurinoma, neuromas, granular cell tumors, alveolar soft part sarcomas, lymphomas, non-Hodgkin's lymphoma, lymphosarcoma, Hodgkin's disease, small lymphocytic lymphoma, lymphoplasmacytic lymphoma, mantle cell lymphoma, primary effusion lymphoma, mediastinal (thymic) large cell lymphoma, diffuse large B-cell lymphoma, intravascular large B-cell lymphoma, Burkitt lymphoma, splenic marginal zone lymphoma, follicular lymphoma, extranodal marginal zone B-cell lymphoma of mucosa-associated lymphoid tissue (MALT-lymphoma), nodal marginal zone B-cell lymphoma, mycosis fungoides, Sezary syndrome, peripheral T-cell lymphoma, angioimmunoblastic T-cell lymphoma, subcutaneous panniculitis-like T-cell lymphoma, anaplastic large cell lymphoma, hepatosplenic T-cell lymphoma, enteropathy type T-cell lymphoma, lymphomatoid papulosis, primary cutaneous anaplastic large cell lymphoma, extranodal NK/T cell lymphoma, blastic NK cell lymphoma, plasmacytoma, multiple myeloma, mastocytoma, mast cell sarcoma, mastocytosis,mast cell leukemia, langerhans cell histiocytosis, histiocytic sarcoma, langerhans cell sarcoma dendritic cell sarcoma, follicular dendritic cell sarcoma, Waldenstrom macroglobulinemia, lymphomatoid granulomatosis, acute leukemia, lymphocytic leukemia, acute lymphoblastic leukemia, acute lymphocytic leukemia, chronic lymphocytic leukemia, adult T-cell leukemia/lymphoma, plasma cell leukemia, T-cell large granular lymphocytic leukemia, B-cell prolymphocytic leukemia, T-cell prolymphocytic leukemia, pecursor B lymphoblastic leukemia, precursor T lymphoblastic leukemia, acute erythroid leukemia, lymphosarcoma cell leukemia, myeloid leukemia, myelogenous leukemia, acute myelogenous leukemia, chronic myelogenous leukemia, acute promyelocytic leukemia, acute promyelocytic leukemia, acute myelomonocytic leukemia, basophilic leukemia, eosinophilic leukemia, acute basophilic leukemia, acute myeloid leukemia, chronic myelogenous leukemia, monocytic leukemia, acute monoblastic and monocytic leukemia, acute megakaryoblastic leukemia, acute myeloid leukemia and myelodysplastic syndrome, chloroma or myeloid sarcoma, acute panmyelosis with myelofibrosis, hairy cell leukemia, juvenile myelomonocytic leukemia, aggressive NK cell leukemia, polycythemia vera, myeloproliferative disease, chronic idiopathic myelofibrosis, essential thrombocytemia, chronic neutrophilic leukemia, chronic eosinophilic leukemia/ hypereosinophilic syndrome, post-transplant lymphoproliferative disorder, chronic myeloproliferative disease, myelodysplastic/myeloproliferative diseases, chronic myelomonocytic leukemia and myelodysplastic syndrome.

A particular condition contemplated for treatment using the methods of the present invention is Li-Fraumeni Syndrome (LFS). LFS is a rare autosomal-dominant disease which is typically caused by mutations in TKP53 (p53). LFS is characterized by a predisposition to many kinds of cancers, the young onset of malignancies and the potential for multiple primary sites of cancer during the lifetime of affected individuals. For example, LFS patients with germline p53 mutations may have an increased susceptibility to colorectal cancer and present up to several decades earlier than the general population (Wong et al., 2006).

LFS was initially described in 1969 in a retrospective epidemiologic review of more than 600 pediatric sarcoma patients, but it was not until 1990 that it was demonstrated that germline abnormalities of the p53 tumor suppressor gene could account for the occurrence of cancer in many classic Li-Fraumeni families. Familial LFS is typically diagnosed by the presence of the following criteria: (1) a proband diagnosed with sarcoma when younger than 45 years, (2) a first-degree relative with any cancer diagnosed when younger than 45 years, and (3) another first- or second-degree relative of the same genetic lineage with any cancer diagnosed when younger than 45 years or sarcoma diagnosed at any age.

The cancers that occur most commonly in LFS are breast cancer, brain tumors, acute leukemia, soft tissue sarcomas, osteosarcoma, and adrenal cortical carcinoma. A significant proportion of affected patients, particularly children, can be treated successfully for the initial cancer but are at significant risk of subsequent development of a second primary malignancy.

Although mutations in p53 typically cause LFS, certain forms of LFS that lack germline p53 mutations have been shown to have germline mutations of the checkpoint kinase gene CHK2, which has also been linked to familial predisposition of early onset cancers. The lack of any demonstrable p53 mutations in a significant minority of LFS cases indicates that, in certain instances, other genes may contribute to the appearance of LFS. It would be straightforward to assess a Li Fraumeni patient to determine which type of mutation exists.

The present invention also contemplates using p53 gene therapy to treat Li Fraumeni patients. As shown in the example, the use of adenoviral delivery of p53 to a Li Fraumeni patient's tumor resulted in a dramatic response in which the tumor was completely eliminated. This constitutes the first treatment of a Li Fraumeni patient with p53 gene therapy. Many Li Fraumeni patients exhibit tumors of heterogeneous nature, and these have multiple abnormalities including but not limited to mutations in p53. Thus, it was not clear a priori that provision of a single gene therapy, i.e., p53, would provide a clinical benefit to these patients.

Successful treatment of patients with the Li-Fraumeni Syndrome is problematic due to the presence of multiple malignancies that share a common pathogenic defect fundamental to cancer progression and the development of treatment resistance. As the prototypical tumor suppressor gene, p53 is a transcription factor that controls several biological processes important for tumor suppression including regulation of the cell cycle, angiogenesis and apoptosis. The p53 tumor suppressor gene has been called the “Guardian of the Genome” because it normally halts progression through the cell cycle in the presence of DNA damage facilitating its repair or initiating apoptotic cell death when DNA repair is incomplete (Lane et al., 1992). Abnormal p53 function characteristic of Li-Fraumeni tumors results in uncontrolled cellular proliferation through loss of cell cycle regulation and treatment resistance from impaired apoptosis in response to DNA damaging radiation and chemotherapy.

These known mechanisms of p53 action provide insights into the clinical activity of adenoviral p53 gene therapy observed in this Li-Fraumeni patient and account for the ability of this treatment to induce stabilization of tumor growth or tumor regressions in other studies. In response to the presence of DNA damage from prior radiation or chemotherapy, cell cycle arrest is mediated through p53 activation of p21 which works through cyclin-dependent kinase pathways to block the transcription of genes that are required for entry into S-phase (El-Deiry et al., 1993; Harper et al., 1993; Xiong et al., 1993). Cells with irreparable DNA lesions may undergo apoptosis induced by a variety of apoptosis mediators including BAK and BAX (Lozano and Zambetti, 2005b). The induction of both p21 and BAK that are classical mediators of p53 cell cycle arrest and apoptosis pathways respectively have been demonstrated following p53 gene therapy in other clinical trials (Swisher et al., 2003). Tumor growth control with clinically stable disease may also be mediated by p53 activation of anti-angiogenic mechanisms. p53 has been shown to down regulate the expression of vascular endothelial growth factor (VEGF) and to activate the transcription of secretory inhibitors of angiogenesis (Dameron et al., 1994; Nishizaki et al., 1999). Hence, p53 gene therapy induces cell cycle arrest, cellular apoptotic pathways and anti-angiogenesis that can mediate both direct and bystander anti-tumor effects (Roth, 2006).

Correlation of the different types of p53 mutations in Li-Fraumeni tumors with distinct tumor histologies and related effects in transgenic animal models have provided important insights regarding the nature of p53 mutations commonly found in many familial and non-familial cancers. These findings may have importance regarding the effects of p53 gene therapy. Approximately 25% of Li-Fraumeni germ line mutations and non-familial p53 tumor abnormalities represent either nonsense, frameshift or splice mutations which are likely to result in a “null phenotype” with non-functional or absent p53 protein. The patient in this study has this type of a frameshift mutation resulting in a downstream stop codon and a null p53 phenotype. In these null phenotype tumors, the activity of p53 administered by gene therapy would not be opposed by any competing mutated p53 with “gain of function” activity. The remaining 75% of Li-Fraumeni and non-familial somatic p53 tumor abnormalities are missense mutations that may alter p53 transcriptional activity and result in p53 gain of function mutations that can effect tumor pathophysiology. These mutation types have been classified into subgroups depending upon the codons where the mutations are found (e.g., sites of transcriptional activity binding to major and minor grooves of DNA etc. (Olivier et al., 2003). Further studies are contemplated to determine whether these “gain of function” mutations may alter the efficacy of wild-type p53 provided by gene transfer.

Another important element of the below examples is that p53 gene therapy was well tolerated without significant side effects consistent with clinical experience in other patients. The absence of genotoxic side effects associated with p53 gene therapy may be particularly important for the treatment of Li-Fraumeni patients who are predisposed to develop secondary malignancies following the administration of conventional DNA damaging radiation and chemotherapy. In addition, the absence of cross reacting side effects permits the combined use of p53 gene therapy with other cancer therapeutics that may have synergistic effects with contusugene.

The Li-Fraumeni patient treated in the below example had a pre-treatment phenotype predictive of ADVEXIN activity: p14ARF+, HDM2 low, BCL2 low and CAR+and post treatment activation of cell cycle arrest (p21) and apoptotic pathways (cleaved caspase 3). Interestingly, CAR expression increased significantly after ADVEXIN administration as did HDM2 levels and p14ARF. BCL2 levels were decreased.

These observations have important implications regarding optimizing therapy with adenoviruses in general and with Advexin in particular. The benefits of adenoviral tumor therapy may be enhanced by first administration of ADVEXIN followed by subsequent Ad vector administration timed to the maximum increase in CAR expression. Related timing issues may also be important for the down regulation of BCL2 with respect to the administration of subsequent therapies that are mediated by apoptosis (e.g., chemo, XRT etc.). Thus, the expected increase in HDM2 mediated by wild-type p53 implies that a combination vector with p53 and siRNA to inhibit HDM2 would enhance the therapeutic effects of ADVEXIN.

II. Primary Therapies

A. Therapeutic Nucleic Acids

Certain embodiments of the present invention concern the administration of a therapeutic nucleic acid. The term “nucleic acid” is well known in the art. A “nucleic acid” as used herein will generally refer to a molecule (i.e., a strand) of DNA, RNA or a derivative or analog thereof, comprising a nucleotide base. A nucleotide base includes, for example, a naturally occurring purine or pyrimidine base found in DNA (e.g., an adenine “A,” a guanine “G,” a thymine “T” or a cytosine “C” or RNA (e.g., an A, a G, an uracil “U” or a C). The term “nucleic acid” encompass the terms “oligonucleotide” and “polynucleotide,” each as a subgenus of the term “nucleic acid.” The term “oligonucleotide” refers to a molecule of between about 8 and about 100 nucleotide bases in length. The term “polynucleotide” refers to at least one molecule of greater than about 100 nucleotide bases in length.

In certain embodiments, a “gene” refers to a nucleic acid that is transcribed. In certain aspects, the gene includes regulatory sequences involved in transcription or message production. In particular embodiments, a gene comprises transcribed sequences that encode for a protein, polypeptide or peptide. As will be understood by those in the art, this functional term “gene” includes genomic sequences, RNA or cDNA sequences or smaller engineered nucleic acid segments, including nucleic acid segments of a non-transcribed part of a gene, including but not limited to the non-transcribed promoter or enhancer regions of a gene. Smaller engineered nucleic acid segments may express, or may be adapted to express proteins, polypeptides, polypeptide domains, peptides, fusion proteins, mutant polypeptides and/or the like.

“Isolated substantially away from other coding sequences” means that the gene of interest forms part of the coding region of the nucleic acid segment, and that the segment does not contain large portions of naturally-occurring coding nucleic acid, such as large chromosomal fragments or other functional genes or cDNA coding regions. Of course, this refers to the nucleic acid as originally isolated, and does not exclude genes or coding regions later added to the nucleic acid by the hand of man.

1. Therapeutic Nucleic Acids Encoding Therapeutic Genes

As discussed above, within various embodiments of the present invention there may be a need to provide a patient with a therapeutic gene for the purposes of treating a hyperproliferative disease. The term “gene therapy” within this application can be defined as delivery of a therapeutic gene or other therapeutic nucleic acid to a patient in need of such for purposes of treating a hyperproliferative disease or for treating a condition which, if left untreated may result in a hyperproliferative disease. Encompassed within the definition of “therapeutic gene” is a “biologically functional equivalent” therapeutic gene. Accordingly, sequences that have about 70% to about 99% homology of amino acids that are identical or functionally equivalent to the amino acids of the therapeutic gene will be sequences that are biologically functional equivalents provided the biological activity of the protein is maintained. Classes of therapeutic genes include tumor suppressor genes, cell cycle regulators, pro-apoptotic genes, cytokines, toxins, anti-angiogenic factors, and molecules than inhibit oncogenes, pro-angiogenic factors, growth factors, antisense transcripts, rybozymes and RNAi.

Examples of therapeutic genes include, but are not limited to, Rb, CFTR, p16, p21, p27, p57, p73, C-CAM, APC, CTS-1, zac1, scFV ras, DCC, NF-1, NF-2, WT-1, MEN-I, MEN-II, BRCA1, VHL, MMAC1, FCC, MCC, BRCA2, IL-1, IL-2, IL-3, IL-4, IL-5, IL-6, IL-7, IL-8, IL-9, IL-10, IL-11 IL-12, GM-CSF, G-CSF, thymidine kinase, mda7, fus, interferon α, interferon β, interferon γ, ADP, p53, ABLI, BLC1, BLC6, CBFA1, CBL, CSFIR, ERBA, ERBB, EBRB2, ETS1, ETS2, ETV6, FGR, FOX, FYN, HCR, HRAS, JUN, KRAS, LCK, LYN, MDM2, MLL, MYB, MYC, MYCL1, MYCN, NRAS, PIM1, PML, RET, SRC, TAL1, TCL3, YES, MADH4, RB1, TP53, WT1, TNF, BDNF, CNTF, NGF, IGF, GMF, aFGF, bFGF, NT3, NT5, ApoAI, ApoAIV, ApoE, Rap1A, cytosine deaminase, Fab, ScFv, BRCA2, zac1, ATM, HIC-1, DPC-4, FHIT, PTEN, ING1, NOEY1, NOEY2, OVCA1, MADR2, 53BP2, IRF-1, Rb, zac1, DBCCR-1, rks-3, COX-1, TFPI, PGS, Dp, E2F, ras, myc, neu, raf, erb, fms, trk, ret, gsp, hst, abl, E1A, p300, VEGF, FGF, thrombospondin, BAI-1, GDAIF, or MCC.

Other examples of therapeutic genes include genes encoding enzymes. Examples include, but are not limited to, ACP desaturase, an ACP hydroxylase, an ADP-glucose pyrophorylase, an ATPase, an alcohol dehydrogenase, an amylase, an amyloglucosidase, a catalase, a cellulase, a cyclooxygenase, a decarboxylase, a dextrinase, an esterase, a DNA polymerase, an RNA polymerase, a hyaluron synthase, a galactosidase, a glucanase, a glucose oxidase, a GTPase, a helicase, a hemicellulase, a hyaluronidase, an integrase, an invertase, an isomerase, a kinase, a lactase, a lipase, a lipoxygenase, a lyase, a lysozyme, a pectinesterase, a peroxidase, a phosphatase, a phospholipase, a phosphorylase, a polygalacturonase, a proteinase, a peptidease, a pullanase, a recombinase, a reverse transcriptase, a topoisomerase, a xylanase, a reporter gene, an interleukin, or a cytokine.

Further examples of therapeutic genes include the gene encoding carbamoyl synthetase I, omithine transcarbamylase, arginosuccinate synthetase, arginosuccinate lyase, arginase, fumarylacetoacetate hydrolase, phenylalanine hydroxylase, alpha-i antitrypsin, glucose-6-phosphatase, low-density-lipoprotein receptor, porphobilinogen deaminase, factor VIII, factor IX, cystathione β-synthase, branched chain ketoacid decarboxylase, albumin, isovaleryl-CoA dehydrogenase, propionyl CoA carboxylase, methyl malonyl CoA mutase, glutaryl CoA dehydrogenase, insulin, β-glucosidase, pyruvate carboxylase, hepatic phosphorylase, phosphorylase kinase, glycine decarboxylase, H-protein, T-protein, Menkes disease copper-transporting ATPase, Wilson's disease copper-transporting ATPase, cytosine deaminase, hypoxanthine-guanine phosphoribosyltransferase, galactose- 1-phosphate uridyltransferase, phenylalanine hydroxylase, glucocerbrosidase, sphingomyelinase, α-L-iduronidase, glucose-6-phosphate dehydrogenase, HSV thymidine kinase, or human thymidine kinase.

Therapeutic genes also include genes encoding hormones. Examples include, but are not limited to, genes encoding growth hormone, prolactin, placental lactogen, luteinizing hormone, follicle-stimulating hormone, chorionic gonadotropin, thyroid-stimulating hormone, leptin, adrenocorticotropin, angiotensin I, angiotensin II, β-endorphin, β-melanocyte stimulating hormone, cholecystokinin, endothelin I, galanin, gastric inhibitory peptide, glucagon, insulin, lipotropins, neurophysins, somatostatin, calcitonin, calcitonin gene related peptide, β-calcitonin gene related peptide, hypercalcemia of malignancy factor, parathyroid hormone-related protein, parathyroid hormone-related protein, glucagon-like peptide, pancreastatin, pancreatic peptide, peptide YY, PHM, secretin, vasoactive intestinal peptide, oxytocin, vasopressin, vasotocin, enkephalinamide, metorphinamide, α melanocyte stimulating hormone, atrial natriuretic factor, amylin, amyloid P component, corticotropin releasing hormone, growth hormone releasing factor, luteinizing hormone-releasing hormone, neuropeptide Y, substance K, substance P, or thyrotropin releasing hormone.

Other examples of therapeutic genes include genes encoding antigens present in hyperproliferative tissues that can be used to elicit and immune response against that tissue. Anti-cancer immune therapies are well known in the art, for example, in greater detail in PCT application WO0333029, WO0208436, WO0231168, and WO0285287, each of which is specifically incorporated by reference in its entirety.

Yet other therapeutic genes are those that encode inhibitory molecules, such as antisense, ribozymes, siRNA and single chain antibodies. Such molecules can be used advantageously to inhibit hyperproliferative genes, such as oncogenes, inducers of cellular proliferation and pro-angiogenic factors.

### a. Nucleic Acids Encoding Tumor Suppressors

A “tumor suppressor” refers to a polypeptide that, when present in a cell, reduces the tumorigenicity, malignancy, or hyperproliferative phenotype of the cell. The nucleic acid sequences encoding tumor suppressor gene amino acid sequences include both the full length nucleic acid sequence of the tumor suppressor gene, as well as non-full length sequences of any length derived from the full length sequences. It being further understood that the sequence includes the degenerate codons of the native sequence or sequences which may be introduced to provide codon preference in a specific host cell.

“Tumor suppressor genes” are generally defined herein to refer to nucleic acid sequences that reduce the tumorigenicity, malignancy, or hyperproliferative phenotype of the cell. Thus, the absence, mutation, or disruption of normal expression of a tumor suppressor gene in an otherwise healthy cell increases the likelihood of, or results in, the cell attaining a neoplastic state. Conversely, when a functional tumor suppressor gene or protein is present in a cell, its presence suppresses the tumorigenicity, malignancy or hyperproliferative phenotype of the host cell. Examples of tumor suppressor nucleic acids within this definition include, but are not limited to APC, CYLD, HIN-1, KRAS2b, p16, p19, p21, p27, p27mt, p53, p57, p73, PTEN, Rb, Uteroglobin, Skp2, BRCA-1, BRCA-2, CHK2, CDKN2A, DCC, DPC4, MADR2/JV18, FHIT, MEN1, MEN2, MTS1, NF1, NF2, VHL, WRN, WT1, CFTR, C-CAM, CTS-1, zac1, scFV, ras, MMAC1, FCC, MCC, Gene 26 (CACNA2D2), PL6, Beta* (BLU), Luca-1 (HYAL1), Luca-2 (HYAL2), 123F2 (RASSF1), 101F6, Gene 21 (NPRL2), or a gene encoding a SEM A3 polypeptide and FUS 1. Other exemplary tumor suppressor genes are described in a database of tumor suppressor genes at (www.cise.ufl.edu/˜yy1/HTML-TSGDB/Homepage.html), incorporated therein by reference. This database is herein specifically incorporated by reference into this and all other sections of the present application. Nucleic acids encoding tumor suppressor genes, as discussed above, include tumor suppressor genes, or nucleic acids derived therefrom (e.g., cDNAs, cRNAs, mRNAs, and subsequences thereof encoding active fragments of the respective tumor suppressor amino acid sequences), as well as vectors comprising these sequences. One of ordinary skill in the art would be familiar with tumor suppressor genes that can be applied in the present invention.

p53, one of the best known tumor suppressors, is phosphoprotein of about 390 amino acids which can be subdivided into four domains: (i) a highly charged acidic region of about 75-80 residues, (ii) a hydrophobic proline-rich domain (position 80 to 150), (iii) a central region (from 150 to about 300), and (iv) a highly basic C-terminal region. The sequence of p53 is well conserved in vertebrate species, but there have been no proteins homologous to p53 identified in lower eucaryotic organisms. Comparisons of the amino acid sequence of human, African green monkey, golden hamster, rat, chicken, mouse, rainbow trout and Xenopus laevis p53 proteins indicated five blocks of highly conserved regions, which coincide with the mutation clusters found in p53 in human cancers evolution.

p53 is located in the nucleus of cells and is very labile. Agents which damage DNA induce p53 to become very stable by a post-translational mechanism, allowing its concentration in the nucleus to increase dramatically. p53 suppresses progression through the cell cycle in response to DNA damage, thereby allowing DNA repair to occur before replicating the genome. Hence, p53 prevents the transmission of damaged genetic information from one cell generation to the next initiates apoptosis if the damage to the cell is severe. Mediators of this effect included Bax, a well-known “inducer of apoptosis.”

As discussed above, mutations in p53 can cause cells to become oncogenically transformed, and transfection studies have shown that p53 acts as a potent transdominant tumor suppressor, able to restore some level of normal growth to cancerous cells in vitro. p53 is a potent transcription factor and once activated, it represses transcription of one set of genes, several of which are involved in stimulating cell growth, while stimulating expression of other genes involved in cell cycle control.

### b. Nucleic Acids Encoding Single Chain Antibodies

In certain embodiments of the present invention, the nucleic acid of the pharmaceutical compositions and devices set forth herein encodes a single chain antibody. Single-chain antibodies are described in U.S. Pat. Nos. 4,946,778 and 5,888,773, each of which are hereby incorporated by reference.

### c. Nucleic Acids Encoding Cytokines

The term “cytokine” is a generic term for proteins released by one cell population which act on another cell as intercellular mediators. A “cytokine amino acid sequence” refers to a polypeptide that, when present in a cell, maintains some or all of the function of a cytokine. The nucleic acid sequences encoding cytokine amino acid sequences include both the full length nucleic acid sequence of the cytokine, as well as non-full length sequences of any length derived from the full length sequences. It being further understood, as discussed above, that the sequence includes the degenerate codons of the native sequence or sequences which may be introduced to provide codon preference in a specific host cell.

Examples of such cytokines are lymphokines, monokines, growth factors and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; prostaglandin, fibroblast growth factor; prolactin; placental lactogen, OB protein; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β.; platelet-growth factor; transforming growth factors (TGFs) such as TGF-α and TGF-β; insulin-like growth factor-I and -II; erythropoietin (EPO); osteoinductive factors; interferons such as interferon-α, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1α, IL-2, IL-3, IL-4, IL-5, IL-6, IL-7, IL-8, IL-9, IL-11, IL-12; IL-13, IL-14, IL-15, IL-16, IL-17, IL-18, LIF, G-CSF, GM-CSF, M- CSF, EPO, kit-ligand or FLT-3.

Another example of a cytokine is IL-10. IL-10 is a pleiotropic homodimeric cytokine produced by immune system cells, as well as some tumor cells (Ekmekcioglu et al., 1999). Its immunosuppressive function includes potent inhibition of proinflammatory cytokine synthesis, including that of IFNγ, TNFα, and IL-6 (De Waal Malefyt et al., 1991). The family of IL-10-like cytokines is encoded in a small 195 kb gene cluster on chromosome 1q32, and consists of a number of cellular proteins (IL- 10, IL- 19, IL-20, MDA-7) with structural and sequence homology to IL-10 (Kotenko et al., 2000; Gallagher et al., 2000; Blumberg et al., 2001; Dumoutier et al., 2000; Knapp et al., 2000; Jiang et al., 1995; Jiang et al., 1996).

A recently discovered putative member of the cytokine family is MDA-7. MDA-7 has been characterized as an IL-10 family member and is also known as IL-24. Chromosomal location, transcriptional regulation, murine and rat homologue expression, and putative protein structure all allude to MDA-7 being a cytokine (Knapp et al., 2000; Schaefer et al., 2000; Soo et al., 1999; Zhang et al., 2000). Similar to GM-CSF, TNFα, and IFNγ transcripts, all of which contain AU-rich elements in their 3'UTR targeting mRNA for rapid degradation, MDA-7 has three AREs in its 3'UTR. Mda-7 mRNA has been identified in human PBMC (Ekmekcioglu, et al., 2001), and although no cytokine function of human MDA-7 protein has been previously reported, MDA-7 has been designated as IL-24 based on the gene and protein sequence characteristics (NCBI database accession XM_{—}001405).

### d. Nucleic Acids Encoding Pro-Apoptotic Genes/Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., BCl_{XL}, Bcl_{W}, Bcl_{S}, Mcl-1, A1, Bfl-1) or counteract Bcl-2 function and promote cell death. The latter, known as pro-apoptotic genes, encode proteins that induce or sustain apoptosis to an active form. The present invention contemplates inclusion of any pro-apoptotic gene amino acid sequence known to those of ordinary skill in the art. Exemplary pro-apoptotic genes include CD95, caspase-3, Bax, Bag-1, CRADD, TSSC3, bax, hid, Bak, MKP-7, PERP, bad, bcl-2, MST1, bbc3, Sax, BIK, BID, and mda7. One of ordinary skill in the art would be familiar with pro-apoptotic genes, and other such genes not specifically set forth herein that can be applied in the methods and compositions of the present invention.

Nucleic acids encoding pro-apoptotic gene amino acid sequences include pro-apoptotic genes, or nucleic acids derived therefrom (e.g., cDNAs, cRNAs, mRNAs, and subsequences thereof encoding active fragments of the respective pro-apoptotic amino acid sequence), as well as vectors comprising these sequences. A “pro-apoptotic gene amino acid sequence” refers to a polypeptide that, when present in a cell, induces or promotes apoptosis.

### e. Nucleic Acids Encoding Inhibitors of Angiogenesis

Inhibitors of angiogenesis include angiostatin and endostatin. Angiostatin is a polypeptide of approximately 200 amino acids. It is produced by the cleavage of plasminogen, a plasma protein that is important for dissolving blood clots. Angiostatin binds to subunits of ATP synthase exposed at the surface of the cell embedded in the plasma membrane. (Before this recent discovery, ATP synthase was known only as a mitochondrial protein. Endostatin is a polypeptide of 184 amino acids. It is the globular domain found at the C-terminus of Type XVIII (Mulder et al., 1995) collagen, a collagen found in blood vessels, cut off from the parent molecule.

Inhibitors of angiogenesis also include inhibitors or pro-angiongenic factors, such as antisense, ribozymes, siRNAs and single-chain antibodies, which are described elsewhere in this document. Epithelial cells express transmembrane proteins on their surface, called integrins, by which they anchor themselves to the extracellular matrix. It turns out that the new blood vessels in tumors express a vascular integrin, designated αv/β3, that is not found on the old blood vessels of normal tissues. Vitaxin®, a monoclonal antibody directed against the αv/β3 vascular integrin, shrinks tumors in mice without harming them. In Phase II clinical trials in humans, Vitaxin has shown some promise in shrinking solid tumors without harmful side effects.

### f. Nucleic Acids Encoding Inducers of Cellular Proliferation

The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally-occurring oncogenic growth factor.

The proteins FMS, ErbA, ErbB and neu are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

The proteins Jun, Fos and Myc are proteins that directly exert their effects on nuclear functions as transcription factors.

Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject. In other embodiment of the present invention, it is contemplated that siRNA, ribozyme and single-chain antibody therapies directed at particular inducers of cellular proliferation can be used to prevent expression of the inducer of cellular proliferation, and hence provide a clinical benefit to a cancer patient.

2. Additional Nucleic Acid Based Therapies

### a. Antisense

Antisense constructs may be designed to bind to the promoter and other control regions, exons, introns or even exon-intron boundaries of a gene. It is contemplated that the most effective antisense constructs will include regions complementary to intron/exon splice junctions. Thus, it is proposed that a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme; see below) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

### b. Ribozymes

In certain embodiments of the present invention, the nucleic acid of the pharmaceutical compositions and devices set forth herein is a ribozyme. Although proteins traditionally have been used for catalysis of nucleic acids, another class of macromolecules has emerged as useful in this endeavor. Ribozymes are RNA-protein complexes that cleave nucleic acids in a site-specific fashion. Ribozymes have specific catalytic domains that possess endonuclease activity (Kim and Cook, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Cook et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989; Cook et al., 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990). Recently, it was reported that ribozymes elicited genetic changes in some cells lines to which they were applied; the altered genes included the oncogenes H-ras, c-fos and genes of HIV. Most of this work involved the modification of a target mRNA, based on a specific mutant codon that is cleaved by a specific ribozyme.

### c. RNAi

In certain embodiments of the present invention, the therapeutic nucleic acid of the pharmaceutical compositions set forth herein is an RNAi. RNA interference (also referred to as “RNA-mediated interference” or RNAi) is a mechanism by which gene expression can be reduced or eliminated. Double-stranded RNA (dsRNA) has been observed to mediate the reduction, which is a multi-step process. dsRNA activates post-transcriptional gene expression surveillance mechanisms that appear to function to defend cells from virus infection and transposon activity (Fire et al., 1998; Grishok et al., 2000; Ketting et al., 1999; Lin and Avery et al., 1999; Montgomery et al., 1998; Sharp and Zamore, 2000; Tabara et al., 1999). Activation of these mechanisms targets mature, dsRNA-complementary mRNA for destruction. RNAi offers major experimental advantages for study of gene function. These advantages include a very high specificity, ease of movement across cell membranes, and prolonged down-regulation of the targeted gene (Fire et al., 1998; Grishok et al., 2000; Ketting et al., 1999; Lin and Avery et al., 1999; Montgomery et al., 1998; Sharp et al., 1999; Sharp and Zamore, 2000; Tabara et al., 1999). Moreover, dsRNA has been shown to silence genes in a wide range of systems, including plants, protozoans, fungi, C. elegans, Trypanasoma, Drosophila, and mammals (Grishok et al., 2000; Sharp et al., 1999; Sharp and Zamore, 2000; Elbashir et al., 2001). It is generally accepted that RNAi acts post-transcriptionally, targeting RNA transcripts for degradation. It appears that both nuclear and cytoplasmic RNA can be targeted (Bosher and Labouesse, 2000).

The endoribonuclease Dicer is known to produce two types of small regulatory RNAs that regulate gene expression: small interfering RNAs (siRNAs) and microRNAs (miRNAs) (Bernstein et al., 2001; Grishok et al., 2001; Hutvgner et al., 2001; Ketting et al., 2001; Knight and Bass, 2001). In animals, siRNAs direct target mRNA cleavage (Elbashir et al., 2001), whereas miRNAs block target mRNA translation (Lee et al., 1993; Reinhart et al., 2000; Brennecke et al., 2003; Xu et al., 2003). Recent data suggest that both siRNAs and miRNAs incorporate into similar perhaps even identical protein complexes, and that a critical determinant of mRNA destruction versus translation regulation is the degree of sequence complementary between the small RNA and its mRNA target (Hutvgner and Zamore, 2002; Mourelatos et al., 2002; Zeng et al., 2002; Doench et al., 2003; Saxena et al., 2003; Zeng et al., 2003). Many known miRNA sequences and their position in genomes or chromosomes can be found at www.sanger.ac.uk/Software/Rfam/mirna/help/summary.shtml.

siRNAs must be designed so that they are specific and effective in suppressing the expression of the genes of interest. Methods of selecting the target sequences, i.e., those sequences present in the gene or genes of interest to which the siRNAs will guide the degradative machinery, are directed to avoiding sequences that may interfere with the siRNA's guide function while including sequences that are specific to the gene or genes. Typically, siRNA target sequences of about 21 to 23 nucleotides in length are most effective. This length reflects the lengths of digestion products resulting from the processing of much longer RNAs as described above (Montgomery et al., 1998).

The making of siRNAs has been mainly through direct chemical synthesis; through processing of longer, double-stranded RNAs through exposure to Drosophila embryo lysates; or through an in vitro system derived from S2 cells. Use of cell lysates or in vitro processing may further involve the subsequent isolation of the short, 21-23 nucleotide siRNAs from the lysate, etc., making the process somewhat cumbersome and expensive. Chemical synthesis proceeds by making two single stranded RNA-oligomers followed by the annealing of the two single stranded oligomers into a double-stranded RNA. Methods of chemical synthesis are diverse. Non-limiting examples are provided in U.S. Pat. Nos. 5,889,136, 4,415,723, and 4,458,066, expressly incorporated herein by reference, and in Wincott et al. (1995).

Several further modifications to siRNA sequences have been suggested in order to alter their stability or improve their effectiveness. It is suggested that synthetic complementary 21-mer RNAs having di-nucleotide overhangs (i.e., 19 complementary nucleotides+3′ non-complementary dimers) may provide the greatest level of suppression. These protocols primarily use a sequence of two (2′-deoxy) thymidine nucleotides as the di-nucleotide overhangs. These dinucleotide overhangs are often written as dTdT to distinguish them from the typical nucleotides incorporated into RNA. The literature has indicated that the use of dT overhangs is primarily motivated by the need to reduce the cost of the chemically synthesized RNAs. It is also suggested that the dTdT overhangs might be more stable than UU overhangs, though the data available shows only a slight (<20%) improvement of the dTdT overhang compared to an siRNA with a UU overhang.

Chemically synthesized siRNAs are found to work optimally when they are in cell culture at concentrations of 25-100 nM, but concentrations of about 100 nM have achieved effective suppression of expression in mammalian cells. siRNAs have been most effective in mammalian cell culture at about 100 nM. In several instances, however, lower concentrations of chemically synthesized siRNA have been used (Caplen et al., 2000; Elbashir et al., 2001).

WO 99/32619 and WO 01/68836 suggest that RNA for use in siRNA may be chemically or enzymatically synthesized. Both of these texts are incorporated herein in their entirety by reference. The enzymatic synthesis contemplated in these references is by a cellular RNA polymerase or a bacteriophage RNA polymerase (e.g., T3, T7, SP6) via the use and production of an expression construct as is known in the art. For example, see U.S. Pat. No. 5,795,715. The contemplated constructs provide templates that produce RNAs that contain nucleotide sequences identical to a portion of the target gene. The length of identical sequences provided by these references is at least 25 bases, and may be as many as 400 or more bases in length. An important aspect of this reference is that the authors contemplate digesting longer dsRNAs to 21-25mer lengths with the endogenous nuclease complex that converts long dsRNAs to siRNAs in vivo. They do not describe or present data for synthesizing and using in vitro transcribed 21-25mer dsRNAs. No distinction is made between the expected properties of chemical or enzymatically synthesized dsRNA in its use in RNA interference.

Similarly, WO 00/44914, incorporated herein by reference, suggests that single strands of RNA can be produced enzymatically or by partial/total organic synthesis. Preferably, single-stranded RNA is enzymatically synthesized from the PCR products of a DNA template, preferably a cloned cDNA template and the RNA product is a complete transcript of the cDNA, which may comprise hundreds of nucleotides. WO 01/36646, incorporated herein by reference, places no limitation upon the manner in which the siRNA is synthesized, providing that the RNA may be synthesized in vitro or in vivo, using manual and/or automated procedures. This reference also provides that in vitro synthesis may be chemical or enzymatic, for example using cloned RNA polymerase (e.g., T3, T7, SP6) for transcription of the endogenous DNA (or cDNA) template, or a mixture of both. Again, no distinction in the desirable properties for use in RNA interference is made between chemically or enzymatically synthesized siRNA.

U.S. Pat. No. 5,795,715 reports the simultaneous transcription of two complementary DNA sequence strands in a single reaction mixture, wherein the two transcripts are immediately hybridized. The templates used are preferably of between 40 and 100 base pairs, and which is equipped at each end with a promoter sequence. The templates are preferably attached to a solid surface. After transcription with RNA polymerase, the resulting dsRNA fragments may be used for detecting and/or assaying nucleic acid target sequences.

U.S. Patent App. 20050203047 reports of a method of modulating gene expression through RNA interference by incorporating a siRNA or miRNA sequence into a transfer RNA (tRNA) encoding sequence. The tRNA containing the siRNA or miRNA sequence may be incorporated into a nucleic acid expression construct so that this sequence is spliced from the expressed tRNA. The siRNA or miRNA sequence may be positioned within an intron associated with an unprocessed tRNA transcript, or may be positioned at either end of the tRNA transcript.

B. Preparation of Nucleic Acids

A nucleic acid may be made by any technique known to one of ordinary skill in the art, such as for example, chemical synthesis, enzymatic production or biological production. Non-limiting examples of a synthetic nucleic acid (e.g., a synthetic oligonucleotide), include a nucleic acid made by in vitro chemical synthesis using phosphotriester, phosphite or phosphoramidite chemistry and solid phase techniques such as described in EP 266 032, incorporated herein by reference, or via deoxynucleoside H-phosphonate intermediates as described by Froehler et al. (1986) and U.S. Pat. No. 5,705,629, each incorporated herein by reference. Various mechanisms of oligonucleotide synthesis may be used, such as those methods disclosed in, U.S. Pat. Nos. 4,659,774; 4,816,571; 5,141,813; 5,264,566; 4,959,463; 5,428,148; 5,554,744; 5,574,146; 5,602,244 each of which are incorporated herein by reference.

A non-limiting example of an enzymatically produced nucleic acid include nucleic acids produced by enzymes in amplification reactions such as PCRTM (see for example, U.S. Pat. Nos. 4,683,202 and 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 2001, incorporated herein by reference).

C. Multigene Constructs and IRES

In certain embodiments of the invention, the use of internal ribosome binding sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

D. Purification of Nucleic Acids

A nucleic acid may be purified on polyacrylamide gels, cesium chloride centrifugation gradients, column chromatography or by any other means known to one of ordinary skill in the art (see for example, Sambrook et al., 2001, incorporated herein by reference). In certain aspects, the present invention concerns a nucleic acid that is an isolated nucleic acid. As used herein, the term “isolated nucleic acid” refers to a nucleic acid molecule (e.g., an RNA or DNA molecule) that has been isolated free of, or is otherwise free of, bulk of cellular components or in vitro reaction components, and/or the bulk of the total genomic and transcribed nucleic acids of one or more cells. Methods for isolating nucleic acids (e.g., equilibrium density centrifugation, electrophoretic separation, column chromatography) are well known to those of skill in the art.

III. Expression of Nucleic Acids

In accordance with the present invention, it will be desirable to produce therapeutic proteins in a target cell. Expression typically requires that appropriate signals be provided in the vectors or expression cassettes, and which include various regulatory elements, such as enhancers/promoters from viral and/or mammalian sources that drive expression of the genes of interest in host cells. Elements designed to optimize messenger RNA stability and translatability in host cells may also be included. Drug selection markers may be incorporated for establishing permanent, stable cell clones.

Viral vectors are selected eukaryotic expression systems. Included are adenoviruses, adeno-associated viruses, retroviruses, herpesviruses, lentivirus and poxviruses including vaccinia viruses and papilloma viruses including SV40. Viral vectors may be replication-defective, conditionally-defective or replication-competent. Also contemplated are non-viral delivery systems, including lipid-based vehicles.

A. Vectors and Expression Constructs

The term “vector” is used to refer to a carrier nucleic acid molecule into which a nucleic acid sequence can be inserted for introduction into a cell where it can be replicated and/or expressed. A nucleic acid sequence can be “exogenous” or “heterologous” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Sambrook et al., 2001 and Ausubel et al., 1996, both incorporated herein by reference).

The term “expression vector” refers to any type of genetic construct comprising a nucleic acid coding for a RNA capable of being transcribed. In some cases, RNA molecules are then translated into a protein, polypeptide, or peptide. Expression vectors can contain a variety of “control sequences,” which refer to nucleic acid sequences necessary for the transcription and possibly translation of an operable linked coding sequence in a particular host cell. In addition to control sequences that govern transcription and translation, vectors and expression vectors may contain nucleic acid sequences that serve other functions as well, as described below.

In order to express p53, it is necessary to provide an expression vector. The appropriate nucleic acid can be inserted into an expression vector by standard subcloning techniques. The manipulation of these vectors is well known in the art. Examples of fusion protein expression systems are the glutathione S-transferase system (Pharmacia, Piscataway, N.J.), the maltose binding protein system (NEB, Beverley, Mass.), the FLAG system (IBI, New Haven, Conn.), and the 6xHis system (Qiagen, Chatsworth, Calif.).

In yet another embodiment, the expression system used is one driven by the baculovirus polyhedron promoter. The gene encoding the protein can be manipulated by standard techniques in order to facilitate cloning into the baculovirus vector. A preferred baculovirus vector is the pBlueBac vector (Invitrogen, Sorrento, Calif.). The vector carrying the gene of interest is transfected into Spodoptera frugiperda (Sf9) cells by standard protocols, and the cells are cultured and processed to produce the recombinant protein. Mammalian cells exposed to baculoviruses become infected and may express the foreign gene only. This way one can transduce all cells and express the gene in dose dependent manner.

There also are a variety of eukaryotic vectors that provide a suitable vehicle in which recombinant polypeptide can be produced. HSV has been used in tissue culture to express a large number of exogenous genes as well as for high level expression of its endogenous genes. For example, the chicken ovalbumin gene has been expressed from HSV using an α promoter. Herz and Roizman (1983). The lacZ gene also has been expressed under a variety of HSV promoters.

Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. Thus, in certain embodiments, expression includes both transcription of a gene and translation of a RNA into a gene product. In other embodiments, expression only includes transcription of the nucleic acid.

In preferred embodiments, the nucleic acid is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription.

The particular promoter that is employed to control the expression of a nucleic acid is not believed to be critical, so long as it is capable of expressing the nucleic acid in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter.

In various other embodiments, the human cytomegalovirus (CMV) immediate early gene promoter, the SV40 early promoter and the Rous sarcoma virus long terminal repeat can be used to obtain high-level expression of transgenes. The use of other viral or mammalian cellular or bacterial phage promoters which are well-known in the art to achieve expression of a transgene is contemplated as well, provided that the levels of expression are sufficient for a given purpose.

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Additionally any promoter/enhancer combination (as per the Eukaryotic Promoter Data Base EPDB) could also be used to drive expression of a transgene. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

One will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal and the bovine growth hormone polyadenylation signal, convenient and known to function well in various target cells. Also contemplated as an element of the expression cassette is a terminator. These elements can serve to enhance message levels and to minimize read through from the cassette into other sequences.

A specific initiation signal also may be required for efficient translation of coding sequences. These signals include the ATG initiation codon and adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements (Bittner et al., 1987).

In various embodiments of the invention, the expression construct may comprise a virus or engineered construct derived from a viral genome. The ability of certain viruses to enter cells via receptor-mediated endocytosis and to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986) and adeno-associated viruses. Retroviruses also are attractive gene transfer vehicles (Nicolas and Rubenstein, 1988; Temin, 1986) as are vaccinia virus (Ridgeway, 1988) and adeno-associated virus (Ridgeway, 1988). Such vectors may be used to (i) transform cell lines in vitro for the purpose of expressing proteins of interest or (ii) to transform cells in vitro or in vivo to provide therapeutic polypeptides in a gene therapy scenario.

B. Viral Vectors

Viral vectors are a kind of expression construct that utilizes viral sequences to introduce nucleic acid and possibly proteins into a cell. The ability of certain viruses to infect cells or enter cells via receptor-mediated endocytosis, and to integrate into host cell genome and express viral genes stably and efficiently have made them attractive candidates for the transfer of foreign nucleic acids into cells (e.g., mammalian cells). Vector components of the present invention may be a viral vector that encode one or more candidate substance or other components such as, for example, an immunomodulator or adjuvant for the candidate substance. Non-limiting examples of virus vectors that may be used to deliver a nucleic acid of the present invention are described below.

1. Adenoviral Vectors

### a. Virus Characteristics

Adenovirus is a non-enveloped double-stranded DNA virus. The virion consists of a DNA-protein core within a protein capsid. Virions bind to a specific cellular receptor, are endocytosed, and the genome is extruded from endosomes and transported to the nucleus. The genome is about 36 kB, encoding about 36 genes. In the nucleus, the “immediate early” E1A proteins are expressed initially, and these proteins induce expression of the “delayed early” proteins encoded by the E1B, E2, E3, and E4 transcription units. Virions assemble in the nucleus at about 1 day post infection (p.i.), and after 2-3 days the cell lyses and releases progeny virus. Cell lysis is mediated by the E3 11.6K protein, which has been renamed “adenovirus death protein” (ADP).

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP (located at 16.8 m.u.) is particularly efficient during the late phase of infection, and all the mRNA's issued from this promoter possess a 5′-tripartite leader (TPL) sequence which makes them preferred mRNA's for translation.

Adenovirus may be any of the 51 different known serotypes or subgroups A-F. Adenovirus type 5 of subgroup C is the human adenovirus about which the most biochemical and genetic information is known, and it has historically been used for most constructions employing adenovirus as a vector. Recombinant adenovirus often is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Viruses used in gene therapy may be either replication-competent or replication-deficient. Generation and propagation of the adenovirus vectors which are replication-deficient depends on a helper cell line, the prototype being 293 cells, prepared by transforming human embryonic kidney cells with Ad5 DNA fragments; this cell line constitutively expresses E1 proteins (Graham et al., 1977). However, helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Racher et al. (1995) have disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Adenovirus growth and manipulation is known to those of skill in the art, and exhibits broad host range in vitro and in vivo. This group of viruses can be obtained in high titers, e.g., 10⁹-10¹³ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1992). Animal studies have suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

### b. Engineering

As stated above, Ad vectors are based on recombinant Ad's that are either replication-defective or replication-competent. Typical replication-defective Ad vectors lack the E1A and E1B genes (collectively known as E1) and contain in their place an expression cassette consisting of a promoter and pre-mRNA processing signals which drive expression of a foreign gene. These vectors are unable to replicate because they lack the E1A genes required to induce Ad gene expression and DNA replication. In addition, the E3 genes can be deleted because they are not essential for virus replication in cultured cells. It is recognized in the art that replication-defective Ad vectors have several characteristics that make them suboptimal for use in therapy. For example, production of replication-defective vectors requires that they be grown on a complementing cell line that provides the E1A proteins in trans.

Several groups have also proposed using replication-competent Ad vectors for therapeutic use. Replication-competent vectors retain Ad genes essential for replication, and thus do not require complementing cell lines to replicate. Replication-competent Ad vectors lyse cells as a natural part of the life cycle of the vector. An advantage of replication-competent Ad vectors occurs when the vector is engineered to encode and express a foreign protein. Such vectors would be expected to greatly amplify synthesis of the encoded protein in vivo as the vector replicates. For use as anti-cancer agents, replication-competent viral vectors would theoretically be advantageous in that they would replicate and spread throughout the tumor, not just in the initially infected cells as is the case with replication-defective vectors.

Yet another approach is to create viruses that are conditionally-replication competent. Onyx Pharmaceuticals recently reported on adenovirus-based anti-cancer vectors which are replication-deficient in non-neoplastic cells, but which exhibit a replication phenotype in neoplastic cells lacking functional p53 and/or retinoblastoma (pRB) tumor suppressor proteins (U.S. Pat. No. 5,677,178). This phenotype is reportedly accomplished by using recombinant adenoviruses containing a mutation in the E1B region that renders the encoded E1B-55K protein incapable of binding to p53 and/or a mutation(s) in the E1A region which make the encoded E1A protein (p289R or p243R) incapable of binding to pRB and/or p300 and/or p107. E1B-55K has at least two independent functions: it binds and inactivates the tumor suppressor protein p53, and it is required for efficient transport of Ad mRNA from the nucleus. Because these E1B and E1A viral proteins are involved in forcing cells into S-phase, which is required for replication of adenovirus DNA, and because the p53 and pRB proteins block cell cycle progression, the recombinant adenovirus vectors described by Onyx should replicate in cells defective in p53 and/or pRB, which is the case for many cancer cells, but not in cells with wild-type p53 and/or pRB.

Another replication-competent adenovirus vector has the gene for E1B-55K replaced with the herpes simplex virus thymidine kinase gene (Wilder et al., 1999a). The group that constructed this vector reported that the combination of the vector plus gancyclovir showed a therapeutic effect on a human colon cancer in a nude mouse model (Wilder et al., 1999b). However, this vector lacks the gene for ADP, and accordingly, the vector will lyse cells and spread from cell-to-cell less efficiently than an equivalent vector that expresses ADP.

One may also take advantage of various promoter systems to create adenovirus vectors which overexpress p53. Vectors may also be replication competent or conditionally replicative. Other versions of engineered adenoviruses include disrupting E1A's ability to bind p300 and/or members of the Rb family members, or Ad vectors lacking expression of at least one E3 protein selected from the group consisting of 6.7K, gp19K, RIDα (also known as 10.4K); RIDβ (also known as 14.5K) and 14.7K. Because wild-type E3 proteins inhibit immune-mediated inflammation and/or apoptosis of Ad-infected cells, a recombinant adenovirus lacking one or more of these E3 proteins may stimulate infiltration of inflammatory and immune cells into a tumor treated with the adenovirus and that this host immune response will aid in destruction of the tumor as well as tumors that have metastasized. A mutation in the E3 region would impair its wild-type function, making the viral-infected cell susceptible to attack by the host's immune system. These viruses are described in detail in U.S. Pat. No. 6,627,190.

Other adenoviral vectors are described in U.S. Pat. Nos. 5,670,488; 5,747,869; 5,932,210; 5,981,225; 6,069,134; 6,136,594; 6,143,290; 6,210,939; 6,296,845; 6,410,010; and 6,511,184; U.S. Publication No. 2002/0028785.

### c. Oncolytic Vectors

Oncolytic viruses are also contemplated as vectors in the present invention. Oncolytic viruses are defined herein to generally refer to viruses that kill tumor or cancer cells more often than they kill normal cells. Exemplary oncolytic viruses include adenoviruses which overexpress ADP. These viruses are discussed in detail in U.S. Patent Application 20040213764, U.S. Patent Application 20020028785, and U.S. patent application Ser. No. 09/351,778, each of which is specifically incorporated by reference in its entirety into this section of the application and all other sections of the application. Exemplary oncolytic viruses are discussed elsewhere in this specification. One of ordinary skill in the art would be familiar with other oncolytic viruses that can be applied in the pharmaceutical compositions and methods of the present invention.

2. AAV Vectors

The nucleic acid may be introduced into the cell using adenovirus assisted transfection. Increased transfection efficiencies have been reported in cell systems using adenovirus coupled systems (Kelleher and Vos, 1994; Cotten et al., 1992; Curiel, 1994). Adeno-associated virus (AAV) is an attractive vector system for use in the methods of the present invention as it has a high frequency of integration and it can infect nondividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. Nos. 5,139,941 and 4,797,368, each incorporated herein by reference.

3. Retroviral Vectors

Retroviruses have promise as therapeutic vectors due to their ability to integrate their genes into the host genome, transferring a large amount of foreign genetic material, infecting a broad spectrum of species and cell types and of being packaged in special cell-lines (Miller, 1992).

In order to construct a retroviral vector, a nucleic acid is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136).

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and that is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

4. Herpesvirus Vectors

Herpes simplex virus (HSV) has generated considerable interest in treating nervous system disorders due to its tropism for neuronal cells, but this vector also can be exploited for other tissues given its wide host range. Another factor that makes HSV an attractive vector is the size and organization of the genome. Because HSV is large, incorporation of multiple genes or expression cassettes is less problematic than in other smaller viral systems. In addition, the availability of different viral control sequences with varying performance (temporal, strength, etc.) makes it possible to control expression to a greater extent than in other systems. It also is an advantage that the virus has relatively few spliced messages, further easing genetic manipulations.

HSV also is relatively easy to manipulate and can be grown to high titers. Thus, delivery is less of a problem, both in terms of volumes needed to attain sufficient MOI and in a lessened need for repeat dosings. For a review of HSV as a gene therapy vector, see Glorioso et al. (1995).

HSV, designated with subtypes 1 and 2, are enveloped viruses that are among the most common infectious agents encountered by humans, infecting millions of human subjects worldwide. The large, complex, double-stranded DNA genome encodes for dozens of different gene products, some of which derive from spliced transcripts. In addition to virion and envelope structural components, the virus encodes numerous other proteins including a protease, a ribonucleotides reductase, a DNA polymerase, a ssDNA binding protein, a helicase/primase, a DNA dependent ATPase, a dUTPase and others.

HSV genes form several groups whose expression is coordinately regulated and sequentially ordered in a cascade fashion (Honess and Roizman, 1974; Honess and Roizman 1975). The expression of α genes, the first set of genes to be expressed after infection, is enhanced by the virion protein number 16, or α-transinducing factor (Post et al., 1981; Batterson and Roizman, 1983). The expression of β genes requires functional α gene products, most notably ICP4, which is encoded by the α4 gene (DeLuca et al., 1985). γ genes, a heterogeneous group of genes encoding largely virion structural proteins, require the onset of viral DNA synthesis for optimal expression (Holland et al., 1980).

In line with the complexity of the genome, the life cycle of HSV is quite involved. In addition to the lytic cycle, which results in synthesis of virus particles and, eventually, cell death, the virus has the capability to enter a latent state in which the genome is maintained in neural ganglia until some as of yet undefined signal triggers a recurrence of the lytic cycle. Avirulent variants of HSV have been developed and are readily available for use in gene therapy contexts (U.S. Pat. No. 5,672,344).

5. Vaccinia Virus Vectors

Vaccinia virus vectors have been used extensively because of the ease of their construction, relatively high levels of expression obtained, wide host range and large capacity for carrying DNA. Vaccinia contains a linear, double-stranded DNA genome of about 186 kb that exhibits a marked “A-T” preference. Inverted terminal repeats of about 10.5 kb flank the genome. The majority of essential genes appear to map within the central region, which is most highly conserved among poxviruses. Estimated open reading frames in vaccinia virus number from 150 to 200. Although both strands are coding, extensive overlap of reading frames is not common.

At least 25 kb can be inserted into the vaccinia virus genome (Smith and Moss, 1983). Prototypical vaccinia vectors contain.transgenes inserted into the viral thymidine kinase gene via homologous recombination. Vectors are selected on the basis of a tk-phenotype. Inclusion of the untranslated leader sequence of encephalomyocarditis virus, the level of expression is higher than that of conventional vectors, with the transgenes accumulating at 10% or more of the infected cell's protein in 24 h (Elroy-Stein et al., 1989).

6. Delivery Using Modified Viruses

A nucleic acid to be delivered may be housed within an infective virus that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

C. Non-Viral Delivery

Lipid-based non-viral formulations provide an alternative to viral gene therapies. Although many cell culture studies have documented lipid-based non-viral gene transfer, systemic gene delivery via lipid-based formulations has been limited. A major limitation of non-viral lipid-based gene delivery is the toxicity of the cationic lipids that comprise the non-viral delivery vehicle. The in vivo toxicity of liposomes partially explains the discrepancy between in vitro and in vivo gene transfer results. Another factor contributing to this contradictory data is the difference in liposome stability in the presence and absence of serum proteins. The interaction between liposomes and serum proteins has a dramatic impact on the stability characteristics of liposomes (Yang and Huang, 1997). Cationic liposomes attract and bind negatively charged serum proteins. Liposomes coated by serum proteins are either dissolved or taken up by macrophages leading to their removal from circulation. Current in vivo liposomal delivery methods use aerosolization, subcutaneous, intradermal, intratumoral, or intracranial injection to avoid the toxicity and stability problems associated with cationic lipids in the circulation. The interaction of liposomes and plasma proteins is largely responsible for the disparity between the efficiency of in vitro (Felgner et al., 1987) and in vivo gene transfer (Zhu et al., 1993; Philip et al., 1993; Solodin et al., 1995; Liu et al., 1995; Thierry et al., 1995; Tsukamoto et al., 1995; Aksentijevich et al., 1996).

Recent advances in liposome formulations have improved the efficiency of gene transfer in vivo (Templeton et al. 1997; WO 98/07408, incorporated herein by reference). A novel liposomal formulation composed of an equimolar ratio of 1,2-bis(oleoyloxy)-3-(trimethyl ammonio)propane (DOTAP) and cholesterol significantly enhances systemic in vivo gene transfer, approximately 150-fold. The DOTAP:cholesterol lipid formulation is said to form a unique structure termed a “sandwich liposome.” This formulation is reported to “sandwich” DNA between an invaginated bilayer or “vase” structure. Beneficial characteristics of these liposomes include a positive to negative charge or p, colloidal stabilization by cholesterol, two-dimensional DNA packing and increased serum stability.

The production of lipid formulations often is accomplished by sonication or serial extrusion of liposomal mixtures after (I) reverse phase evaporation (II) dehydration-rehydration (III) detergent dialysis and (IV) thin film hydration. Once manufactured, lipid structures can be used to encapsulate compounds that are toxic (chemotherapeutics) or labile (nucleic acids) when in circulation. Liposomal encapsulation has resulted in a lower toxicity and a longer serum half-life for such compounds (Gabizon et al., 1990). Numerous disease treatments are using lipid based gene transfer strategies to enhance conventional or establish novel therapies, in particular therapies for treating hyperproliferative diseases.

Liposomes are vesicular structures characterized by a lipid bilayer and an inner aqueous medium. Multilamellar liposomes have multiple lipid layers separated by aqueous medium. They form spontaneously when lipids are suspended in an excess of aqueous solution. The lipid components undergo self-rearrangement before the formation of structures that entrap water and dissolved solutes between the lipid bilayers (Ghosh and Bachhawat, 1991). Lipophilic molecules or molecules with lipophilic regions may also dissolve in or associate with the lipid bilayer.

The liposomes are capable of carrying biologically active nucleic acids, such that the nucleic acids are completely sequestered. The liposome may contain one or more nucleic acids and is administered to a mammalian host to efficiently deliver its contents to a target cell. The liposomes may comprise DOTAP and cholesterol or a cholesterol derivative. In certain embodiments, the ratio of DOTAP to cholesterol, cholesterol derivative or cholesterol mixture is about 10:1 to about 1:10, about 9:1 to about 1:9, about 8:1 to about 1:8, about 7:1 to about 1:7, about 6:1 to about 1:6, about 5:1 to about 1:5, about 4:1 to about 1:4, about 3:1 to 1:3, 2:1 to 1:2, and 1:1. In further embodiments, the DOTAP and/or cholesterol concentrations are about 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, or 30 mM. The DOTAP and/or Cholesterol concentration can be between about 1 mM to about 20 mM, 1 mM to about 18 mM, 1 mM to about 16 mM, about 1 mM to about 14 mM, about 1 mM to about 12 mM, about 1 mM to about 10 mM, 1 to 8 mM, 2 to 7 mM, 3 to 6 mM and 4 to 5 mM. Cholesterol derivatives may be readily substituted for the cholesterol or mixed with the cholesterol in the present invention. Many cholesterol derivatives are known to the skilled artisan. Examples include but are not limited to cholesterol acetate and cholesterol oleate. A cholesterol mixture refers to a composition that contains at least one cholesterol or cholesterol derivative.

The formulation may also be extruded using a membrane or filter, and this may be performed multiple times. Such techniques are well-known to those of skill in the art, for example in Martin (1990). Extrusion may be performed to homogenize the formulation or limit its size. A contemplated method for preparing liposomes in certain embodiments is heating, sonicating, and sequential extrusion of the lipids through filters of decreasing pore size, thereby resulting in the formation of small, stable liposome structures. This preparation produces liposomal complexesor liposomes only of appropriate and uniform size, which are structurally stable and produce maximal activity.

For example, it is contemplated in certain embodiments of the present invention that DOTAP:Cholesterol liposomes are prepared by the methods of Templeton et al. (1997; incorporated herein by reference). Thus, in one embodiment, DOTAP (cationic lipid) is mixed with cholesterol (neutral lipid) at equimolar concentrations. This mixture of powdered lipids is then dissolved with chloroform, the solution dried to a thin film and the film hydrated in water containing 5% dextrose (w/v) to give a final concentration of 20 mM DOTAP and 20 mM cholesterol. The hydrated lipid film is rotated in a 50° C. water bath for 45 minutes, then at 35° C. for an additional 10 minutes and left standing at room temperature overnight. The following day the mixture is sonicated for 5 minutes at 50° C. The sonicated mixture is transferred to a tube and heated for 10 minutes at 50° C. This mixture is sequentially extruded through syringe filters of decreasing pore size (1 μm, 0.45 μm, 0.2 μm, 0.1 μm).

It also is contemplated that other liposome formulations and methods of preparation may be combined to impart desired DOTAP:Cholesterol liposome characteristics. Alternate methods of preparing lipid-based formulations for nucleic acid delivery are described by Saravolac et al. (WO 99/18933). Detailed are methods in which lipids compositions are formulated specifically to encapsulate nucleic acids. In another liposome formulation, an amphipathic vehicle called a solvent dilution microcarrier (SDMC) enables integration of particular molecules into the bi-layer of the lipid vehicle (U.S. Pat. No. 5,879,703). The SDMCs can be used to deliver lipopolysaccharides, polypeptides, nucleic acids and the like. Of course, any other methods of liposome preparation can be used by the skilled artisan to obtain a desired liposome formulation in the present invention.

Other formulations for delivering genes into tumors known to those skilled in the art may also be utilized in the invention. The present invention also includes nanoparticle liposome formulations for topical delivery of a nucleic acid expression construct. For instance, the liposome formulation may comprise DOTAP and cholesterol. An example of such a formulation containing a nucleic acid expression construct is shown below.

Cationic lipid (DOTAP) may be mixed with the neutral lipid cholesterol (Chol) at equimolar concentrations (Avanti Lipids). The mixed powdered lipids can be dissolved in HPLC-grade chloroform (Mallinckrodt, Chesterfield, Mo.) in a 1-L round-bottomed flask. After dissolution, the solution may be rotated on a Buchi rotary evaporator at 30° C. for 30 min to make a thin film. The flask containing the thin lipid film may then be dried under a vacuum for 15 min. Once drying is complete, the film may be hydrated in 5% dextrose in water (D5W) to give a final concentration of 20 mM DOTAP and 20 mM cholesterol, referred to as 20 mM DOTAP:Chol. The hydrated lipid film may be rotated in a water bath at 50° C. for 45 min and then at 35° C. for 10 min. The mixture may then be allowed to stand in the parafilm-covered flask at room temperature overnight, followed by sonication at low frequency (Lab-Line, TranSonic 820/H) for 5 min at 50° C. After sonication, the mixture may be transferred to a tube and heated for 10 min at 50° C., followed by sequential extrusion through Whatman (Kent, UK) filters of decreasing size: 1.0, 0.45, 0.2 and 0.1 μm using syringes. Whatman Anotop filters, 0.2 μm and 0.1 μm, may be used. Upon extrustion, the liposomes can be stored under argon gas at 4° C.

A nucleic acid expression construct in the form of plasmid DNA, for example 150 μg may be diluted in D5W. Stored liposomes may also be diluted in a separate solution of D5W. Equal volumes of both the DNA solution and the liposome solution can then be mixed to give a final concentration of, for example, 150 μg DNA/300 μl volume (2.5 μg/5 μl). Dilution and mixing may be performed at room temperature. The DNA solution mau then be added rapidly at the surface of the liposome solution by using a Pipetman pipet tip. The DNA:liposome mixture can then be mixed rapidly up and down twice in the pipette tip to form DOTAP:Cholesterol nucleic acid expression construct complexes.

Using the teachings of the specification and the knowledge of those skilled in the art, one can conduct tests to determine the particle size of the DOTAP:Chol-nucleic acid expression complex. For instance, the particle size of the DOTAP:Chol-nucleic acid expression construct complex may be determined using the N4-Coulter Particle Size analyzer (Beckman-Coulter). For this determination, 5 μl of the freshly prepared complex should be diluted in 1 ml of water prior to particle size determination. Additionally, a spectrophotometric reading of the complex at O.D. 400 nm may also be employed in analysis. For this analysis, 5 μl of the sample may be diluted in 95 μl of D5W to make a final volume of 100 μl. Applying the formulation techniques above with the size analysis methods should demonstrate a size of the complex between 374-400 nm.

Nanocapsules can generally entrap compounds in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use in the present invention, and such particles may be are easily made. Methods pertaining to the use of nanoparticles that may be used with the methods and compositions of the present invention include U.S. Pat. Nos. 6,555,376, 6,797,704, U.S. Patent Appn. 20050143336, U.S. Patent Appn. 20050196343 and U.S. Patent Appn. 20050260276, each of which is herein specifically incorporated by reference in its entirety. U.S. Patent Publication 20050143336 for example, provides examples of nanoparticle formulations containing tumor suppressor genes such as p53 and FUS-1 in nucleic acid form which are complexed with cationic lipids such as DOTAP or neutral lipids such as DOPE which form liposomes.

D. Vector Delivery and Cell Transformation

Suitable methods for nucleic acid delivery for transformation of an organelle, a cell, a tissue or an organism for use with the current invention are believed to include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by ex vivo transfection (Wilson et al., 1989; Nabel et al., 1989), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (WO 94/09699 and WO 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); and any combination of such methods.

E. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAxBAc® from INVITROGEN® and BACPACK™ BACULOVIRUS EXPRESSION SYSTEM FROM CLONTECH®.

Other examples of expression systems include STRATAGENE®'s COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

It is contemplated that the therapeutic gene may be “overexpressed,” i.e., expressed in increased levels relative to its natural expression in cells. Such overexpression may be assessed by a variety of methods, including radio-labeling and/or protein purification. However, simple and direct methods are preferred, for example, those involving SDS/PAGE and protein staining or western blotting, followed by quantitative analyses, such as densitometric scanning of the resultant gel or blot. A specific increase in the level of the recombinant protein, polypeptide or peptide in comparison to the level in natural cells is indicative of overexpression, as is a relative abundance of the specific protein, polypeptides or peptides in relation to the other proteins produced by the host cell, e.g., visible on a gel.

In some embodiments, the expressed proteinaceous sequence forms an inclusion body in the host cell, the host cells are lysed, for example, by disruption in a cell homogenizer, washed and/or centrifuged to separate the dense inclusion bodies and cell membranes from the soluble cell components. This centrifugation can be performed under conditions whereby the dense inclusion bodies are selectively enriched by incorporation of sugars, such as sucrose, into the buffer and centrifugation at a selective speed. Inclusion bodies may be solubilized in solutions containing high concentrations of urea (e.g., 8 M) or chaotropic agents such as guanidine hydrochloride in the presence of reducing agents, such as β-mercaptoethanol or DTT (dithiothreitol), and refolded into a more desirable conformation, as would be known to one of ordinary skill in the art.

The nucleotide and protein sequences for therapeutic genes have been previously disclosed, and may be found at computerized databases known to those of ordinary skill in the art. One such database is the National Center for Biotechnology Information's Genbank and GenPept databases (www.ncbi.nlm.nih.gov/). The coding regions for these known genes may be amplified and/or expressed using the techniques disclosed herein or by any technique that would be known to those of ordinary skill in the art. Additionally, peptide sequences may be synthesized by methods known to those of ordinary skill in the art, such as peptide synthesis using automated peptide synthesis machines, such as those available from Applied Biosystems (Foster City, Calif.).

IV. Therapeutic Combinations

In accordance with the present invention, additional therapies may be applied with further benefit to the patients. Such therapies include radiation, chemotherapy, surgery, cytokines, immunotherapy, toxins, drugs, dietary, or a secondary gene therapy. Examples are discussed below.

To kill cancer cells, slow their growth, or to achieve any of the clinical endpoints discussed above, one may contact the cancer cell or tumor with primary gene therapy in combination with a second anti-cancer therapy. These two modalities are provided in a combined amount effective to kill or inhibit proliferation of the cancer cell, or to achieve the desired clinical endpoint, including increasing patient survival. This process may involve contacting the cancer cell or tumor with both modalities at the same time. This may be achieved by contacting cancer cell or tumor with a single composition or pharmacological formulation that includes both agents, or by contacting the cancer cell or tumor with two distinct compositions or formulations, at the same time, wherein one composition includes the primary gene therapy, and the other includes the second therapy.

Alternatively, the primary gene therapy may precede or follow the second therapy by intervals ranging from minutes to weeks. In embodiments where the two modalities are applied separately to the cancer cell or tumor, one would generally ensure that a significant period of time did not expire between the time of each delivery, such that both would still be able to exert an advantageously combined effect on the cancer cell or tumor. In such instances, it is contemplated that one would contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6, or 7) to several weeks (1, 2, 3, 4, 5, 6, 7 or 8) lapse between the respective administrations.

It is also conceivable that more than one administration of each modality will be desired. Various combinations may be employed, where the primary gene therapy is “A” and the second therapy is “B”:

A/B/A B/A/B A/B/A A/A/B A/B/B B/A/A B/B/B/A B/A/B/B
B/B/A/B A/A/B/B A/B/A/B A/B/B/A B/B/A/A B/A/B/A
B/A/A/B A/A/A/B B/A/A/A A/B/A/A A/A/B/A A/B/B/B

The terms “contacted” and “exposed,” when applied to a cancer cell or tumor, are used herein to describe the process by which an agent or agents is/are delivered to a cancer cell or tumor or are placed in direct juxtaposition thereto.

A. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Intratumoral injection prior to surgery or upon excision of part of all of cancerous cells, tissue, or tumor, a cavity may be formed in the body. Treatment may be accomplished by perfusion, direct injection or local application of these areas with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

B. Secondary Gene Therapy

In another embodiment, the secondary treatment is a distinct gene therapy in which a second gene is administered to the subject. Delivery of a vector encoding the primary gene therapy in conjunction with a second vector encoding a distinct gene therapy product may be utilized. Alternatively, a single vector encoding both genes may be used. A variety of molecules are encompassed within this embodiment, and are discussed above. See “Gene Therapy: Treating Disease by Repairing Genes”, 2004); “Gene Therapy Protocols (Methods in Molecular Medicine)”, 2002).

C. Chemotherapy

A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

1. Alkylating agents

Alkylating agents are drugs that directly interact with genomic DNA to prevent the cancer cell from proliferating. This category of chemotherapeutic drugs represents agents that affect all phases of the cell cycle, that is, they are not phase-specific. Alkylating agents can be implemented to treat chronic leukemia, non-Hodgkin's lymphoma, Hodgkin's disease, multiple myeloma, and particular cancers of the breast, lung, and ovary. They include: busulfan, chlorambucil, cisplatin, cyclophosphamide (cytoxan), dacarbazine, ifosfamide, mechlorethamine (mustargen), and melphalan. Troglitazaone can be used to treat cancer in combination with any one or more of these alkylating agents, some of which are discussed below.

### a. Busulfan

Busulfan (also known as myleran) is a bifunctional alkylating agent. Busulfan is known chemically as 1,4-butanediol dimethanesulfonate.

Busulfan is not a structural analog of the nitrogen mustards. Busulfan is available in tablet form for oral administration. Each scored tablet contains 2 mg busulfan and the inactive ingredients magnesium stearate and sodium chloride.

Busulfan is indicated for the palliative treatment of chronic myelogenous (myeloid, myelocytic, granulocytic) leukemia. Although not curative, busulfan reduces the total granulocyte mass, relieves symptoms of the disease, and improves the clinical state of the patient. Approximately 90% of adults with previously untreated chronic myelogenous leukemia will obtain hematologic remission with regression or stabilization of organomegaly following the use of busulfan. It has been shown to be superior to splenic irradiation with respect to survival times and maintenance of hemoglobin levels, and to be equivalent to irradiation at controlling splenomegaly.

### b. Chlorambucil

Chlorambucil (also known as leukeran) is a bifunctional alkylating agent of the nitrogen mustard type that has been found active against selected human neoplastic diseases. Chlorambucil is known chemically as 4-[bis(2-chlorethyl)amino] benzenebutanoic acid.

Chlorambucil is available in tablet form for oral administration. It is rapidly and completely absorbed from the gastrointestinal tract. After single oral doses of 0.6-1.2 mg/kg, peak plasma chlorambucil levels are reached within one hour and the terminal half-life of the parent drug is estimated at 1.5 hours. 0.1 to 0.2 mg/kg/day or 3 to 6 mg/m²/day or alternatively 0.4 mg/kg may be used for antineoplastic treatment. Treatment regimes are well know to those of skill in the art and can be found in the “Physicians Desk Reference” and in “Remington's Pharmaceutical Sciences” referenced herein.

Chlorambucil is indicated in the treatment of chronic lymphatic (lymphocytic) leukemia, malignant lymphomas including lymphosarcoma, giant follicular lymphoma and Hodgkin's disease. It is not curative in any of these disorders but may produce clinically useful palliation. Thus, it can be used in combination with troglitazone in the treatment of cancer.

### c. Platinum-Containing Compounds

Platinum cytotoxics play an important role globally in the management of solid tumors. Cisplatin has been widely used to treat cancers such as metastatic testicular or ovarian carcinoma, advanced bladder cancer, head or neck cancer, cervical cancer, lung cancer or other tumors. Cisplatin can be used alone or in combination with other agents, with efficacious doses used in clinical applications of 15-20 mg/m² for 5 days every three weeks for a total of three courses. Exemplary doses may be 0.50 mg/m², 1.0 mg/m², 1.50 mg/m², 1.75 mg/m², 2.0 mg/m², 3.0 mg/m², 4.0 mg/m², 5.0 mg/m², 10 mg/m². Of course, all of these dosages are exemplary, and any dosage in-between these points is also expected to be of use in the invention. Cisplatin is not absorbed orally and must therefore be delivered via injection intravenously, subcutaneously, intratumorally or intraperitoneally.

Carboplatin, another platinum compound, is associated with neurotoxicity, but has become the leading product in the U.S. due largely to the ease with which toxicity profiles can be managed. Oxaliplatin (Europe) and nedaplatin (in Japan) have also been introduced. Platinum compounds can be used effectively in combination with 5-FU. CTI is testing two additional platinum compounds—BBR 3464 and BBR 3610—to identify appropriate clinical formulations.

### d. Cyclophosphamide

Cyclophosphamide is 2H-1,3,2-Oxazaphosphorin-2-amine, N,N-bis(2-chloroethyl)tetrahydro-, 2-oxide, monohydrate; termed Cytoxan available from Mead Johnson; and Neosar available from Adria. Cyclophosphamide is prepared by condensing 3-amino-1-propanol with N,N-bis(2-chlorethyl) phosphoramidic dichloride [(ClCH₂CH₂)₂N--POCl₂] in dioxane solution under the catalytic influence of triethylamine. The condensation is double, involving both the hydroxyl and the amino groups, thus effecting the cyclization.

Unlike other β-chloroethylamino alkylators, it does not cyclize readily to the active ethyleneimonium form until activated by hepatic enzymes. Thus, the substance is stable in the gastrointestinal tract, tolerated well and effective by the oral and parental routes and does not cause local vesication, necrosis, phlebitis or even pain.

Suitable doses for adults include, orally, 1 to 5 mg/kg/day (usually in combination), depending upon gastrointestinal tolerance; or 1 to 2 mg/kg/day; intravenously, initially 40 to 50 mg/kg in divided doses over a period of 2 to 5 days or 10 to 15 mg/kg every 7 to 10 days or 3 to 5 mg/kg twice a week or 1.5 to 3 mg/kg/day. A dose 250 mg/kg/day may be administered as an antineoplastic. Because of gastrointestinal adverse effects, the intravenous route is preferred for loading. During maintenance, a leukocyte count of 3000 to 4000/mm³ usually is desired. The drug also sometimes is administered intramuscularly, by infiltration or into body cavities. It is available in dosage forms for injection of 100, 200 and 500 mg, and tablets of 25 and 50 mg the skilled artisan is referred to “Remington's Pharmaceutical Sciences” 15th Edition, chapter 61, incorporate herein as a reference, for details on doses for administration.

### e. Melphalan

Melphalan, also known as alkeran, L-phenylalanine mustard, phenylalanine mustard, L-PAM, or L-sarcolysin, is a phenylalanine derivative of nitrogen mustard. Melphalan is a bifunctional alkylating agent which is active against selective human neoplastic diseases. It is known chemically as 4-[bis(2-chloroethyl)amino]-L-phenylalanine.

Melphalan is the active L-isomer of the compound and was first synthesized in 1953 by Bergel and Stock; the D-isomer, known as medphalan, is less active against certain animal tumors, and the dose needed to produce effects on chromosomes is larger than that required with the L-isomer. The racemic (DL-) form is known as merphalan or sarcolysin. Melphalan is insoluble in water and has a pKa₁ of ˜2.1. Melphalan is available in tablet form for oral administration and has been used to treat multiple myeloma.

Available evidence suggests that about one third to one half of the patients with multiple myeloma show a favorable response to oral administration of the drug.

Melphalan has been used in the treatment of epithelial ovarian carcinoma. One commonly employed regimen for the treatment of ovarian carcinoma has been to administer melphalan at a dose of 0.2 mg/kg daily for five days as a single course. Courses are repeated every four to five weeks depending upon hematologic tolerance (Smith and Rutledge, 1975; Young et al., 1978). Alternatively the dose of melphalan used could be as low as 0.05 mg/kg/day or as high as 3 mg/kg/day or any dose in between these doses or above these doses. Some variation in dosage will necessarily occur depending on the condition of the subject being treated. The person responsible for administration will, in any event, determine the appropriate dose for the individual subject.

2. Antimetabolites

Antimetabolites disrupt DNA and RNA synthesis. Unlike alkylating agents, they specifically influence the cell cycle during S phase. They have used to combat chronic leukemias in addition to tumors of breast, ovary and the gastrointestinal tract. Antimetabolites include 5-fluorouracil (5-FU), cytarabine (Ara-C), fludarabine, gemcitabine, and methotrexate.

5-Fluorouracil (5-FU) has the chemical name of 5-fluoro-2,4(1H,3H)-pyrimidinedione. Its mechanism of action is thought to be by blocking the methylation reaction of deoxyuridylic acid to thymidylic acid. Thus, 5-FU interferes with the syntheisis of deoxyribonucleic acid (DNA) and to a lesser extent inhibits the formation of ribonucleic acid (RNA). Since DNA and RNA are essential for cell division and proliferation, it is thought that the effect of 5-FU is to create a thymidine deficiency leading to cell death. Thus, the effect of 5-FU is found in cells that rapidly divide, a characteristic of metastatic cancers.

3. Antitumor Antibiotics

Antitumor antibiotics have both antimicrobial and cytotoxic activity. These drugs also interfere with DNA by chemically inhibiting enzymes and mitosis or altering cellular membranes. These agents are not phase specific so they work in all phases of the cell cycle. Thus, they are widely used for a variety of cancers. Examples of antitumor antibiotics include bleomycin, dactinomycin, daunorubicin, doxorubicin (Adriamycin), and idarubicin, some of which are discussed in more detail below. Widely used in clinical setting for the treatment of neoplasms these compounds are administered through bolus injections intravenously at doses ranging from 25-75 mg/m² at 21 day intervals for adriamycin, to 35-100 mg/m² for etoposide intravenously or orally.

### a. Doxorubicin

Doxorubicin hydrochloride, 5,12-Naphthacenedione, (8s-cis)-10-[(3-amino-2,3,6-trideoxy-a-L-lyxo-hexopyranosyl)oxy]-7,8,9,10-tetrahydro-6,8,11-trihydroxy-8-(hydroxyacetyl)-1-methoxy-hydrochloride (hydroxydaunorubicin hydrochloride, Adriamycin) is used in a wide antineoplastic spectrum. It binds to DNA and inhibits nucleic acid synthesis, inhibits mitosis and promotes chromosomal aberrations.

Administered alone, it is the drug of first choice for the treatment of thyroid adenoma and primary hepatocellular carcinoma. It is a component of 31 first-choice combinations for the treatment of ovarian, endometrial and breast tumors, bronchogenic oat-cell carcinoma, non-small cell lung carcinoma, gastric adenocarcinoma, retinoblastoma, neuroblastoma, mycosis fungoides, pancreatic carcinoma, prostatic carcinoma, bladder carcinoma, myeloma, diffuse histiocytic lymphoma, Wilms' tumor, Hodgkin's disease, adrenal tumors, osteogenic sarcoma soft tissue sarcoma, Ewing's sarcoma, rhabdomyosarcoma and acute lymphocytic leukemia. It is an alternative drug for the treatment of islet cell, cervical, testicular and adrenocortical cancers. It is also an immunosuppressant.

Doxorubicin is absorbed poorly and must be administered intravenously. The pharmacokinetics are multicompartmental. Distribution phases have half-lives of 12 minutes and 3.3 hr. The elimination half-life is about 30 hr. Forty to 50% is secreted into the bile. Most of the remainder is metabolized in the liver, partly to an active metabolite (doxorubicinol), but a few percent is excreted into the urine. In the presence of liver impairment, the dose should be reduced.

Appropriate doses are, intravenous, adult, 60 to 75 mg/m² at 21-day intervals or 25 to 30 mg/m² on each of 2 or 3 successive days repeated at 3- or 4-wk intervals or 20 mg/m² once a week. The lowest dose should be used in elderly patients, when there is prior bone-marrow depression caused by prior chemotherapy or neoplastic marrow invasion, or when the drug is combined with other myelopoietic suppressant drugs. The dose should be reduced by 50% if the serum bilirubin lies between 1.2 and 3 mg/dL and by 75% if above 3 mg/dL. The lifetime total dose should not exceed 550 mg/m² in patients with normal heart function and 400 mg/m² in persons having received mediastinal irradiation. Alternatively, 30 mg/m² on each of 3 consecutive days, repeated every 4 wk. Exemplary doses may be 10 mg/m², 20 mg/m², 30 mg/m², 50 mg/m², 100 Mg/m², 150 Mg/m², 175 mg/m², 200 mg/m², 225 mg/m², 250 mg/m², 275 mg/m², 300 mg/m², 350 mg/m², 400 mg/m², 425 mg/m², 450 mg/m², 475 mg/m², 500 mg/m². Of course, all of these dosages are exemplary, and any dosage in-between these points is also expected to be of use in the invention.

In the present invention the inventors have employed troglitazone as an exemplary chemotherapeutic agent to synergistically enhance the antineoplastic effects of the doxorubicin in the treatment of cancers. Those of skill in the art will be able to use the invention as exemplified potentiate the effects of doxorubicin in a range of different pre-cancer and cancers.

### b. Daunorubicin

Daunorubicin hydrochloride, 5,12-Naphthacenedione, (8S-cis)-8-acetyl-10-[(3-amino-2,3,6-trideoxy-a-L-lyxo-hexanopyranosyl)oxy]-7,8,9,10-tetrahydro-6,8,11-trihydroxy-10-methoxy-, hydrochloride; also termed cerubidine and available from Wyeth. Daunorubicin intercalates into DNA, blocks DAN-directed RNA polymerase and inhibits DNA synthesis. It can prevent cell division in doses that do not interfere with nucleic acid synthesis.

In combination with other drugs it is included in the first-choice chemotherapy of acute myelocytic leukemia in adults (for induction of remission), acute lymphocytic leukemia and the acute phase of chronic myelocytic leukemia. Oral absorption is poor, and it must be given intravenously. The half-life of distribution is 45 minutes and of elimination, about 19 hr. The half-life of its active metabolite, daunorubicinol, is about 27 hr. Daunorubicin is metabolized mostly in the liver and also secreted into the bile (ca 40%). Dosage must be reduced in liver or renal insufficiencies.

Suitable doses are (base equivalent), intravenous adult, younger than 60 yr. 45 mg/m²/day (30 mg/m² for patients older than 60 yr) for 1, 2 or 3 days every 3 or 4 wk or 0.8 mg/kg/day for 3 to 6 days every 3 or 4 wk; no more than 550 mg/m² should be given in a lifetime, except only 450 mg/m² if there has been chest irradiation; children, 25 mg/m² once a week unless the age is less than 2 yr or the body surface less than 0.5 m, in which case the weight-based adult schedule is used. It is available in injectable dosage forms (base equivalent) 20 mg (as the base equivalent to 21.4 mg of the hydrochloride). Exemplary doses may be 10 mg/m², 20 mg/m², 30 mg/m², 50 mg/m², 100 Mg/m², 150 Mg/m², 175 mg/m², 200 mg/m², 225 mg/m², 250 mg/m², 275 mg/m², 300 mg/m², 350 mg/m², 400 mg/m², 425 mg/m², 450 mg/m², 475 mg/m², 500 mg/m². Of course, all of these dosages are exemplary, and any dosage in-between these points is also expected to be of use in the invention.

### c. Mitomycin

Mitomycin (also known as mutamycin and/or mitomycin-C) is an antibiotic isolated from the broth of Streptomyces caespitosus which has been shown to have antitumor activity. The compound is heat stable, has a high melting point, and is freely soluble in organic solvents.

Mitomycin selectively inhibits the synthesis of deoxyribonucleic acid (DNA). The guanine and cytosine content correlates with the degree of mitomycin-induced cross-linking. At high concentrations of the drug, cellular RNA and protein synthesis are also suppressed.

In humans, mitomycin is rapidly cleared from the serum after intravenous administration. Time required to reduce the serum concentration by 50% after a 30 mg. bolus injection is 17 minutes. After injection of 30 mg, 20 mg, or 10 mg I.V., the maximal serum concentrations were 2.4 mg/ml, 1.7 mg/ml, and 0.52 mg/ml, respectively. Clearance is effected primarily by metabolism in the liver, but metabolism occurs in other tissues as well. The rate of clearance is inversely proportional to the maximal serum concentration because, it is thought, of saturation of the degradative pathways. Approximately 10% of a dose of mitomycin is excreted unchanged in the urine. Since metabolic pathways are saturated at relatively low doses, the percent of a dose excreted in urine increases with increasing dose. In children, excretion of intravenously administered mitomycin is similar.

### d. Actinomycin D

Actinomycin D (Dactinomycin) [50-76-0]; C₆₂H₈₆N₁₂O₁₆ (1255.43) is an antineoplastic drug that inhibits DNA-dependent RNA polymerase. It is a component of first-choice combinations for treatment of choriocarcinoma, embryonal rhabdomyosarcoma, testicular tumor and Wilms' tumor. Tumors that fail to respond to systemic treatment sometimes respond to local perfusion. Dactinomycin potentiates radiotherapy. It is a secondary (efferent) immunosuppressive.

Actinomycin D is used in combination with primary surgery, radiotherapy, and other drugs, particularly vincristine and cyclophosphamide. Antineoplastic activity has also been noted in Ewing's tumor, Kaposi's sarcoma, and soft-tissue sarcomas. Dactinomycin can be effective in women with advanced cases of choriocarcinoma. It also produces consistent responses in combination with chlorambucil and methotrexate in patients with metastatic testicular carcinomas. A response may sometimes be observed in patients with Hodgkin's disease and non-Hodgkin's lymphomas. Dactinomycin has also been used to inhibit immunological responses, particularly the rejection of renal transplants.

Half of the dose is excreted intact into the bile and 10% into the urine; the half-life is about 36 hr. The drug does not pass the blood-brain barrier. Actinomycin D is supplied as a lyophilized powder (0/5 mg in each vial). The usual daily dose is 10 to 15 mg/kg; this is given intravenously for 5 days; if no manifestations of toxicity are encountered, additional courses may be given at intervals of 3 to 4 weeks. Daily injections of 100 to 400 mg have been given to children for 10 to 14 days; in other regimens, 3 to 6 mg/kg, for a total of 125 mg/kg, and weekly maintenance doses of 7.5 mg/kg have been used. Although it is safer to administer the drug into the tubing of an intravenous infusion, direct intravenous injections have been given, with the precaution of discarding the needle used to withdraw the drug from the vial in order to avoid subcutaneous reaction. Exemplary doses may be 100 mg/m², 150 mg/m², 175 mg/m², 200 mg/m², 225 mg/m², 250 mg/m², 275 mg/m², 300 mg/m², 350 mg/m², 400 mg/m², 425 mg/m², 450 mg/m², 475 mg/m², 500 mg/m². Of course, all of these dosages are exemplary, and any dosage in-between these points is also expected to be of use in the invention.

### e. Bleomycin

Bleomycin is a mixture of cytotoxic glycopeptide antibiotics isolated from a strain of Streptomyces verticillus. Although the exact mechanism of action of bleomycin is unknown, available evidence would seem to indicate that the main mode of action is the inhibition of DNA synthesis with some evidence of lesser inhibition of RNA and protein synthesis.

In mice, high concentrations of bleomycin are found in the skin, lungs, kidneys, peritoneum, and lymphatics. Tumor cells of the skin and lungs have been found to have high concentrations of bleomycin in contrast to the low concentrations found in hematopoietic tissue. The low concentrations of bleomycin found in bone marrow may be related to high levels of bleomycin degradative enzymes found in that tissue.

In patients with a creatinine clearance of >35 mL per minute, the serum or plasma terminal elimination half-life of bleomycin is approximately 115 minutes. In patients with a creatinine clearance of <35 mL per minute, the plasma or serum terminal elimination half-life increases exponentially as the creatinine clearance decreases. In humans, 60% to 70% of an administered dose is recovered in the urine as active bleomycin. Bleomycin may be given by the intramuscular, intravenous, or subcutaneous routes. It is freely soluble in water.

Bleomycin should be considered a palliative treatment. It has been shown to be useful in the management of the following neoplasms either as a single agent or in proven combinations with other approved chemotherapeutic agents in squamous cell carcinoma such as head and neck (including mouth, tongue, tonsil, nasopharynx, oropharynx, sinus, palate, lip, buccal mucosa, gingiva, epiglottis, larynx), skin, penis, cervix, and vulva. It has also been used in the treatment of lymphomas and testicular carcinoma.

Because of the possibility of an anaphylactoid reaction, lymphoma patients should be treated with two units or less for the first two doses. If no acute reaction occurs, then the regular dosage schedule may be followed.

Improvement of Hodgkin's Disease and testicular tumors is prompt and noted within 2 weeks. If no improvement is seen by this time, improvement is unlikely. Squamous cell cancers respond more slowly, sometimes requiring as long as 3 weeks before any improvement is noted.

4. Mitotic Inhibitors

Mitotic inhibitors include plant alkaloids and other natural agents that can inhibit either protein synthesis required for cell division or mitosis. They operate during a specific phase during the cell cycle. Mitotic inhibitors comprise docetaxel, etoposide (VP16), paclitaxel, taxol, taxotere, vinblastine, vincristine, and vinorelbine.

### a. Etoposide (VP16)

VP16 is also known as etoposide and is used primarily for treatment of testicular tumors, in combination with bleomycin and cisplatin, and in combination with cisplatin for small-cell carcinoma of the lung. It is also active against non-Hodgkin's lymphomas, acute nonlymphocytic leukemia, carcinoma of the breast, and Kaposi's sarcoma associated with acquired immunodeficiency syndrome (AIDS).

VP16 is available as a solution (20 mg/ml) for intravenous administration and as 50-mg, liquid-filled capsules for oral use. For small-cell carcinoma of the lung, the intravenous dose (in combination therapy) is can be as much as 100 mg/m² or as little as 2 mg/ m², routinely 35 mg/m², daily for 4 days, to 50 mg/m², daily for 5 days have also been used. When given orally, the dose should be doubled. Hence the doses for small cell lung carcinoma may be as high as 200-250 mg/m². The intravenous dose for testicular cancer (in combination therapy) is 50 to 100 mg/m² daily for 5 days, or 100 mg/m² on alternate days, for three doses. Cycles of therapy are usually repeated every 3 to 4 weeks. The drug should be administered slowly during a 30- to 60-minute infusion in order to avoid hypotension and bronchospasm, which are probably due to the solvents used in the formulation.

### b. Taxanes

Taxanes are a group of drugs that includes paclitaxel (Taxol) and docetaxel (Taxotere). Taxanes prevent growth of cancer cells by inhibiting the breakdown of microtubules, which normally occurs once a cell stops dividing. Thus, treated cells become so clogged with microtubules that they cannot grow and divide.

Paclitaxel (Taxol) is isolated from the bark of the ash tree, Taxus brevifolia. It binds to tubulin (at a site distinct from that used by the vinca alkaloids) and promotes the assembly of microtubules. It has activity against malignant melanoma and carcinoma of the ovary. Maximal doses are 30 mg/m² per day for 5 days or 210 to 250 mg/m² given once every 3 weeks. Of course, all of these dosages are exemplary, and any dosage in-between these points is also expected to be of use in the invention. Docetaxel, a compound that is similar to paclitaxel, and is also used to treat cancer. Docetaxel comes from the needles of the yew tree. The FDA has approved docetaxel to treat advanced breast, lung, and ovarian cancer.

### c. Vinblastine

Vinblastine is another example of a plant aklyloid that can be used in combination with troglitazone for the treatment of cancer and precancer. When cells are incubated with vinblastine, dissolution of the microtubules occurs.

Unpredictable absorption has been reported after oral administration of vinblastine or vincristine. At the usual clinical doses the peak concentration of each drug in plasma is approximately 0.4 mM. Vinblastine and vincristine bind to plasma proteins. They are extensively concentrated in platelets and to a lesser extent in leukocytes and erythrocytes.

After intravenous injection, vinblastine has a multiphasic pattern of clearance from the plasma; after distribution, drug disappears from plasma with half-lives of approximately 1 and 20 hours. Vinblastine is metabolized in the liver to biologically activate derivative desacetylvinblastine. Approximately 15% of an administered dose is detected intact in the urine, and about 10% is recovered in the feces after biliary excretion. Doses should be reduced in patients with hepatic dysfunction. At least a 50% reduction in dosage is indicated if the concentration of bilirubin in plasma is greater than 3 mg/dl (about 50 mM).

Vinblastine sulfate is available in preparations for injection. The drug is given intravenously; special precautions must be taken against subcutaneous extravasation, since this may cause painful irritation and ulceration. The drug should not be injected into an extremity with impaired circulation. After a single dose of 0.3 mg/kg of body weight, myelosuppression reaches its maximum in 7 to 10 days. If a moderate level of leukopenia (approximately 3000 cells/mm³) is not attained, the weekly dose may be increased gradually by increments of 0.05 mg/kg of body weight. In regimens designed to cure testicular cancer, vinblastine is used in doses of 0.3 mg/kg every 3 weeks irrespective of blood cell counts or toxicity.

The most important clinical use of vinblastine is with bleomycin and cisplatin in the curative therapy of metastatic testicular tumors. Beneficial responses have been reported in various lymphomas, particularly Hodgkin's disease, where significant improvement may be noted in 50 to 90% of cases. The effectiveness of vinblastine in a high proportion of lymphomas is not diminished when the disease is refractory to alkylating agents. It is also active in Kaposi's sarcoma, neuroblastoma, and Letterer-Siwe disease (histiocytosis X), as well as in carcinoma of the breast and choriocarcinoma in women.

Doses of vinblastine will be determined by the clinician according to the individual patients need. 0.1 to 0.3 mg/kg can be administered or 1.5 to 2 mg/m² can also be administered. Alternatively, 0.1 mg/m², 0.12 mg/m², 0.14 mg/m², 0.15 mg/m², 0.2 mg/m², 0.25 mg/m², 0.5 mg/m², 1.0 mg/m², 1.2 mg/m², 1.4 mg/m², 1.5 mg/m², 2.0 mg/m², 2.5 mg/m², 5.0 mg/m², 6 mg/m², 8 mg/m², 9 mg/m², 10 mg/m², 20 mg/m², can be given. Of course, all of these dosages are exemplary, and any dosage in-between these points is also expected to be of use in the invention.

### d. Vincristine

Vincristine blocks mitosis and produces metaphase arrest. It seems likely that most of the biological activities of this drug can be explained by its ability to bind specifically to tubulin and to block the ability of protein to polymerize into microtubules. Through disruption of the microtubules of the mitotic apparatus, cell division is arrested in metaphase. The inability to segregate chromosomes correctly during mitosis presumably leads to cell death.

The relatively low toxicity of vincristine for normal marrow cells and epithelial cells make this agent unusual among anti-neoplastic drugs, and it is often included in combination with other myelosuppressive agents.

Unpredictable absorption has been reported after oral administration of vinblastine or vincristine. At the usual clinical doses the peak concentration of each drug in plasma is approximately 0.4 mM.

Vinblastine and vincristine bind to plasma proteins. They are extensively concentrated in platelets and to a lesser extent in leukocytes and erythrocytes.

Vincristine has a multiphasic pattern of clearance from the plasma; the terminal half-life is about 24 hours. The drug is metabolized in the liver, but no biologically active derivatives have been identified. Doses should be reduced in patients with hepatic dysfunction. At least a 50% reduction in dosage is indicated if the concentration of bilirubin in plasma is greater than 3 mg/dl (about 50 mM).

Vincristine sulfate is available as a solution (1 mg/ml) for intravenous injection. Vincristine used together with corticosteroids is presently the treatment of choice to induce remissions in childhood leukemia; the optimal dosages for these drugs appear to be vincristine, intravenously, 2 mg/m² of body-surface area, weekly, and prednisone, orally, 40 mg/m², daily. Adult patients with Hodgkin's disease or non-Hodgkin's lymphomas usually receive vincristine as a part of a complex protocol. When used in the MOPP regimen, the recommended dose of vincristine is 1.4 mg/m². High doses of vincristine seem to be tolerated better by children with leukemia than by adults, who may experience sever neurological toxicity. Administration of the drug more frequently than every 7 days or at higher doses seems to increase the toxic manifestations without proportional improvement in the response rate. Precautions should also be used to avoid extravasation during intravenous administration of vincristine. Vincristine (and vinblastine) can be infused into the arterial blood supply of tumors in doses several times larger than those that can be administered intravenously with comparable toxicity.

Vincristine has been effective in Hodgkin's disease and other lymphomas. Although it appears to be somewhat less beneficial than vinblastine when used alone in Hodgkin's disease, when used with mechlorethamine, prednisone, and procarbazine (the so-called MOPP regimen), it is the preferred treatment for the advanced stages (III and IV) of this disease. In non-Hodgkin's lymphomas, vincristine is an important agent, particularly when used with cyclophosphamide, bleomycin, doxorubicin, and prednisone. Vincristine is more useful than vinblastine in lymphocytic leukemia. Beneficial response have been reported in patients with a variety of other neoplasms, particularly Wilms' tumor, neuroblastoma, brain tumors, rhabdomyosarcoma, and carcinomas of the breast, bladder, and the male and female reproductive systems.

Doses of vincristine for use will be determined by the clinician according to the individual patients need. 0.01 to 0.03 mg/kg or 0.4 to 1.4 mg/m2 can be administered or 1.5 to 2 mg/m² can also be administered. Alternatively 0.02 mg/m², 0.05 mg/m², 0.06 mg/m², 0.07 mg/m², 0.08 mg/m², 0.1 mg/m², 0.12 mg/m², 0.14 mg/m², 0.15 mg/m², 0.2 mg/m², 0.25 mg/m² can be given as a constant intravenous infusion. Of course, all of these dosages are exemplary, and any dosage in-between these points is also expected to be of use in the invention.

### e. Camptothecin

Camptothecin is an alkaloid derived from the chinese tree Camptotheca acuminata Decne. Camptothecin and its derivatives are unique in their ability to inhibit DNA Topoisomerase by stabilizing a covalent reaction intermediate, termed “the cleavable complex,” which ultimately causes tumor cell death. It is widely believed that camptothecin analogs exhibited remarkable anti-tumour and anti-leukaemia activity. Application of camptothecin in clinic is limited due to serious side effects and poor water-solubility. At present, some camptothecin analogs (topotecan; irinotecan), either synthetic or semi-synthetic, have been applied to cancer therapy and have shown satisfactory clinical effects. The molecular formula for camptothecin is C₂₀H₁₆N₂O₄, with a molecular weight of 348.36. It is provided as a yellow powder, and may be solubilized to a clear yellow solution at 50 mg/ml in DMSO 1N sodium hydroxide. It is stable for at least two years if stored at 2-8° C. in a dry, airtight, light-resistant environment.

5. Nitrosureas

Nitrosureas, like alkylating agents, inhibit DNA repair proteins. They are used to treat non-Hodgkin's lymphomas, multiple myeloma, malignant melanoma, in addition to brain tumors. Examples include carmustine and lomustine.

### a. Carmustine

Carmustine (sterile carmustine) is one of the nitrosoureas used in the treatment of certain neoplastic diseases. It is 1,3bis-(2-chloroethyl)-1-nitrosourea. It is lyophilized pale yellow flakes or congealed mass with a molecular weight of 214.06. It is highly soluble in alcohol and lipids, and poorly soluble in water. Carmustine is administered by intravenous infusion after reconstitution as recommended. Sterile carmustine is commonly available in 100 mg single dose vials of lyophilized material.

Although it is generally agreed that carmustine alkylates DNA and RNA, it is not cross resistant with other alkylators. As with other nitrosoureas, it may also inhibit several key enzymatic processes by carbamoylation of amino acids in proteins.

Carmustine is indicated as palliative therapy as a single agent or in established combination therapy with other approved chemotherapeutic agents in brain tumors such as glioblastoma, brainstem glioma, medullobladyoma, astrocytoma, ependymoma, and metastatic brain tumors. Also it has been used in combination with prednisone to treat multiple myeloma. Carmustine has proved useful, in the treatment of Hodgkin's Disease and in non-Hodgkin's lymphomas, as secondary therapy in combination with other approved drugs in patients who relapse while being treated with primary therapy, or who fail to respond to primary therapy.

The recommended dose of carmustine as a single agent in previously untreated patients is 150 to 200 mg/m² intravenously every 6 weeks. This may be given as a single dose or divided into daily injections such as 75 to 100 mg/m² on 2 successive days. When carmustine is used in combination with other myelosuppressive drugs or in patients in whom bone marrow reserve is depleted, the doses should be adjusted accordingly. Doses subsequent to the initial dose should be adjusted according to the hematologic response of the patient to the preceding dose. It is of course understood that other doses may be used in the present invention for example 10 mg/m², 20 mg/m², 30 mg/m², 40 mg/m², 50 mg/m², 60 mg/m², 70 mg/m², 80 mg/m², 90 mg/m² or 100 mg/m² . The skilled artisan is directed to “Remington's Pharmaceutical Sciences,” 15th Edition, chapter 61. Some variation in dosage will necessarily occur depending on the condition of the subject being treated. The person responsible for administration will, in any event, determine the appropriate dose for the individual subject.

### b. Lomustine

Lomustine is one of the nitrosoureas used in the treatment of certain neoplastic diseases. It is 1-(2-chloro-ethyl)-3-cyclohexyl-1 nitrosourea. It is a yellow powder with the empirical formula of C₉H₁₆ClN₃O₂ and a molecular weight of 233.71. Lomustine is soluble in 10% ethanol (0.05 mg per ml) and in absolute alcohol (70 mg per ml). Lomustine is relatively insoluble in water (<0.05 mg per ml). It is relatively unionized at a physiological pH. Inactive ingredients in lomustine capsules are magnesium stearate and mannitol.

Although it is generally agreed that lomustine alkylates DNA and RNA, it is not cross resistant with other alkylators. As with other nitrosoureas, it may also inhibit several key enzymatic processes by carbamoylation of amino acids in proteins.

Lomustine may be given orally. Following oral administration of radioactive lomustine at doses ranging from 30 mg/m² to 100 mg/m², about half of the radioactivity given was excreted in the form of degradation products within 24 hours. The serum half-life of the metabolites ranges from 16 hrs to 2 days. Tissue levels are comparable to plasma levels at 15 minutes after intravenous administration.

Lomustine has been shown to be useful as a single agent in addition to other treatment modalities, or in established combination therapy with other approved chemotherapeutic agents in both primary and metastatic brain tumors, in patients who have already received appropriate surgical and/or radiotherapeutic procedures. It has also proved effective in secondary therapy against Hodgkin's Disease in combination with other approved drugs in patients who relapse while being treated with primary therapy, or who fail to respond to primary therapy.

The recommended dose of lomustine in adults and children as a single agent in previously untreated patients is 130 mg/m² as a single oral dose every 6 weeks. In individuals with compromised bone marrow function, the dose should be reduced to 100 mg/m² every 6 weeks. When lomustine is used in combination with other myelosuppressive drugs, the doses should be adjusted accordingly. It is understood that other doses may be used for example, 20 mg/m², 30 mg/m², 40 mg/m², 50 mg/m², 60 mg/m², 70 mg/m², 80 mg/m², 90 mg/m², 100 mg/m², 120 mg/m² or any doses between these figures as determined by the clinician to be necessary for the individual being treated.

6. Other Agents

Other agents that may be used include bevacizumab (brand name Avastin®), gefitinib (Iressa®), trastuzumab (Herceptin®), cetuximab (Erbitux®), panitumumab (Vectibix®), bortezomib (Velcade®), and Gleevec. In addition, growth factor inhibitors and small molecule kinase inhibitors have utility in the present invention as well. All therapies described in Cancer: Principles and Practice of Oncology (7^{th} Ed.), 2004, and Clinical Oncology (3^{rd} Ed., 2004) are hereby incorporated by reference. The following additional therapies are encompassed, as well.

### a. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy, thus, could be used as part of a combined therapy, in conjunction with p53 gene therapy. The general approach for combined therapy is discussed below. Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. In addition, p53 itself may be an immunotherapy target. See U.S. Publication 2005/0171045, incorporated herein by reference.

Tumor Necrosis Factor is a glycoprotein that kills some kinds of cancer cells, activates cytokine production, activates macrophages and endothelial cells, promotes the production of collagen and collagenases, is an inflammatory mediator and also a mediator of septic shock, and promotes catabolism, fever and sleep. Some infectious agents cause tumor regression through the stimulation of TNF production. TNF can be quite toxic when used alone in effective doses, so that the optimal regimens probably will use it in lower doses in combination with other drugs. Its immunosuppressive actions are potentiated by gamma-interferon, so that the combination potentially is dangerous. A hybrid of TNF and interferon-α also has been found to possess anti-cancer activity.

### b. Hormonal Therapy

The use of sex hormones according to the methods described herein in the treatment of cancer. While the methods described herein are not limited to the treatment of a specific cancer, this use of hormones has benefits with respect to cancers of the breast, prostate, and endometrial (lining of the uterus). Examples of these hormones are estrogens, anti-estrogens, progesterones, and androgens.

Corticosteroid hormones are useful in treating some types of cancer (lymphoma, leukemias, and multiple myeloma). Corticosteroid hormones can increase the effectiveness of other chemotherapy agents, and consequently, they are frequently used in combination treatments. Prednisone and dexamethasone are examples of corticosteroid hormones.

D. Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly. Radiotherapy may be used to treat localized solid tumors, such as cancers of the skin, tongue, larynx, brain, breast, or cervix. It can also be used to treat leukemia and lymphoma (cancers of the blood-forming cells and lymphatic system, respectively).

Radiation therapy used according to the present invention may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer. This ensures that a higher radiation dose is given to the tumor. Healthy surrounding cells and nearby structures receive a lower dose of radiation, so the possibility of side effects is reduced. A device called a multi-leaf collimator has been developed and can be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of your internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques. Stereotactic radiotherapy is used to treat brain tumors. This technique directs the radiotherapy from many different angles so that the dose going to the tumour is very high and the dose affecting surrounding healthy tissue is very low. Before treatment, several scans are analysed by computers to ensure that the radiotherapy is precisely targeted, and the patient's head is held still in a specially made frame while receiving radiotherapy. Several doses are given.

Stereotactic radio-surgery (gamma knife) for brain and other tumors does not use a knife, but very precisely targeted beams of gamma radiotherapy from hundreds of different angles. Only one session of radiotherapy, taking about four to five hours, is needed. For this treatment you will have a specially made metal frame attached to your head. Then several scans and x-rays are carried out to find the precise area where the treatment is needed. During the radiotherapy for brain tumors, the patient lies with their head in a large helmet, which has hundreds of holes in it to allow the radiotherapy beams through. Related approaches permit positioning for the treatment of tumors in other areas of the body.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.

V. Pharmaceutical Compositions

According to the present invention, therapeutic compositions are administered to a subject. The phrases “pharmaceutically” or “pharmacologically acceptable” refer to compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the compositions, vectors or cells of the present invention, its use in therapeutic compositions is contemplated. Supplementary active ingredients also can be incorporated into the compositions.

In various embodiments, agents that might be delivered may be formulated and administered in any pharmacologically acceptable vehicle, such as parenteral, topical (e.g., applied to the skin, in a mouthwash), aerosal, liposomal, nasal or ophthalmic preparations. In certain embodiments, formulations may be designed for oral, inhalant or topical administration. In those situations, it would be clear to one of ordinary skill in the art the types of diluents that would be proper for the proposed use of the polypeptides and any secondary agents required.

Administration of compositions according to the present invention will be via any common route so long as the target tissue or surface is available via that route. This includes oral, nasal, buccal, respiratory, rectal, vaginal or topical. Alternatively, administration may be by intratumoral, intralesional, into tumor vasculature, local to a tumor, regional to a tumor, intradermal, subcutaneous, intramuscular, intraperitoneal or intravenous injection (systemic). Such compositions would normally be administered as pharmaceutically acceptable compositions, described supra.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. Routes of administration may be selected from intravenous, intrarterial, intrabuccal, intraperitoneal, intramuscular, subcutaneous, oral, topical, rectal, vaginal, nasal and intraocular.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

In a particular embodiment, liposomal formulations are contemplated. Liposomal encapsulation of pharmaceutical agents prolongs their half-lives when compared to conventional drug delivery systems. Because larger quantities can be protectively packaged, this allows the opportunity for dose-intensity of agents so delivered to cells.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Patients and Methods

Patient eligibility. The two studies were conducted concurrently with identical entry criteria apart from the administered dose of p53 gene therapy (ADVEXIN, Introgen Therapeutics Inc., Houston Tex.). Eligible patients had histologically-confirmed SCCHN, with cytologically-confirmed recurrence, excluding endolaryngeal recurrence, after first-line therapy administered with a curative intent (at least 50 Gy radiotherapy and/or surgery with or without chemotherapy). All lesions in the head and neck region were to be accessible to intratumoral treatment, or if not, any inaccessible lesions had to be separately evaluable and unlikely to impair the patient's ability to complete the study. At least one lesion had to be bidimensionally measurable (≧1 cm×1 cm by physical examination or ≧1 cm×2 cm by CT-scan or MRI). The total area of all bidimensionally measurable lesions had to be ≦30 cm², and the sum of the longest diameter of each bidimensionally and unidimensionally measurable lesion had to be ≧10 cm. In addition, patients were to be at least 18 years of age; be using barrier contraception; have a Kamofsky performance status (KPS) ≧60%, a life expectancy >12 weeks, adequate bone marrow and hepatic function (absolute neutrophil count [ANC]≧2×10^{9/}; platelet count≧100×10⁹/; total bilirubin≦upper limit of normal institutional range [ULN]; aspartate aminotransferase [AST] and alanine aminotransferase [ALT]≦1.5 ×ULN; alkaline phosphatase [AP]≦5 ULN); negative serology for HIV-1 and 2, hepatitis B surface antigen and hepatitis C. Patients were not eligible if they had central nervous system metastasis, prior radiation therapy to areas of measurable disease within 4 weeks of study entry (unless there was documented evidence of disease progression), systemic anti-cancer therapy within 4 weeks of study entry (6 weeks for nitrosoureas and mitomycin C), prior gene therapy using adenoviral vectors; prior autologous or allogeneic organ or tissue transplantation; serious concomitant medical conditions; a history of other malignancy unless curatively treated and disease free for ≧2 years; had participated in clinical studies of experimental agents within 4 weeks of study entry or were pregnant or lactating women.

The study protocols were reviewed by the National Institutes of Heath Recombinant DNA Advisory Committee. The protocol and all modifications were also approved by the applicable local or national ethics committees, biosafety committees, and regulatory authorities. Patients provided signed informed consent to participate in the study.

Study treatments. In the high-dose study (T201), patients were randomized to receive intratumoral injections of Ad-p53 either on days 1, 2 and 3 (3-day arm in high-dose) or days 1, 3, 5, 8, 10 and 12 (6-day arm) of each 28-day cycle. All patients in the low-dose study (T202) were treated according to the 3-day schedule. The dose per day ranged from, 5×10¹¹ to 2.5×10¹² viral particles (vp)/injection in the high-dose study and from 1-4×10⁹ vp/injection in the low-dose study, depending on the summarized lesion diameter. Study treatment was diluted in phosphate-buffered solution to a concentration of 4×10¹⁰ pfu/mL (high-dose study) or 1×10⁹ pfu/mL (low-dose study), and 0.1 or 0.2 mL was to be injected with a fine needle over the lesion volume at 1 cm intervals, three-dimensionally.

Concurrent treatment with high dose steroids (e.g., prednisone >10 mg per day), anti-cancer agents or immunostimulatory drugs was not allowed.

Evaluations. Baseline evaluations included medical history, physical examination, vital signs, KPS, complete blood count with differential, platelet count and prothrombin time, blood chemistry (AP, LDH, AST, ALT, total bilirubin, serum creatinine, electrolytes; magnesium, calcium, total protein, albumin, uric acid and BUN or urea), urine analysis, serology, serum βHCG in women with reproductive potential, disease assessment by X-ray chest and by CT-scan, MRI and/or physical exam, 12-lead electrocardiogram, and pain assessment using the Visual Analog Scale. During treatment, KPS, hematology, blood chemistry, urine analysis, disease assessment (using the same methods as at baseline), and VAS tumor pain assessments were evaluated prior to each new cycle. Patients without disease progression at the end of treatment underwent disease assessments every 2 months until progression and all patients were to be followed for adverse events, pain assessment and quality of life every 2 months until death or initiation of further anti-cancer treatment.

Disease response in treated lesions and in all lesions, irrespective of treatment, was to be evaluated according to SWOG criteria. Complete or partial response was confirmed at least 28 days later (Simon, 1989). Progression-free survival and duration of tumor growth control (TGC) (CR+PR+SD≧3 months, were measured from treatment initiation to progression of treated lesions, initiation of further therapy or death, while overall survival was measured from treatment initiation to death. Adverse events and laboratory abnormalities were graded according to the National Cancer Institute - Common Toxicity Criteria (NCI-CTC) version 1.

Statistical methods. The primary endpoint of this trial was the evaluation of the overall response rate (ORR) obtained in treated lesions. Duration of disease control, progression-free survival, and overall survival were secondary efficacy endpoints. Secondary objectives included safety and impact on cancer morbidity and quality of life. The high-dose trial was randomized, with stratification according to continent (North America versus Europe) applied. Time to event data were analyzed using the Kaplan-Meier method (Kaplan and Meier, 1958). The significance of univariate associations was assessed using the logrank test for time to event data, the Pearson χ² or Fisher exact tests, as appropriate, for categorical data, and the non-parametric Mann-Whitney U test for association of continuous and categorical variables. Continuous variables were categorized using the method of maximization of the χ² statistic. All reported p values are two-sided and a p value <0.05 was taken to indicate significance, without any adjustments for multiplicity. The following variables were analyzed: age, gender, continent, Karnofsky Performance status, UICC disease stage, tumor size and nodal involvement at diagnosis, interval between diagnosis and first relapse (PFI), interval between diagnosis and study entry, type of initial treatment at diagnosis, exposure to chemotherapy during prior therapy, number of relapses after initial treatment, disease extension at baseline (either locally recurrent disease, i.e., recurrence in the neighborhood of the primary tumor, or locoregional relapse), target lesion aspect (whether ulcerative, necrotic, infiltrative or in the field of prior radiation), maximum and summed diameter of treated lesions, evidence of metastatic disease at baseline, baseline disease symptoms (weight loss, dysphagia, pain, asthenia), analgesic consumption, VAS pain scale, baseline laboratory parameters (serum albumin, lactose dehydrogenase, hemoglobin), trial (high-dose versus low-dose), study treatment schedule (3-day versus 6-day), and 3-day high-dose administration versus the other two treatment groups. Parameters associated with efficacy outcomes (response, tumor growth control and overall survival) at a significance level <0.20 were included in multivariate analyses. Logistic regression and Cox regression analysis was employed to determine independent prognostic factors for dichotomized and time to event outcome variables, respectively (Kaplan and Meier, 1958; Anderson et al., 1983).

### Example 2

### Results

Between September 1997 and January 2000, 173 patients entered the two studies (T201 and T202) and 164 were treated in 34 centers in Austria, Canada, Finland, Germany, Spain, Switzerland and the USA. One of the treated patients was considered ineligible due to concomitant bronchogenic carcinoma. Hence, there were 163 eligible treated patients (high-dose trial, 3-day arm 52 patients, 6-day arm 53 patients and low-dose trial 58). Patient characteristics were balanced between the 3 treatment groups (Table 1) with the exception that fewer patients at entry in the low-dose study had received chemotherapy during prior treatment (36% versus 64%, p=0.001) and fewer had locally recurrent disease, as opposed to locoregional relapse (31% versus 51%, p=0.021). Treatment characteristics were similar between the groups. A total of 432 cycles were administered for a median of 2 cycles per patient (range, 1 to 48). Fifty-one patients received a single cycle (29%) while 41 patients (24%) received 3 cycles or more.

Safety. Administration of Ad-p53 was well tolerated with no drug-related deaths or treatment-related grade 4 clinical toxicity. Three patients discontinued treatment for toxicity reasons (2%). The most frequent adverse events were transient local symptoms (48% of patients with low grade injection site pain, reaching grade 3 in 10%) and transient grade 1-2 fever (40% of patients) and chills (17%). The incidence of chills and fever was 34% and 53% in the high dose groups and 2% and 20% in the low dose group).

There was no grade 3-4 hepatotoxicity (alkaline phosphatase, bilirubin, AST and ALT) or coagulopathy in any of the patients. Hematological parameters (leukopenia, neutropenia, anemia, thrombocytopenia and lymphocytopenia) did not differ significantly from those seen at baseline. One case of grade 4 anemia was observed in the high dose arm.

Disease response. A higher overall response rate (CR+PR) was observed in the high dose study (6%) while a lower overall response rate (2%) was observed in patients receiving the lower treatment dose (Table 2). Overall, in the higher dose study, the percentage of patients with objective responses or durable stable disease (>3 months) was 20.0% compared to 14.0% in the lower dose group. These differences between the higher and lower dose groups were not statistically significant. Median progression-free survival in responders was 9.5 months (95% CI, 0.6 to 18.3 months), with 3 patients progression-free after 10 to 43 months follow-up. According to the univariate analysis of prognostic factors for response to intratumoral p53 gene therapy, an interval from diagnosis to first relapse (PFI) >12 months and absence of ulceration or necrosis of target lesions were significantly associated with a higher rate of response (Table 3). In addition to these factors, multivariate analysis also retained absence of pain and absence of target lesions with diameter >2.5 cm as independent prognostic factors for response (data not shown).

Tumor growth control (TGC), defined as CR, PR or SD lasting more than 3 months, was achieved in 18% of patients (95% CI, 13 to 25%) (Table 3). Median duration of growth control was 5.6 months (95% CI, 4.2 to 7.0 months). The factors that were found to be independently associated with an increased probability of achieving TGC were PFI >12 months, prior irradiation of target lesions, and patients having target lesions ≦2.5 cm at baseline.

Survival. At the time of analysis, 98% of the patients had progressed and 93% had died. Five of the 11 patients censored for survival were lost to follow-up. An increased risk of death was found to be associated with predictors of disease aggressiveness, such as shorter initial PFI and locoregional disease extension in addition to poor patient condition—low KPS, baseline weight loss, and biochemical abnormalities (Table 4). Independent prognostic factors at baseline for increased survival were determined to be PFI >12 months, prior chemotherapy, treated lesions having a maximum diameter of 2.5 cm, localized disease, KPS 90-100%, absence of baseline weight loss, and normal serum albumin. The independent prognostic factors for increased risk of death were used to construct a prognostic index for survival in this disease setting. Good prognosis was defined as having 0 or 1 risk factors, moderate prognosis as having 2 risk factors and poor prognosis as the presence of 3 or more risk factors. Median survival in these 3 groups was 10.0 months (95% CI, 8.7 to 11.3 months, 24% of patients), 6.4 months (95% CI, 4.9 to 8.0 months, 34% of patients) and 2.9 months (95% CI, 2.2 to 3.5 months, 42% of patients), respectively. Survival in each group was significantly different from the other two (log rank p≦0.003).

In the higher dose group, median survival was 6.0 months compared to 3.5 months in the lower dose group. In addition, there was a statistically significant correlation between patients receiving prior chemotherapy and increased survival in the high dose group (median survival with prior chemotherapy 7.3 months vs. no prior chemotherapy 4.6 months, p=0.007) that was not observed in the low dose group (median survival with prior chemotherapy 3.3 months vs. no prior chemotherapy 3.4 months, p=0.472). Overall survival was significantly longer in patients experiencing a response (CR or PR), compared to non-responders (unadjusted logrankp=0.001) and in patients with tumor growth control, compared to those with PD, NE or short-term SD as best response (unadjusted logrankp<0.001). A significant survival advantage for patients with response or growth control was still observed in successive analyses excluding patients with a survival less than 3, 6 and 8 months, accounting for any potential basis due to the “guarantee time” effect (Kotwall et al., 1987), in which responders are favored in a survival analysis as guaranteed to live at least until their response is observed. When added to the multivariate analysis, response (CR or PR versus SD>3 months versus SD≦3 months, PD or NE) was retained as an independent prognostic factor for survival, with a significantly decreased risk of death for patients with overall response (HR 0.21, 95% CI, 0.07-0.61, p=0.004) or long-term stabilizations (HR 0.48, 95% CI, 0.27-0.87, p=0.015) compared to non-responders. Patients with CR or PR did not have a significantly decreased risk of death with respect to patients with SD >3 months in the survival analysis adjusted for covariates (p=0.16).

Characterization of patients susceptible to benefit from treatment. On the basis of prognostic factors determined for ORR and TGC, a minimal set of parameters that could define a population most likely to benefit from intra-tumoral p53 gene therapy was identified. The principal prognostic factor for all efficacy outcomes was PFI >12 months, with 31% TGC and 7.8 month survival obtained in these patients (Table 5). The exclusion of patients with ulcerative or necrotic lesions and the restriction of maximum lesion diameter to≦5.0 cm served to exclude patients with a low probability of response to treatment. Alternatively, requiring either prior chemotherapy, target lesions ≦2.5 cm or the absence of baseline pain identifies patients with an increased likelihood of response (Table 5). Depending on the parameters selected to define sub-populations, overall response rates in the range 20% to 30% and TGC rates of 50% to 60% could be obtained (Table 5).

### Example 3

### Discussion

Recurrent disease remains the most common form of treatment failure for patients with SCCHN (Kotwall et al., 1987; Brockstein et al., 2004). Loco-regional recurrence is often associated with severe morbidity due to pain, upper airway obstruction and the resultant difficulties in swallowing and speech. In the majority of cases, recurrent disease is incurable (Kotwall et al., 1987; Brockstein et al., 2004). Palliative surgery is difficult and disfiguring, and re-irradiation is constrained by the limited types of lesions that can be re-treated and the morbidities associated with effective doses. Thus, patients with recurrent SCCHN need novel and less toxic treatments. Currently available therapies provide minimal benefit and result in significant toxicity that can exacerbate local tumor morbidity.

Advances in our understanding of the molecular biology of cancer have identified novel targets for therapeutic development. As the prototypical tumor suppressor gene, p53 plays a critical role in regulating the progression of the cell cycle and induction of apoptosis (Kastan et al., 1995). Abnormalities in p53 cell cycle regulation and apoptotic pathways are among the most common and fundamental molecular mechanisms of cancer pathogenesis and treatment resistance and hence formed the rationale for developing p53 gene transfer for cancer therapy (Gjerset and Sobol, 1997; Hartwell and Kastan, 1994; Kastan et al., 1995; Edelman and Nemunaitis, 2003; Ahomadegbe et al., 1995; Ganly et al., 2000; Zhang et al., 1995; Clayman et al., 1995; Clayman et al., 1998; Clayman et al., 1999; Swisher et al., 1999; Nemunaitis et al., 2000; Peng, 2005). In the vast majority of cancers, abnormal p53 function results in uncontrolled cellular proliferation through loss of cell cycle regulation and treatment resistance from impaired apoptosis in response to therapy. These p53 pathways are impaired in virtually all cancer cells, either by mutation/deletion in the p53 gene or by abnormal regulation of p53 gene expression or function in the absence of p53 gene mutations (Hartwell and Kastan, 1994; Kastan et al., 1995).

Preclinical studies with p53 gene therapy using a replication-incompetent adenoviral vector carrying the wild-type p53 gene have shown that p53 transduction can induce apoptosis and decrease tumor proliferation without adversely affecting normal tissues (Gjerset and Sobol, 1997; Zhang et al., 1995; Clayman et al., 1995). Previous clinical trials of p53 gene transfer were well tolerated and demonstrated a response advantage in nasopharyngeal carcinoma when combined with radiation therapy in earlier stage disease(Clayman et al., 1998; Swisher et al., 1999; Nemunaitis et al., 2000; Peng, 2005).

The present studies were undertaken to evaluate the safety and efficacy of adenoviral p53 gene transfer as monotherapy in patients with locally advanced, recurrent, non-resectable SCCHN. Treatment of a large series of recurrent SCCHN patients with high and low doses of Ad-p53 permitted examination of dose effects and the identification of prognostic factors correlating with p53 gene therapy efficacy. Administration of Ad-p53 was well tolerated and the results suggest dose related association with side effects and survival defining a preferred dose for future administration.

As these clinical trials employed broad selection criteria, the inventors undertook an analysis of prognostic factors to identify patients most likely to benefit from intra-tumoral p53 gene therapy. Their findings indicate that patients with objective tumor responses or long-term tumor stabilization did benefit from study treatment by improved survival. Tumor response and long-term stabilization were retained as independent prognostic factors for survival when added to the multivariate model.

A long progression free interval after initial therapy (at least 12 months) was found to be the major prognostic factor for all efficacy outcomes. This finding is consistent with the molecular profiles of p53 upstream regulators mdm2 and p14ARF that correlate with progression free interval and may influence the therapeutic activity of Ad-p53 (Sano et al., 2000; Kwong et al., 2005). p14ARF exerts its positive effects on p53 activity by blocking the inhibition of p53 by mdm2 (Sano et al., 2000; Kwong et al., 2005). Tumors with intact p14ARF function and normal mdm2 levels would be expected to derive the most benefit from Ad-p53 therapy and these molecular profiles are associated with an increased progression free interval in SCCHN (Sano et al., 2000; Kwong et al., 2005). These observations are consistent with the data from our study indicating that Ad-p53 may have increased activity in patients with a progression free interval >12 months. These individuals are more likely to have an intact p14ARF/mdm2 regulatory axis that would permit Ad-p53 delivered p53 to activate cell cycle arrest and apoptotic pathways.

To the inventor's knowledge, this is the first report of prognostic factors for tumor response and survival for local cancer gene therapy. This analysis extends the findings of previous prognostic factor studies for first-line chemotherapy in SCCHN concerning the importance of PFI, performance status and weight loss (Forastiere et al., 1992; Jacobs et al., 1992; Argiris and Forastiere, 2004; Pivot et al., 2001; Reconado et al., 1991). In contrast to previous findings regarding the negative impact of chemotherapy on subsequent treatments (Pivot et al., 2001), these studies indicate that prior chemotherapy was a positive prognostic factor. In addition, treated lesions in a prior radiation field was also a favorable prognostic factor for tumor growth control. These observations are consistent with the known mechanism of action of p53 gene therapy related to the induction of apoptosis in the presence of DNA damage due to irradiation or chemotherapy resulting in cytotoxicity when p53 function is reactivated Gjerset and Sobol, 1997; Hartwell and Kastan, 1994). The size of treated lesions (≧2.5 cm) was also found to be a favorable prognostic factor for both tumor response and tumor growth control. This may be partially explained by pharmacological levels of p53 inducing anti-angiogenic and immune mediated effects that may have greater activity in smaller lesions. The absence of ulcerated and/or necrotic lesions and the absence of baseline tumor-pain also appeared to identify tumors more suitable for intra-lesional contusugene treatment and were independent factors for response. Combinations of the prognostic factors identified in our study permitted the definition of sub-populations in which tumor response rates in the range 20% to 30% and TGC rates of 50% to 60% could potentially be obtained.

Another important outcome of these studies was that adenoviral p53 gene therapy was well tolerated after long term follow-up in a large series of patients and there were no unexpected adverse events thought to be related to adenoviral p53 administration. The well tolerated nature of p53 gene therapy combined with prognostic factors associating benefit with prior chemotherapy and radiation implies that p53 gene transfer may be utilized to enhance standard cancer therapies without adding significant toxicity. This observation is consistent with the results of previous clinical trials combining p53 gene therapy with standard chemotherapy and radiation (Swisher et al., 1999; Nemunaitis et al., 2000; Peng, 2005).

In conclusion, these studies provide important safety and efficacy data regarding intralesional administration of adenoviral p53 gene therapy in heavily pretreated SCCHN patients. p53 gene therapy was well tolerated and prognostic factors related to tumor growth control and survival were defined. Consistent with the known mechanisms of p53 action on tumor apoptosis in response to DNA damage, prior chemotherapy and radiation treatment were favorable prognostic factors. These findings in a large series of patients extend the results of earlier clinical trials and identify recurrent SCCHN patients most likely to benefit from adenoviral p53 gene therapy.

TABLE 1
Patient Characteristics (% of Patients)

| | High dose | | Low dose |
|---|---|---|---|
| | 3-day | 6-day | 3-day |
| | (N = 52) | (N = 54) | (N = 58) |
| Sex | | | |
| Male | 83 | 70 | 74 |
| Female | 17 | 30 | 26 |
| Median age (years, | 62 (28-86) | 63 (37-89) | 60 (26-90) |
| range) | | | |
| KPS (%) | | | |
| 90-100 | 52 | 51 | 52 |
| 70-80 | 33 | 34 | 40 |
| 50-60 | 13 | 11 | 9 |
| Unknown | 2 | 4 | — |
| Primary anatomic site | | | |
| oral cavity | 35 | 26 | 29 |
| larynx | 25 | 26 | 33 |
| oropharynx | 13 | 21 | 24 |
| hypopharynx | 6 | 9 | 5 |
| nasopharynx | 6 | 4 | 2 |
| neck (unknown | 13 | 6 | 5 |
| primary) | | | |
| Disease stage | | | |
| at diagnosis | | | |
| I-II | 24 | 24 | 30 |
| III | 17 | 25 | 26 |
| IV | 46 | 38 | 41 |
| unknown | 13 | 13 | 3 |
| Initial treatment | | | |
| surgery | 71 | 55 | 55 |
| radiotherapy | 50 | 53 | 55 |
| radiochemotherapy | 21 | 25 | 7 |
| chemotherapy | 2 | 2 | 5 |
| Prior chemotherapy | 65 | 64 | 36 |
| Interval between | | | |
| diagnosis and first | | | |
| relapse (months) | | | |
| median (range) | 13.3 (1.1-21.1) | 12.9 (3.5-96.6) | 10.9 (3.9-94.4) |
| <6 months | 12 | 17 | 6 |
| 6-12 months | 37 | 28 | 53 |
| >12 months | 50 | 51 | 42 |
| Number of relapses | | | |
| after primary treatment | | | |
| 1 | 21 | 25 | 26 |
| 2 | 37 | 26 | 43 |
| 3 | 29 | 32 | 19 |
| >3 | 12 | 13 | 11 |
| Disease extension | | | |
| at baseline | | | |
| locally recurrent | 50 | 51 | 31 |
| locoregional relapse | 50 | 49 | 69 |
| Target lesion aspect | | | |
| ulcerated/necrotic | 33 | 38 | 36 |
| infiltrating | 56 | 60 | 55 |
| irradiated | 81 | 79 | 76 |
| Evidence of metastatic | 19 | 13 | 21 |
| disease | | | |
| Symptoms at baseline | | | |
| pain | 75 | 74 | 81 |
| dysphagia | 19 | 25 | 36 |
| weight loss | 19 | 15 | 17 |
| hemoglobin <10 g/dL | 10 | 9 | 7 |
| albumin <35 g/L | 24 | 32 | 25 |

TABLE 2
Efficacy Outcomes in Eligible Treated Patients

| | | | Low-dose |
|---|---|---|---|
| | High-dose study | | study |
| | 3-day | 6-day | 3-day |
| | (N = 52) | (N = 53) | (N = 58) |
| Best overall response (%) | | | |
| CR | 2 | 2 | — |
| PR | 4 | 4 | 2 |
| SD >3 months | 23 | 6 | 12 |
| SD ≦3 months | 4 | 8 | 3 |
| PD | 48 | 64 | 67 |
| NE | 19 | 17 | 16 |
| Best overall response rate | | | |
| (CR + PR) | | | |
| % of subjects (95% CI) | 6 (1-16) | 6 (1-165) | 2 (0-9) |
| Tumor Growth Control | | | |
| Rate (CR + PR + | | | |
| SD >3 months) | | | |
| % of subjects (95% CI) | 29 (17-43) | 11 (4-23) | 14 (5-23) |
| Progression Free | | | |
| Survival in patients | | | |
| with tumor control | | | |
| Median (95% CI) | 3.7 (2.1-5.3) | 5.1 (0.2-10.0) | 3.8 (0.8-5.8) |
| Progression-free | | | |
| survival (months) | | | |
| median (95% CI) | 1.8 (1.6-2.1) | 1.2 (0.6-1.8) | 0.9 (0.6-1.3) |
| Duration of tumor | | | |
| growth control | | | |
| (months) | | | |
| median (95% CI) | 5.6 (3.5-7.6) | 6.0 (4.2-7.7) | 4.7 (0.0-10.3) |
| Overall survival (months) | | | |
| median (95% CI) | 5.9 (2.5-9.3) | 6.3 (5.2-7.5) | 3.4 (2.5-4.4) |

TABLE 3
Response and Tumor Growth Control,
Univariate and Multivariate Analysis

| | Response | | Tumor growth control | | | |
|---|---|---|---|---|---|---|
| | Uni- | | Uni- | | | |
| | variate | | variate | | | |
| | | Fisher | | Fisher | Multivaiate | |
| | % | P | % | P | OR (95% CI) | p |
| PFI after initial | | | | | | |
| treatment | | | | | | |
| >12 months | 9 | 0.005 | 31 | <0.001 | 9.0 (2.6-30.7) | <0.001 |
| ≦12 months | 0 | | 5 | | | |
| Maximum treated | | | | | | |
| lesion diameter ≦ | | | | | | |
| 2.5 cm | | | | | | |
| no | 10 | >0.1 | 35 | 0.015 | 4.5 (1.4-14.7) | 0.011 |
| yes | 3 | | 14 | | | |
| Target lesions | | | | | | |
| in prior | | | | | | |
| irradiation field | | | | | | |
| yes | 5 | >0.1 | 21 | 0.017 | 6.7 (0.7-62.2) | 0.093 |
| no | 3 | | 3 | | | |
| Pain | | | | | | |
| no | 10 | 0.057 | 31 | 0.028 | NR | |
| yes | 2 | | 14 | | | |
| Ulcerative/ | | | | | | |
| necrotic | | | | | | |
| target lesions | | | | | | |
| no | 7 | 0.049 | 20 | >0.1 | NR | |
| yes | 0 | | 13 | | | |
| KPS | | | | | | |
| 90-100 | 6 | >0.1 | 23 | 0.096 | NR | |
| <90 | 3 | | 12 | | | |
| Number of prior | | | | | | |
| relapses | | | | | | |
| ≧2 | 6 | >0.1 | 21 | 0.026 | NR | |
| <2 | 0 | | 5 | | | |

Abbreviations:
OR, odds ratio;
NR, not retained in multivariate model.

TABLE 4
Overall Survival, Univariate Analysis
and Multivariate Cox Proportional Hazards Model

| | | | Multivariate model | | |
|---|---|---|---|---|---|
| | Univariate analysis | | Hazard ratio | | |
| | Median | Logrank | for risk of death | | |
| | (months) | P | HR | 95% CI | p |
| PFI | | | | | |
| ≦12 months | 3.3 | <0.001 | 1.91 | 1.35-2.72 | <0.001 |
| >12 | 7.8 | | | | |
| Prior chemotherapy | | | | | |
| no | 3.9 | 0.008 | 1.56 | 1.10-2.22 | 0.014 |
| yes | 6.4 | | | | |
| KPS | | | | | |
| <90% | 3.4 | <0.001 | 1.77 | 1.21-2.58 | 0.003 |
| 90-100% | 7.7 | | | | |
| Maximum treated | | | | | |
| lesion diameter | | | | | |
| >2.5 cm | 4.5 | 0.015 | 1.81 | 1.14-2.89 | 0.013 |
| ≦2.5 cm | 8.5 | | | | |
| Baseline weight loss | | | | | |
| yes | 2.7 | <0.001 | 1.74 | 1.10-2.76 | 0.018 |
| no | 5.9 | | | | |
| Albumin | | | | | |
| <35 g/L | 3.5 | <0.001 | 1.64 | 1.06-2.52 | 0.025 |
| ≧35 g/L | 6.4 | | | | |
| Disease extent at | | | | | |
| inclusion | | | | | |
| locoregional | 4.0 | 0.016 | 1.61 | 1.12-2.31 | 0.010 |
| local | 6.9 | | | | |
| Hemoglobin | | | | | |
| <11 g/dL | 3.9 | 0.003 | | NR | |
| ≧11 g/dL | 5.7 | | | | |
| Pain | | | | | |
| yes | 4.3 | 0.017 | | NR | |
| no | 6.9 | | | | |
| Number of prior | | | | | |
| relapses | | | | | |
| <2 | 3.7 | 0.024 | | NR | |
| ≧2 | 5.6 | | | | |
| Ulcerative/necrotic | | | | | |
| target lesions | | | | | |
| yes | 4.3 | 0.024 | | NR | |
| no | 5.1 | | | | |

Abbreviations:
HR, Hazard ratio;
NR, not retained in multivariate model.

TABLE 5
Definition of Populations Susceptible to Benefit from Treatment
(High and Low Dose)

| | Number | | | | |
|---|---|---|---|---|---|
| | of | ORR | TGC | Median PFS | Median OS |
| | patients | (%) | (%) | (months) | (months) |
| All patients (high | 163 | 4 | 18 | 1.5 | 5.1 |
| and low dose | | | | | |
| studies) | | | | | |
| PFI >12 months | 75 | 9 | 31 | 1.8 | 7.8 |
| PFI >12 months | 39 | 15 | 33 | 1.4 | 10.2 |
| and prior | | | | | |
| chemotherapy | | | | | |
| PFI >12 months, | 33 | 18 | 49 | 3.6 | 11.4 |
| and no pain or | | | | | |
| lesion diameter | | | | | |
| ≦2.5 cm | | | | | |
| PFI >12 months, | 21 | 29 | 57 | 4.0 | 13.4 |
| and no ulcerative/ | | | | | |
| necrotic lesions, | | | | | |
| and all target | | | | | |
| lesions ≦5.0 cm, | | | | | |
| and either no pain or | | | | | |
| lesion | | | | | |
| diameter ≦2.5 cm | | | | | |

### Example 4

### Li Fraumeni Treatment

Another group of cancer patients likely to respond to gene therapy are those with inherited predispositions to cancer due to germline mutation and loss of function of the gene utilized for therapy. This Example describes the treatment of a Li-Fraumeni patient with a p53 gene therapy targeted to the molecular defect underlying the pathogenesis and therapy resistance of these malignancies. Li-Fraumeni Syndrome is a rare autosomal dominant disorder which predisposes individuals to a variety of malignancies including breast cancer, sarcoma, brain tumors, leukemias, lung cancer, and other cancers. The genetic basis of this syndrome resides in the inherited germline mutations in one p53 suppressor allele (Li and Fraumeni, 1969a; 1969b; Li et al., 1988). In addition to pathogenesis, defects in p53 mediated apoptotic pathways contribute to the eventual resistance of these tumors to standard radiation and chemotherapy. In a tumor refractory to standard treatments, adenoviral p53 gene therapy resulted in a complete remission by PET scan and was associated with improvement of tumor related symptoms.

The Li-Fraumeni Syndrome is an inherited genetic disorder characterized by familial clustering of multiple malignancies predominantly including sarcomas, breast cancers, brain tumors and adrenocortical carcinomas. In this syndrome, there are an inordinate number of primary cancers often with initial occurrence at a young age. These tumors typically become refractory to standard treatment and result in early death. The genetic basis of this syndrome is a germ line mutation in one p53 suppressor allele. Hence, treatment of Li-Fraumeni tumors with p53 gene transfer represents a novel, prototypical targeted cancer therapy for these neoplasms. The following report describes the treatment of a Li-Fraumeni patient with adenoviral p53 gene therapy.

Advexin is a non-replicating serotype adenovirus which contains a wild-type p53 gene sequence. It is a first generation viral vector in which a cytomegalovirus promoter drives expression of p53. An E1 deletion is constructed to inhibit replication. Extensive safety has been demonstrated clinically (Zhang et al., 1995; Clayman et al., 1998; Nemunaitis et al., 2000).

To the inventors' knowledge, the following report describes the first treatment of a patient with Li-Fraumeni Syndrome with adenoviral p53 gene therapy.

Patient History. The patient is a 25 year old woman from a Li-Fraumeni family who presented with abdominal pain, anorexia and vomiting leading to the diagnosis of granulosa cell tumor. p53 sequencing of DNA from her peripheral blood cells confirmed the presence of a germ line p53 abnormality with a codon 151delG frameshift mutation in exon 4 resulting in a downstream stop codon. She was initially treated with oophorectomy followed by five cycles of bleomycin, cisplatin and vinblastine. She subsequently developed embryonal carcinoma of the vagina two years later and was treated with cisplatin, etoposide and ifosfamide initially and then switched from ifosfamide to taxol after development of encephalopathy. She experienced progressive disease with bone invasion after three cycles of treatment and received pelvic radiotherapy (45Gy) and whole brain radiation when she developed CNS metastases.

The patient's pelvic disease continued to progress with resultant development of lower extremity edema. Her physicians did not believe she would benefit from additional conventional chemotherapy, radiation or surgery and she was referred for experimental treatment with p53 gene therapy under a local IRB approved compassionate use protocol.

Results and Discussion. The patient has received 4 intratumoral injections of Advexin, i.e., INGN 201, at a dose of 2×10¹² vp/injection twice weekly on days 2 and 4 of week 1 every 28 days×2 and is currently receiving weekly injections at a dose of 2×10¹² vp/injection. The patient has tolerated the treatment well and has not experienced any grade 3 or 4 adverse vector-related effects. A fusion PET/CT scan following 4 treatment injections has shown complete resolution of 2,3-FDG uptake at the injection sites (see FIG. 1).

The patient's pelvic neoplasm was treated by intratumoral injection of contusugene (Advexin), a replication-defective adenoviral vector containing a minigene expression cassette comprised of a CMV promoter, wild-type human p53 cDNA, and an SV40 polyadenylation signal inserted into the E1-deleted region of adenovirus serotype 5. She received intratumoral adenoviral p53 (Advexin, i.e., INGN 201) at a dose of 2×10¹² viral particles/injection twice weekly on days 2 and 4 of week 1 then every 28 days for two additional treatments. The patient has tolerated the therapy well and side effects were limited to grade 1 injection site pain and fever. A fusion PET/CT scan following 4 intratumoral injections of adenoviral p53 gene therapy has shown complete resolution of 2,3-FDG uptake in the treated tumor compared to the pre-treatment evaluation (see FIG. 1). This response was associated with a significant change in tumor resistance to needle insertion and improvement in lower extremity edema.

Immunotherapies. Immunotherapies may also be employed to treat, prevent or delay the onset or recurrence of Li-Fraumeni tumors and other hyperplasias that result from genetic mutations. In this embodiment, immunotherapy may be utilized when the mutated hyperplasia related gene results in its increased protein expression in the hyperplastic tissue while normal tissues express low levels of this target hyperplasia antigen. This difference in expression levels between the hyperplastic and normal tissues permits destruction of the hyperplastic tissue by immune mechanisms that recognizes the higher target protein levels and attacks hyperplastic tissues but does not significantly recognize and damage normal tissues.

In this embodiment, the individual patient is first tested to determine eligibility by confirming that there are high levels of the target protein in the hyperplastic tissues but low levels in normal tissue. This determination may be made by one of ordinary skill in the art by immunoassays or molecular assays performed on hyperplastic tissues like tumors and normal tissues like blood cell or skin. An eligible patient is then treated with an immunotherapy directed against the target antigen.

There are numerous tumor immunotherapy methods involving tumor associated antigens that may be applied by one of ordinary skill in the art to generate an immune response against the hyperplastic target antigen. In a particular embodiment, for the management of patients with Li-Fraumeni Syndrome, immunotherapies targeted to p53 may be employed as described in U.S. Publications 2005/0171045 and 2005/0226888, incorporated herein by reference.

The following protocol describes a protocol for the treatment of Li-Fraumeni patients. One of ordinary skill in the art would know that the protocol serves as a general guide to the treatment of such patients that may be modified by combinations with other therapies known to be useful in the treatment of cancers.

Exclusion Criteria. Patients may be excluded from treatment if they have been subject to chronic treatment with non-steroidal anti-inflammatory medication or aspirin (except as cardio-prophylaxis; 81 mg/day).

Formulation and Storage. INGN 201 (also known as Adenoviral-p53, ADVEXIN) is manufactured by Introgen Therapeutics Inc. It is provided as a frozen viral suspension in phosphate buffered saline (PBS) containing 10% (v/v) glycerol as a stabilizer. Each vial will contain 1×10¹² vp in 1 mL. Prior to dilution, the vials of INGN 201 should remain frozen at -60° to -80° C. Dose preparation should take place under a BSL2 hood. Drug handling precautions for cytotoxic drugs, universal precautions for infectious material and biological safety level 2 (BSL2) guidelines should be followed. Avoid contact or inhalation, and use appropriate protective clothing. Goggles, masks, gloves and gowns are recommended. Adherence to Institutional, Country, State and Local regulations is required.

After removal from the freezer, INGN 201 must be injected within 8 hours if kept cold; i.e., at 2°-10° C. or within 2 hours if kept at room temperature, i.e., 15°-25° C. Dose preparation should take place under a BSL2 hood.

Dosage and Administration. Prior to INGN 201 injection, patients may be pre-medicated with local or systemic analgesics at the Investigator's discretion based on the patient's pre-existing pain or anticipated pain from the injection. INGN 201 will be administered at 2×10¹² vp/injection on days 2 and 4 of the first 28 day cycle. Thereafter, if no INGN 201 related grade 3 or 4 adverse events have been observed, INGN 201 will be administered weekly, four weekly injections defining 1 cycle.

INGN 201 will be delivered by multiple injections and/or deposit spots with a fine needle (no finer than 27 gauge) directly into the lesion, in a clockwise circumferential pattern through a central penetration site to cover the entire lesion. Attention must be paid to adequately infiltrate tumor margins.

It is recommended that INGN 201 be administered as soon as possible after reconstitution. INGN 201 may be warmed to room temperature prior to injection (within 30 minutes). The time between removal from the storage freezer to administration should be no more than 8 hours if kept cold (between 2° and 10° C.) or within 2 hrs if kept at room temperature (15°-25° C.). No isolation will be required. During treatment, or the STFU period, patients will be instructed to wash their hands after urine or stool voiding, use disposable paper tissues when coughing or sneezing, avoid contact with former tissue transplant recipients or persons known to them as suffering from severe immunodeficiency disease (either congenital or acquired).

Dosing Regimen. Duration of one cycle is 28 days (4 weeks). Day one of the cycle will be the first day of study treatment administration. Patient will be treated for 6 cycles unless there is documented progression of the target lesions, overall disease progression or unacceptable adverse events. It is important that the Investigator, especially during the first two cycles, attempts to determine if the enlargement of an injected lesion is due to disease progression or swelling, which may be caused by a tumor response. It has been reported that these lesions may show significant swelling and increased or new necrosis after being treated with the first or second cycle of injections. Treatment beyond 6 cycles without evidence of disease progression can be considered after re-evaluation and assessment of toxicity.

Treatment Delay. If the patient experiences significant toxicity which did not resolve during the treatment free interval, re-treatment of administration of INGN 201 may be delayed for a maximum of 2 weeks. If during that period, there is recovery either to the screening value for a preexisting sign and symptom or to grade ≧1, the next cycle will be performed with administration of INGN 201 as originally planned. In the absence of recovery, the patient will be removed from treatment.

Toxicity. Toxicities will be graded and reported according to the NCI Common Toxicity Criteria for Adverse Events (CTCAE) Version 3.0 (see //ctep.info.nih.gov).

Schedule of Assessments. Signed Informed Consent should be obtained before any study specific procedures are performed. The screening evaluation is defined as a study specific assessment of the patient status prior to any study treatment. The following evaluations should be performed and results obtained within 14 days prior to treatment initiation (except as indicated below): (i) Complete Medical History: including diagnosis and staging of primary cancer, previous illness, concomitant medications, prior anti-cancer therapy and existing disease related signs and symptoms; (ii) Physical Examination; (iii) Vital signs: including temperature, pulse, blood pressure; (iv) Height and weight: height is measured only during the screening period, weight will be recorded immediately before drug administration on day 1; (v) Karnofsky Performance Status; (vi) Serology: including HIV 1, HIV 2, Hepatitis B surface antigen, Hepatitis C antibody; (vii) Serum PHCG: if female of childbearing potential; (viii) Hematology: including CBC with differential and platelet count, prothrombin time and partial thromboplastin time; (ix) Biochemistry: including alkaline phosphatase, AST/SGOT, ALT/SGPT, LDH, total bilirubin, serum creatinine and creatinine clearance (actual or estimated), glucose, phosphorus, electrolytes, magnesium, calcium, protein, albumin, uric acid and BUN; (x) Urinalysis: routine U/A; (xi) Chest X-ray: Posterior/Anterior (PA) and Lateral; (xii) Tumor Assessment: including MRI or CT scan of tumor lesions in all disease sites; (xiii) Biodistribution/Biosafety: plasma and urine sampling should be performed within 3 days before the first INGN 201 administration; (xiv) antibody testing: serum should be obtained within 3 days before the first INGN 201 for neutralizing antibodies against Ad5, anti-adenoviral, and anti p53 antibody testing; (xv) p53 mutation status: it is desirable for tumor specimens to be obtained from all patients prior to study treatment to evaluate p53 mutation status

Tumor Response. The following designations are used in evaluating target lesions: Complete Response (CR)—disappearance of all target lesions; Partial Response (PR)—at least a 30% decrease in the sum of the longest diameter (LD) of target lesions, taking as reference the baseline sum LD; Progression (PD)—At least a 20% increase in the sum of LD of target lesions, taking as reference the smallest sum LD recorded since the treatment started or the appearance of one or more new lesions; Stable Disease (SD)—Neither sufficient shrinkage to qualify for PR nor sufficient increase to qualify for PD taking as reference the smallest sum LD since the treatment started. A decrease of tumor 2,3-FDG uptake on fusion PET/CT scans following treatment injections is an additional measure of treatment response.

Evaluation of Non-Target Lesions. The following designations are used in evaluating non-target lesions: Complete Response—disappearance of all non-target lesions and normalization of tumor marker level; Incomplete Response/Stable Disease—persistence of one or more non-target lesions (non-CR) and/or maintenance of tumor marker level above the normal limits; Progressive Disease—appearance of one or more new lesions, unequivocal progression of existing non-target lesions. Note: If tumor markers are initially above the upper normal limit, they must normalize for a patient to be considered in complete clinical response.

Evaluation of Best Overall Response. The best overall response is the best response recorded from the start of the treatment until disease progression/recurrence (taking as reference for progressive disease the smallest measurements recorded since the treatment started). The patient's best response assignment will depend on the achievement of both measurement and confirmation criteria (see section Evaluation of Target Lesions).

### Example 5

### P53 Expression in Cancers Predicts Gene Therapy Outcome

1. Introduction

This data that follow demonstrate that p53 expression in SCCHN is a statistically significant predictor of Advexin gene therapy outcome. Specifically, patients whose cancers expressed detectable levels of p53 using immunohistochemistry (p53 is typically not detectable in normal cells using immnuohistoochemistry) correlated with a therapeutic response of the cancer to the Advexin therapy. This study included 28 pretreatment tumor samples from patients with advanced SCCHN enrolled in Phase 2 clinical trials for Advexin. Most patients participated in the T-201 and T-202 studies; one patient was from the T-207 trial, a non-U.S. IND study evaluating the same patient population with the higher Advexin dose Tumor samples from every patient treated were requested from all clinical sites in the studies and analysis was performed on all patients for whom tumor samples were available at the time of the evaluation (i.e., samples were collected randomly and there was no selection of patients). Molecular analysis was performed independently by a contract laboratory by personnel blinded to clinical outcome data.

Expression of endogenous normal p53 protein in normal tissues is tightly regulated at a low level, and is undetectable by immunohistochemistry. In contrast, in malignant tissues p53 protein is often over-expressed and readily detectable, reflecting defects in the regulation and function of the p53 pathway. Evaluation of the literature showed a similar pattern and frequency for positive p53 immunostaining in a broad spectrum of human tumors that were observed in this study. The inventors reviewed 21 published reports evaluating p53 immunostaining in SCCHN. These studies involved >1,700 SCCHN patient samples and the cutoff signal used to determine positive versus negative staining in the literature averaged 17%. Based upon these analyses, the inventors selected ≧20% immunostaining of nuclear p53 to define “p53-positive” tumors. This resulted in 57% p53 positivity in this study which is very similar to the overall frequency of 53±10% p53 positivity of SCCHN tumors identified in the literature review.

2. Methods and Results

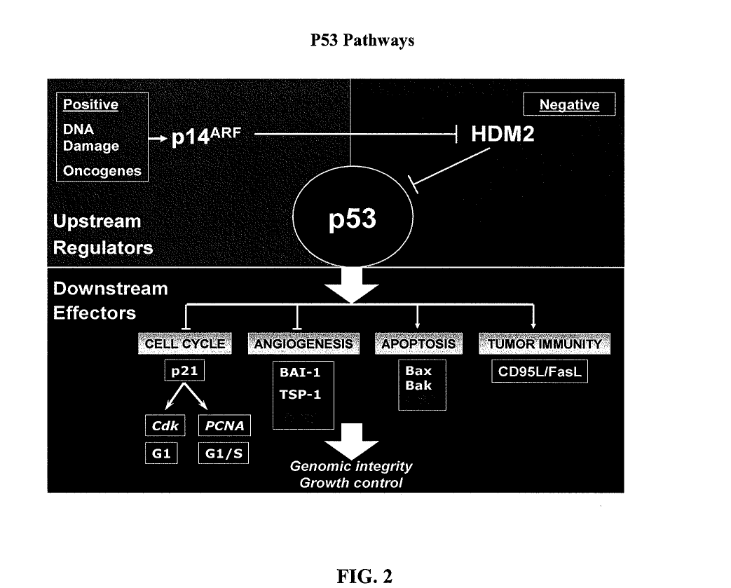

To assess the value of p53 pathway abnormalities in predicting Advexin efficacy, immunohistochemical analyses of p53 protein expression, and several other proteins in the p53 pathway, were performed under GLP conditions on pretreatment tumor samples from patients with recurrent locally advanced SCCHN treated with Advexin. The proteins selected for analysis are intimately linked to regulation of p53 regulation and function, i.e., p14ARF, HDM2, bcl-2, survivin and phospho-ser15-p53. The biochemical basis for selection of these specific markers is provided in FIG. 2.

Using ≧20% p53 staining as criterion for positivity, the data show that 57% of SCCHN patients showed abnormal p53 over-expression; this value is consistent with the p53 positivity rate of 53±10% found with >1,700 SCCHN patients. The inventors also evaluated 46 additional SCCHN tumor specimens obtained from a tumor bank and observed a similar pattern of p53 staining and frequency of p53 positivity. When the two datasets (Advexin patients and control SCCHN specimens from tumor bank) were compared using either cutoff values of either ≧20% or ≧50% p53 staining, no significant differences in p53 distribution were observed between the datasets, indicating that the distribution of p53 immunostaining in the Advexin patient group is comparable to the control group.

Abnormal p53 protein over-expression in pre-treatment tumors was correlated with increased Advexin Locoregional Disease Control and survival in the subset of the Phase 2 SCCHN patients for whom tumor samples were available (n=28; Table 6). Immunohistochemical staining was performed using the DO-7 monoclonal antibody, which detects both wild-type and mutant protein.

As shown in Table 6, abnormal expression of p53 by immunohistochemistry in pre-treatment tumors had a statistically significant correlation with locoregional disease control following Advexin therapy. Seventy-five % (12/16) of patients with p53 positive tumors demonstrated locoregional disease control, compared to only 18% with p53 negative tumors (p=.0063, Fisher's Exact Test).

TABLE 6
Statistically Significant Correlation of p53 Abnormality by
Immunohistochemistry with Locoregional Disease Control
following Advexin Treatment in Recurrent SCCHN Significant
Table 6: Fisher's Exact Test

| P53 Protein Level | Locoregional Disease Control |
|---|---|
| Positive (≧20%) | 75% (12/16) |
| Negative (<20%) | 18% (2/11) |

p-value = 0.0063; Fishers Exact Test

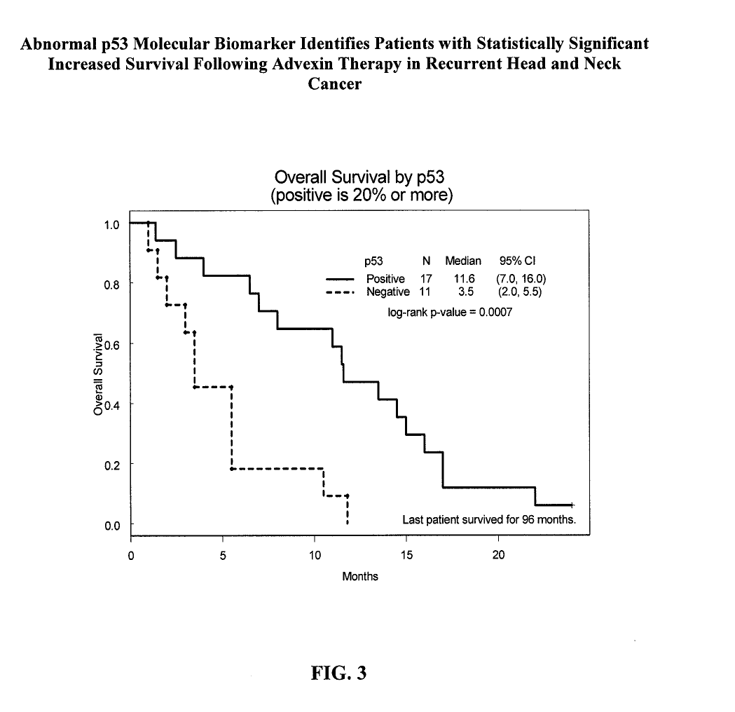

With respect to survival, Advexin treatment of recurrent SCCHN patients with p53 abnormalities had statistically significant increased median survival compared to those whose pre-treatment tumors did not over-express p53 protein (median survival 11.6 vs. 3.5 monthsp<0.0007; Log Rank Test) (FIG. 3).

The p53 signaling pathway involves a complex interplay of positive and negative regulators. Although the p53 protein serves as the pivotal node, it is the integration of this complex network that determines cellular response to stress. p53 is regulated by phosphorylation, acetylation, and sumoylation of specific residues, and by control of the protein half life and degradation. Of these p53 modifications, Serine-15 phosphorylation is key to the activation of multiple signaling pathways induced by DNA damage (Shieh 1997, Tibbetts 1999). A number of upstream regulators and many downstream targets of p53 activity have been identified. The inventors selected two key upstream proteins predicted to modulate p53 activity (HDM2 and p14^{ARF}) and two apoptotic regulatory proteins (Bcl2 and survivin) for further analysis by IHC. In addition, the inventors specifically examined the activated form of p53 protein which is phosphorylated on Serine-15.

HDM2 (termed MDM2 in mice) binds wild-type p53 and targets its destruction via the protein degradation (proteosome) pathway. Phosphorylation of p53 at Serine-15 reduces the direct interaction of p53 with HDM2 and stabilizes p53. Thus HDM2 is a negative regulator of p53 and HDM2 gene amplification or overexpression is associated with p53 inactivation and tumor development. p14^{ARF} is a positive regulator of p53 activity and functions by blocking the inhibition of p53 by HDM2. p14^{ARF} gene alterations have been associated with SCCHN carcinogenesis. Both Bc12 and survivin are members of the inhibitor of apoptosis (IAP) family and are believed to play central roles in the progression and resistance to therapy of multiple tumor types. Increased expression of Bc12 and survivin has been documented in many tumor types, including SCCHN. Elevated Bc12 and survivin levels were shown to be negative predictors of patient survival and inversely correlated with wild type p53 status and apoptosis (Pan et al., 2006; Atikcan et al., 2006; Rosato et al., 2006; Parker et al., 2006; Gallo et al., 1999; Sharma et al., 2004; Nakano et al., 2005). Wild-type p53 serves as a negative transcriptional regulator of survivin and high levels of survivin in tumor cells indicates loss of p53 tumor suppressor function (Xia, 2006; Nakano, 2005).

In this study, the invnetors have shown that detection of p53 abnormalities by immunohistochemistry can serve as a molecular biomarker for predicting tumor response and survival benefit after Advexin treatment in patients with recurrent, locally advanced SCCHN. Tumors demonstrating overexpression of p53 reflect abnormalities of the p53 pathyway that can be corrected by Advexin administration. These findings are consistent with the poor survival, lack of response to chemotherapy or radiotherapy that defines the unmet medical needs of patients with p53 abnormalities that are benefited most by Advexin therapy. The inventors' published data indicates that delivery of pharmacologic levels of wild type p53 protein by Advexin restores functional p53 signaling and promotes the apoptotic pathway in treated tumors. Therefore, the identification of abnormal p53 by immunohistochemistry is a predictive biomarker for Advexin activity in this group of patients and reflects the aberrant molecular signaling pathway that is targeted by Advexin. In addition, these findings indicate that Advexin therapy provides a high level of therapeutic patient benefit for the patient subpopulation most at risk for poor clinical outcome in this disease.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### IX. REFERENCES

The following references, to the extent that they provide exemplary procedural or other details supplementary to those set forth herein, are specifically incorporated herein by reference:
U.S. Pat. No. 4,415,723
U.S. Pat. No. 4,458,066
U.S. Pat. No. 4,659,774
U.S. Pat. No. 4,682,195
U.S. Pat. No. 4,683,202
U.S. Pat. No. 4,797,368
U.S. Pat. No. 4,816,571
U.S. Pat. No. 4,879,236
U.S. Pat. No. 4,946,778
U.S. Pat. No. 4,959,463
U.S. Pat. No. 5,139,941
U.S. Pat. No. 5,141,813
U.S. Pat. No. 5,264,566
U.S. Pat. No. 5,302,523
U.S. Pat. No. 5,322,783
U.S. Pat. No. 5,354,855
U.S. Pat. No. 5,384,253
U.S. Pat. No. 5,428,148
U.S. Pat. No. 5,464,765
U.S. Pat. No. 5,538,877
U.S. Pat. No. 5,538,880
U.S. Pat. No. 5,550,318
U.S. Pat. No. 5,554,744
U.S. Pat. No. 5,563,055
U.S. Pat. No. 5,574,146
U.S. Pat. No. 5,580,859
U.S. Pat. No. 5,589,466
U.S. Pat. No. 5,602,244
U.S. Pat. No. 5,610,042
U.S. Pat. No. 5,645,897
U.S. Pat. No. 5,656,610
U.S. Pat. No. 5,670,488
U.S. Pat. No. 5,672,344
U.S. Pat. No. 5,677,178
U.S. Pat. No. 5,702,932
U.S. Pat. No. 5,705,629
U.S. Pat. No. 5,736,524
U.S. Pat. No. 5,747,469
U.S. Pat. No. 5,747,869
U.S. Pat. No. 5,780,448
U.S. Pat. No. 5,789,215
U.S. Pat. No. 5,795,715
U.S. Pat. No. 5,871,986
U.S. Pat. No. 5,879,703
U.S. Pat. No. 5,888,773
U.S. Pat. No. 5,889,136
U.S. Pat. No. 5,932,210
U.S. Pat. No. 5,945,100
U.S. Pat. No. 5,981,225
U.S. Pat. No. 5,981,274
U.S. Pat. No. 5,994,136
U.S. Pat. No. 5,994,624
U.S. Pat. No. 6,013,516
U.S. Pat. No. 6,017,524
U.S. Pat. No. 6,069,134
U.S. Pat. No. 6,136,594
U.S. Pat. No. 6,143,290
U.S. Pat. No. 6,210,939
U.S. Pat. No. 6,296,845
U.S. Pat. No. 6,410,010
U.S. Pat. No. 6,511,184
U.S. Pat. No. 6,511,847
U.S. Pat. No. 6,555,376
U.S. Pat. No. 6,627,190
U.S. Pat. No. 6,797,704
U.S. Patent App. 20050171045
U.S. Patent App. 2005/0226888
U.S. Patent App. 2004/0213764
U.S. Patent App. 2002/0077313
U.S. Patent App. 2002/0028785
U.S. Patent App. 2002/0006914
U.S. Patent App. 20050143336
U.S. Patent App. 20050196343
U.S. Patent App. 20050203047
U.S. Patent App. 20050260276
U.S. Patent application Ser. No. 09/351,778
Ahomadegbe et al., Oncogene, 10(6):1217-1227, 1995.
Aksentijevich et al., Hum. Gene Ther., 7(9):1111-1122, 1996.
Anderson etal., J. Clin. Oncol., 1(11):710-719, 1983.
Argiris et al., Cancer, 101(10):2222-2229, 2004.
Atikcan et al., Respir. Med.,100:2220-2226, 2006.
Ausubel et al., In: Current Protocols in Molecular Biology, John, Wiley & Sons, Inc, New York, 1996.
Baichwal and Sugden, In: Gene Transfer, Kucherlapati (Ed.), NY, Plenum Press, 117-148, 1986.
Bakhshi et al., Cell, 41(3):899-906, 1985.
Bargonetti et al., Cell, 65(6):1083-1091, 1991.
Batterson and Roizman, J. Virol., 46(2):371-377, 1983.
Bernstein et al., Nature, 409:363-366, 2001.
Bittner et al., Methods in Enzymol, 153:516-544, 1987.
Blomer et al., J. Virol., 71(9):6641-6649, 1997.
Blumberg et al., Cell, 104(1):9-19, 2001.
Bond et al. Cell, 19:591-602, 2004.
Bosher and Labouesse, Nat. Cell. Biol., 2:E31-E36, 2000.
Brennecke et al., Cell, 113:25-36, 2003.
Brockstein et al., Ann. Oncol., 15(8):1179-1186, 2004.
Cancer: Principles and Practice of Oncology (7th Ed.), 2004.
Cancer: Principles and Practice of Oncology Single Volume (CD-ROM), Devita et al. (Eds.), Lippencott, 2001.
Caplen et al., Gene, 252(1-2):95-105, 2000.
Casey et al., Oncogene, 6(10):1791-1797, 1991.
Chen and Okayama, Mol. Cell Biol., 7(8):2745-2752, 1987.
Clayman et al., Cancer Res., 55(14): 1-6, 1995.
Clayman et al., Clin. Cancer Res., 5(7):1715-1722, 1999.
Clayman et al., J. Clin. Oncol., 16(6):2221-2232, 1998.
Cleary and Sklar, Proc. Natl. Acad. Sci. USA, (21):7439-7443, 1985.
Clearyetal., J. Exp. Med., 164(1):315-320, 1986.
Clinical Oncology (3rd Ed.), Martin (Eds.), 2004.
Cook etal., Cell, 27:487-496, 1981.
Cotten et al., Proc. Natl. Acad. Sci. USA, 89(13):6094-6098, 1992.
Couch et al., Am. Rev. Resp. Dis., 88:394-403, 1963.
Curiel, Nat. Immun., 13(2-3):141-164, 1994.
Dameron et al. Science 265:1582-1584, 1994.
De Waal Malefyt et al., J. Exp. Med., 174(4):1209-1220, 1991.
De Vries et al., Proc Natl Acad Sci USA. 99(5):2948-53, 2002.
DeLuca et al., J. Virol., 56(2):558-570, 1985.
Doench et al., Genes Dev., 17:438-442, 2003.
Dumoutier et al., J. Immunol., 164(4):1814-1819., 2000.
Edelman and Nemunaitis, Curr. Opin. Mol. Ther., 5(6):611-617, 2003.
Ekmekcioglu et al., Int. J. Cancer, 94(1):54-59, 2001.
Ekmekcioglu et al., Melanoma Res., 9(3):261-272, 1999.
Elbashir et al., EMBO J., 20:6877-6888, 2001.
Elbashir et al., Genes Dev., 5(2):188-200, 2001.
El-Deiry et al. Cell 75:817-825, 1993.
Elroy-Stein et al., Proc. Natl. Acad. Sci. USA, 86(16):6126-6130, 1989.
EP 266 032
Fechheimer, et al., Proc. Natl. Acad. Sci. USA, 84:8463-8467, 1987.
Feigner et al., Proc. Natl. Acad. Sci. USA, 84(21):7413-7417, 1987.
Fields and Jang, Science, 249(4972):1046-1049, 1990.
Fire et al., Nature, 391(6669):806-811, 1998.
Forastiere etal., J. Clin. Oncol., 10(8):1245-1251, 1992.
Forster and Symons, Cell, 49(2):211-220, 1987.
Fraley et al., Proc. Natl. Acad. Sci. USA, 76:3348-3352, 1979.
Froehler et al., Nucleic Acids Res., 14(13):5399-5407, 1986.
Gabizon et al., Cancer Res., 50(19):6371 -6378, 1990.
Gallagher et al., Genes Immun., 1(7):442-450, 2000.
Gallo et al., Cancer, 75(8):2037-2044, 1995.
Gallo et al., Int. J. Cancer, 84(6):573-579, 1999.
Ganly et al., Br. J Cancer, 82(2):392-398, 2000.
Geisler et al., Clinical Cancer Research, 8:3445 2002
Gene Therapy Protocols (Methods in Molecular Medicine), Morgan (Ed.), Humana Press, 2002.
Gene Therapy: Treating Disease by Repairing Genes,Panno (Ed.), New Biology, 2004.
Gerlach et al., Nature (London), 328:802-805, 1987.
Ghosh and Bachhawat, In: Liver Diseases, Targeted Diagnosis and Therapy Using Specific Receptors and Ligands, Wu et al. (Eds.), Marcel Dekker, NY, 87-104, 1991.
Gjerset and Sobol, In Encyclopedia of Cancer, Bertino (Ed.), Academic Press, 1785-1791, 1997.
Glorioso et al., Mol. Biotechnol., 4(1):87-99, 1995.
Gomez-Foix et al., J. Biol. Chem., 267:25129-25134, 1992.
Gopal, Mol. Cell Biol., 5:1188-1190, 1985.
Graham and Prevec, Biotechnology, 20:363-390, 1992.
Graham and Van Der Eb, Virology, 52:456-467, 1973.
Graham et al, J. General Virology, 36:59-74, 1977.
Grishok et al., Cell, 106:23-34, 2001.
Grishok et al., Science, 287:2494-2497, 2000.
Grunhaus and Horwitz, Seminar in Virology, 3:237-252, 1992.
Harland and Weintraub, J. Cell Biol., 101(3):1094-1099, 1985.
Harper et al., Cell 75:805-816, 1993.
Harris et al. Cancer Gene Ther. 3(2):121-30, 1996.
Hartwell and Kastan, Science, 266(5192):1821-1828, 1994.
Herz and Gerard, Proc. Natl. Acad. Sci. USA, 90:2812-2816, 1993.
Herz and Roizman, Cell, 33(1):145-151, 1983.
Holland and Holland, J. Biol Chem, 255(6):2596-605, 1980.
Hollstein et al., Science, 253(5015):49-53, 1991.
Honess and Roizman, J. Virol., 14(1):8-19, 1974.
Honess and Roizman, J. Virol., 16(5):1308-1326, 1975.
Horio et al., Cancer gene ther 7(4):537-44, 2000.
Hutvgner and Zamore, Science, 297:2056-2060, 2002.
Hutvgner et al., Science, 293:834-838, 2001.
Jacobs et al., J. Clin. Oncol., 10(2):257-263, 1992.
Jiang et al., Oncogene, 11(12):2477-2486, 1995.
Jiang et al., Proc. Natl. Acad. Sci. USA, 93(17):9160-9165, 1996.
Joyce, Nature, 338:217-244, 1989.
Jung et al,, Biochem. Biophys. Res. Commun., 336(4):1164-1171, 2005.
Kaeppler et al., Plant Cell Reports, 9:415-418, 1990.
Kaneda et al., Science, 243:375-378, 1989.
Kaplan and Meier, J. Am .Stat. Assoc., 53:457-481, 1958.
Kastan et al., Cancer Metastasis Rev., 14(1):3-15, 1995.
Kato et al, J. Biol. Chem., 266:3361-3364, 1991.
Kataoka, et al., Oncogene,. 19(12): 1589-95, 2000.
Kelleher and Vos, Biotechniques, 17(6):1110-7, 1994.
Kerr et al., Br. J. Cancer, 26(4):239-257, 1972.
Ketting et al., Genes Dev., 15(20):2654-2659, 2001.
Ketting et al., Cell, 99(2): 133-141, 1999.
Ketting et al., Genes Dev., 15:2654-2659, 2002.
Kim and Cook, Proc. Natl. Acad. Sci. USA, 84(24):8788-8792, 1987.
Knapp et al., Atherosclerosis, 152(1):217-227, 2000.
Knight and Bass, Science, 293:2269-2271, 2001.
Kotenko et al., Proc. Natl. Acad. Sci. USA, 97(4):1695-1700, 2000.
Kotwall et al., Am. J. Surg., 154(4):439-442, 1987.
Kwong et al., Clin. Cancer Res., 11(1 1):4107-4116, 2005.
Ladner et al. Cancer Res. 60:3493-503, 2000.
Lane et al. Nature. 2;358(6381):15-6, 1992.
Laughlin et al., J. Virol., 60(2):515-524, 1986.
Lee et al., Cancer Res 61: 837-841, 2001.
Le Gal La Salle et al., Science, 259:988-990, 1993.
Lebkowski et al., Mol. Cell. Biol., 8(10):3988-3996, 1988.
Lee et al., Cell, 75:843-854, 1993.
Levrero et al., Gene, 101: 195-202, 1991.
Li and Fraumeni, Jr., Ann. Intern. Med., 71(4): 747-752, 1969.
Li and Fraumeni, Jr., J. Natl. Cancer Inst., 43(6): 1365-1373, 1969.
Li et al., Cancer Res., 48(18): 5358-5362, 1988.
Lin and Avery, Nature, 402:128-129, 1999.
Liu et al., Cancer Res., 55(14):3117-3122, 1995.
Lozano and Zambetti, Cancer Cell, 8(1):3-4, 2005.
Lozano and Zambetti J. Pathol. 205(2):206-20, 2005b.
Macejak and Samow, Nature, 353:90-94, 1991.
Mann et al., Cell, 33:153-159, 1983.
Martin et al., Nature, 345(6277):739-743, 1990.
Matsumura et al., Oncology, 53(4):308-312, 1996.
McKaig et al., Head Neck, 20(3):250-265, 1998.
McLaughlin et al., J. Virol., 62(6):1963-1973, 1988.
Mercer, Critic. Rev. Eukar. Gene Express. 2:251-263, 1992.
Michel and Westhof, J. Mol. Biol., 216:585-610,1990.
Mietz et al., EMBO J., 11(13):5013-5020, 1992.
Miller etal., Am. J. Clin. Oncol., 15(3):216-221, 1992.
Montgomery et al., Proc. Natl. Acad. Sci. USA, 95:15502-15507, 1998.
Mourelatos et al., Genes. Dev., 16:720-728, 2002.
Mulderetal., Br. J. Cancer, 71(6):1257-1262, 1995.
Muzyczka, Curr. Topics Microbiol. Immunol., 158:97-129, 1992.
Nabel et al., Science, 244(4910):1342-1344, 1989.
Naldini et al., Science, 272(5259):263-267, 1996.
Nakano et al., Int. J. Oncol., 27:1215-1221, 2005.
Nemunaitis et al., J. Clin. Oncol., 18(3):609-622, 2000.
Nemunaitis et al., N. Engl. J. Med., 324(25):1773-1778, 1991.
Nicolas and Rubenstein, In: Vectors: A survey of molecular cloning vectors and their uses, Rodriguez and Denhardt (Eds.), Stoneham: Butterworth, 494-513, 1988.
Nicolau and Sene, Biochim. Biophys. Acta, 721:185-190, 1982.
Nicolau et al, Methods Enzymol., 149:157-176, 1987.
Nishizaki et al. Cancer Res 64:5745-5752, 2004.
Nishizaki et al. Clinical Cancer Research 7: 2887-2897, 2001.
Nishizaki et al. Clinical Cancer Research 5:1015-1023, 1999.
Oliver et al. Cancer Res 63:6643-6650, 2003.
Pan et al., J. Biol. Chem., 281:8600-8606, 2006.
Parker et al., Cancer, 107:37-45, 2006.
Paskind et al., Virology, 67:242-248, 1975.
PCT Appln. WO 00/44914
PCT Appln. WO 01/36646
PCT Appln. WO 01/68836
PCT Appln. WO 0208436
PCT Appln. WO 0231168
PCT Appln. WO 0285287
PCT Appln. WO 0333029
PCT Appln. WO 94/09699
PCT Appln. WO 95/06128
PCT Appln. WO 98/07408
PCT Appln. WO 99/18933
PCT Appln. WO 99/32619
Pelletier and Sonenberg, Nature, 334(6180):320-325, 1988.
Peng, Hum. Gene Ther., 16(9):1016-1027, 2005.
Philip et al., J. Biol. Chem., 268(22):16087-16090, 1993.
Physicians Desk Reference
Pivot et al., Oncology, 61(3):197-204, 2001.
Post et al., Cell, 24(2):555-65, 1981.
Potter et al., Proc. Natl. Acad. Sci. USA, 81:7161-7165, 1984.
Racher et al., Biotechnology Techniques, 9:169-174, 1995.
Ragot et al., Nature, 361:647-650, 1993.
Recondo et al., Laryngoscope, 101(5):494-501, 1991.
Reinhart et al., Nature, 403:901-906, 2000.
Reinhold-Hurek and Shub, Nature, 357:173-176, 1992.
Remington's Pharmaceutical Sciences, 15^{th} ed., pages 1035-1038 and 1570-1580, Mack Publishing Company, Easton, PA, 1980.
Renan, Radiother. Oncol., 19:197-218, 1990.
Rich et al., Hum. Gene Ther., 4:461-476, 1993.
Ridgeway, In: Vectors: A survey of molecular cloning vectors and their uses, Rodriguez and Denhardt (Eds.), Stoneham:Butterworth, 467-492, 1988.
Rippe et al., Mol. Cell Biol., 10:689-695, 1990.
Rosato et al., Int. J. Cancer, 119:1717-1722, 2006.
Rosenfeld et al., Science, 252:431-434, 1991.
Rosenfeld, et al., Cell, 68:143-155, 1992.
Roth et al., Nat Med., 2(9):985-991, 1996.
Roth Expert Opin Biol Ther.6(1):55-61, 2006.
Roux et al., Proc. Natl. Acad. Sci. USA, 86:9079-9083, 1989.
Sambrook et al., In: Molecular cloning, Cold Spring Harbor Laboratory Press, Cold Spring Harbor, NY, 2001.
Sano et al., Pathol. Int., 50(9):709-716, 2000.
Sarkis et al., J. Clin. Oncol., 13(6):1384-1390, 1995.
Sarver et al., Science, 247:1222-1225, 1990.
Sauter et al., J. Surg. Oncol., 58(4):269-273, 1995.
Saxena et al., J. Biol. Chem., 278:44312-44319, 2003.
Scanlon et al., Proc. Natl. Acad. Sci. USA, 88:10591-10595, 1991.
Schaefer et al., Cell Signal, 12(3):143-151, 2000.
Sharp and Zamore, Science, 287:2431-2433, 2000.
Sharp, Genes Dev., 13:139-141, 1999.
Sharma et al., Head Neck.,26:733-740, 2004.
Shaw et al., Proc. Natl. Acad. Sci. USA, 89(10):4495-4499, 1992.
Shieh et al., Cell, 91:325-334, 1997.
Simon, Control Clin. Trials, 10(1): 1-10, 1989.
Smith and Moss, Gene, 25(1):21-28, 1983.
Smith and Rutledge, Natl. Cancer Inst. Monogr., 42:141-143, 1975.
Solodin et al., Biochemistry, 34(41):13537-13544, 1995.
Soo et al., J. Cell. Biochem., 74(1):1-10, 1999.
Stenmark-Askmalm et al., Br. J. Cancer, 72(3):715-719, 1995.
Stratford-Perricaudet and Perricaudet, In: Human Gene Transfer, Eds, Cohen-Haguenauer and Boiron, John Libbey Eurotext, France, 51-61, 1991.
Stratford-Perricaudet et al., Hum. Gene. Ther., 1:241-256, 1990.
Swisher et al., J. Natl. Cancer Inst., 91(9):763-771, 1999.
Swisher et al., Clin Cancer Res 9:93-101, 2003.
Tabara et al., Cell, 99(2):123-132, 1999.
Tango et al., Cancer Sci., 95(5):459-463, 2004.
Temin, In: Gene Transfer, Kucherlapati (Ed.), NY, Plenum Press, 149-188, 1986.
Templeton et al., Nat. Biotechnol., 15(7):647-652, 1997.
Thierry et al., Proc. Natl. Acad. Sci. USA, 92(21):9742-9746, 1995.
Tibbetts et al., Genes Dev., 13:152-157, 1999.
Top etal., J. Infect. Dis., 124:155-160, 1971.
Tratschin et al., Mol. Cell. Biol., 4:2072-2081, 1984.
Tsujimoto and Croce, Proc. Natl. Acad. Sci. USA, 83(14):5214-5218, 1986.
Tsujimoto et al., Science, 228(4706):1440-1443, 1985.
Tsukamoto et al., Nat. Genet., 9(3):243-248, 1995.
Tur-Kaspa et al., Mol. Cell Biol., 6:716-718, 1986.
Weinberg, Science, 254(5035):1138-1146, 1991.
Wilcock and Lane, Nature, 349(6308):429-431, 1991.
Wilder et al., Cancer Res., 59:410-413, 1999a.
Wilder et al., Gene Therapy, 6:57-62, 1999b.
Wilson et al., Science, 244:1344-1346, 1989.
Wincott et al., Nucleic Acids Res., 23(14):2677-2684, 1995.
Wong et al., Gastroenterology, 130(1):73-79, 2006)
Wong et al., Gene, 10:87-94, 1980.
Wu and Wu, Biochemistry, 27:887-892, 1988.
Wu and Wu, J. Biol. Chem., 262:4429-4432, 1987.
Xia et al., Cancer Res., 66:3392-3395, 2006.
Xiong et al. Nature 366:701-704, 1993.
Xu et al., Curr. Biol., 13:790-795, 2003.
Yang and Huang, Gene Therapy, 4 (9):950-960, 1997.
Yonish-Rouach et al., Nature, 352(6333):345-347, 1991.
Young et al., N Engl J Med. 7:299(23):1261-1266, 1978.
Zakut-Houri et al., EMBO J., 4(5):1251-1255, 1985.
Zeng et al., Mol. Cell, 9:1327-1333, 2002.
Zeng et al., Proc. Natl. Acad. Sci. USA, 100:9779-9784, 2003.
Zhang et al., Endocrinology, 141:4698-4710, 2000.
Zhang et al., Hum. Gene Ther., 6(2):155-164, 1995.
Zhao et al., J Zhejiang Univ SCIENCE B 6(12):1163-1169, 2005.
Zhu et al., Science, 261(5118):209-211, 1993.
Zufferey et al., Nat. Biotechnol., 15(9):871-875, 1997.

1. A method of providing a clinical benefit to a subject suffering from a tumor comprising:
(a) assessing a gene therapy treatment outcome indicator in the subject, wherein the presence of the gene therapy treatment outcome indicator correlates with clinical benefit following gene therapy;
(b) making a treatment decision based on step (a); and
(c) treating the subject with said gene therapy if the subject exhibits the gene therapy outcome indicator.

2. The method of claim 1, wherein the gene therapy is a p53 therapy.

3. The method of claim 2, wherein the p53 therapy is Advexin.

4. The method of claim 1, wherein assessing the gene therapy treatment outcome indicator comprises detection of p53 protein expression in a tumor cell from said subject, wherein detectable p53 prior to gene therapy correlates with clinical benefit following gene therapy.

5. The method of claim 1, wherein assessing the gene therapy treatment outcome indicator comprises detecting p14ARF and/or hdm-2 expression in a tumor cell from said subject, wherein a normal or higher expression of p14ARF and/or normal or lower expression of hdm-2 prior to gene therapy, as compared to a control cell, correlate with clinical benefit following gene therapy.

6. The method of claim 1, wherein the gene therapy treatment outcome indicator is one or more of the following factors for the subject:
(i) interval from end of first treatment after diagnosis to relapse (PFI);
(ii) tumor diameter;
(iii) tumor-associated pain;
(iv) tumor necrosis of target lesions;
(v) localization of the primary tumor;
(vi) prior chemotherapy or radiotherapy;
(vii) Karnofsky performance scale (KPS);
(viii) weight loss;
(ix) low serum albumin level; and/or (x) assessing target lesions in a prior irradiated field,
wherein a PFI of greater than about 12 months, a tumor diameter of less than about 50 mm, minimal or absence of pain, absence of tumor necrosis of target lesions, absence of non-localized disease, prior exposure to chemotherapy or radiotherapy, a KPS of greater than about 90%, minimal or no prior weight loss, normal or near normal serum albumin, and the presence of target lesions in a prior irradiated field, predict clinical benefit following gene therapy.

7. The method of claim 1, wherein the tumor is a benign tumor growth.

8. The method of claim 7, wherein the benign tumor growth is benign prostatic hyperplasia, oral leukoplakia; a colon polyp, an esophageal pre-cancerous growthor a benign lesion.

9. The method of claim 1, wherein the tumor is cancer.

10. The method of claim 9, wherein the cancer is an oral cancer, oropharyngeal cancer, nasopharyngeal cancer, respiratory cancer, a urogenital cancer, a gastrointestinal cancer, a central or peripheral nervous system tissue cancer, an endocrine or neuroendocrine cancer or a hematopoietic cancer.

11. The method of claim 9, wherein the cancer is a glioma, a sarcoma, a carcinoma, a lymphoma, a melanoma, a fibroma, or a meningioma.

12. The method of claim 9, wherein the cancer is brain cancer, oropharyngeal cancer, nasopharyngeal cancer, renal cancer, biliary cancer, prostatic cancer, pheochromocytoma, pancreatic islet cell cancer, Li-Fraumeni tumors, thyroid cancer, parathyroid cancer, pituitary tumors, adrenal gland tumors, osteogenic sarcoma tumors, multiple neuroendrcine type I and type II tumors, breast cancer, lung cancer, head & neck cancer, prostate cancer, esophageal cancer, tracheal cancer, skin cancer brain cancer, liver cancer, bladder cancer, stomach cancer, pancreatic cancer, ovarian cancer, uterine cancer, cervical cancer, testicular cancer, colon cancer, rectal cancer or skin cancer.

13. The method of claim 1, wherein clinical benefit comprises reduction in tumor size or burden, blocking of tumor growth, reduction in tumor-associated pain, long-term non-progression, induction of remission, reduction of metastasis, or increased patient survival.

14. The method of claim 1, wherein the gene therapy is a tumor suppressor gene therapy, a cell death protein gene therapy, a cell cycle regulator gene therapy, a cytokine gene therapy, a toxin gene therapy, an immunogene therapy, a suicide gene therapy, a prodrug gene therapy, an anti-cellular proliferation gene therapy, an enzyme gene therapy, or an anti-angiogenic factor gene therapy.

15. The method of claim 14, wherein the tumor suppressor therapy is APC, CYLD, HIN-1, KRAS2b, p16, p19, p21, p27, p27mt, p53, p57, p73, PTEN, FHIT, Rb, Uteroglobin, Skp2, BRCA-1, BRCA-2, CHK2, CDKN2A, DCC, DPC4, MADR2/JV18, MEN1, MEN2, MTS1, NF1, NF2, VHL, WRN, WT1, CFTR, C-CAM, CTS-1, zac1, ras, MMAC1, FCC, MCC, FUS1, Gene 26 (CACNA2D2), PL6, Beta* (BLU), Luca-1 (HYAL1), Luca-2 (HYAL2), 123F2 (RASSF1), 101F6, or Gene 21 (NPRL2).

16. The method of claim 14, wherein the pro-apoptotic protein therapy is mda7, CD95, caspase-3, Bax, Bag-1, CRADD, TSSC3, bax, hid, Bak, MKP-7, PARP, bad, bcl-2, MST1, bbc3, Sax, BIK, or BID.

17. The method of claim 14, wherein the cell cycle regulator therapy is an antisense oncogene, an oncogene siRNA, an oncogene single-chain antibody, or an oncogene ribozyme.

18. The method of claim 14, wherein the cytokine therapy is GM-CSF, G-CSF, IL-1α, IL-1β, IL-2, IL-3, IL-4, IL-5, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32 IFN-α, IFN-β, IFN-γ, MIP-1α, MIP-1β, TGF-β, TNF-α, TNF-β, or PDGF.

19. The method of claim 14, wherein the anti-angiogenic therapy is angiostain, endostain, avastin or an antisense, siRNA, single-chain antibody, or a ribozyme against a pro-angiogenic factor.

20. The method of claim 1, wherein the cancer has normal p53 protein or gene strucutre, or p53 protein function.

21. The method of claim 1, wherein the cancer has abnormal p53 protein or gene strucutre, or p53 protein function.

22. The method of claim 14, wherein the gene therapy is delivered by a non-viral vector.

23. The method of claim 22, wherein the non-viral vector is entrapped in a lipid vehicle.

24. The method of claim 23, wherein the lipid vehicle is a liposome.

25. The method of claim 22, wherein the vehicle is a nanoparticle.

26. The method of claim 1, where the gene therapy is delivered by a viral vector.

27. The method of claim 26, wherein the viral vector is a retroviral vector, an adenoviral vector, an adeno-associated viral vector, a pox viral vector, a polyoma viral vector, a lentiviral vector, or a herpesviral vector.

28. The method of claim 6, wherein the subject exhibits a higher expression of p53.

29. The method of claim 28, wherein the subject further exhibits 2 or more of the factors (i)-(x) that correlate with clinical benefit.

30. The method of claim 28, wherein the subject further exhibits 4 or more of the factors (i)-(x) that correlate with clinical benefit.

31. The method of claim 28, wherein the subject further exhibits 6 or more of the factors (i)-(x) that correlate with clinical benefit.

32. The method of claim 28, wherein the subject further exhibits 8 of the factors (i)-(x) that correlate with clinical benefit.

33. The method of claim 28, wherein the subject further exhibits 10 of the factors (i)-(x) that correlate with clinical benefit.

34. The method of claim 28, wherein an additional factor is (i).

35. The method of claim 28, wherein an additional factor is (ii).

36. The method of claim 1, wherein p53 expression is assessed.

37. The method of claim 1, wherein said gene therapy is loco-regional gene therapy.

38. The method of claim 37, wherein the loco-regional gene therapy comprises localized gene therapy.

39. The method of claim 38, wherein the localized gene therapy comprises direct injection of the tumor.

40. The method of claim 38, wherein the localized gene therapy comprises injection of tumor vasculature.

41. The method of claim 37, wherein the loco-regional gene therapy comprises regional gene therapy.

42. The method of claim 41, wherein the regional gene therapy comprises administration into a tumor-associated lymph vessel or duct.

43. The method of claim 37, wherein the administration comprises intraperitoneal, intrapleural, intravesicular, or intrathecal administration.

44. The method of claim 41, wherein the regional gene therapy comprises administration into the vasculature system of a limb associated with the tumor.

45. The method of claim 1, wherein the assessing comprises immunohistochemistry of a tumor sample.

46. The method of claim 1, wherein the assessing comprises an ELISA, an immunoassay, a radioimmunoassay (RIA), an immunoradiometric assay, a fluoroimmunoassay, an immunoassay, a chemiluminescent assay, a bioluminescent assay, a gel electrophoresis, a Western blot analysis or an in situ hybridization assay of a tumor sample.

47. The method of claim 1, wherein the assessing comprises antibody detection of p53 in a tumor cell lysate.

48. The method of claim 1, wherein the assessing comprises amplification of a p53 transcript.

49. The method of claim 1, wherein assessing comprises antibody detection of p14ARF and/or hdm-2 in a tumor cell lysate.

50. The method of claim 1, wherein assessing comprises amplification of a p14ARF and/or an hdm-2 transcript.

51. The method of claim 48, wherein amplification comprises RT-PCR.

52. The method of claim 1, wherein assessing comprises in situ hybridization, Northern blotting or nuclease protection.

53.-71. (canceled)
